(19) United States
(12) Patent Application Publication
(10) Pub. No.: US 2021/0032369 A1
(43) Pub. Date: Feb. 4, 2021

(54) METHODS RELATED TO PARKINSON?S DISEASE AND SYNUCLEINOPATHIES

(71) Applicant: The Scripps Research Institute, La Jolla,, CA (US)

(72) Inventors: Corinne Lasmezas, Palm Beach Gardens, FL (US); Minghai Zhou, Jupiter, FL (US); Diego Grassi, Bordeaux (FR)

(21) Appl. No.: 16/969,020

(22) PCT Filed: Feb. 12, 2019
(86) PCT No.: PCT/US2019/017718

§ 371 Date: Aug. 11, 2020

Related U.S. Application Data
(60) Provisional application No. 62/629,503, filed on Feb. 12, 2018.

(51) Int. Cl. C07K 16/44; G01N 33/50; G01N 33/68

(52) U.S. Cl. CPC C07K 16/44; G01N 33/502; G01N 2800/2835; G01N 33/6896; C07K 2317/34; G01N 33/5088

(57) ABSTRACT

The invention provides methods for generating and screening agents that are useful for treating, diagnosing and monitoring Parkinson's Disease and other synucleinopathies.

### CROSS REFERENCE TO RELATED APPLICATIONS

This application claims the benefit of priority under 35 U.S.C. § 119(e) to U.S. Provisional Application No. 62/529,503, filed on Feb. 12, 2018, which is incorporated herein by reference in its entirety.

### STATEMENT OF GOVERNMENT SUPPORT

This invention was made with government support under Contract number 5R01NS085223 awarded by the National Institutes of Health. The government has certain rights in the invention.

### BACKGROUND

Parkinson's disease (PD) is an age-related protein misfolding neurodegenerative disease (PMND) affecting over 1 million people in the United States alone. It is the second most common neurodegenerative disorder after Alzheimer's disease (AD). Approximately 1-2% of the population over the age of 60 and 4-5% over the age of 85 suffers from PD. PD is characterized by resting tremor, bradykinesia, rigidity, gait disturbance and postural instability. Motor impairment is due to the degeneration of dopaminergic neurons of the substantia nigra pars compacta (SNpc) and subsequent loss of dopamine innervation in the striatum. Affected neurons accumulate intracytoplasmic inclusions known as Lewy bodies (LBs).

Approximately 85-90% of PD cases are considered idiopathic. At least five genes, SNCA, GBA, PARK2/Parkin, PINK1, DJ-1, LRRK2, are linked to heritable PD. Alpha-synuclein (α-syn), encoded by the SNCA gene in PD, is the major component of LBs and plays a central role in the pathogenesis of PD. The intracellular accumulation of α-synuclein is also the hallmark of several disorders referred to as synucleinopathies, such as dementia with Lewy bodies, the Lewy body variant of AD, or multiple-system atrophy.

There is currently no disease-modifying treatment for PD and synucleinopathies. The instant invention is directed to addressing this and other unmet needs in the art.

### SUMMARY

In one aspect, the invention provides methods of generating antibodies useful for treating Parkinson's disease (PD) and other synucleinopathies. The methods involve (a) immunizing a non-human animal with an immunogen composition comprising an alpha-synuclein (α-syn) derived polypeptide or a polymer exhibiting the same conformational epitope as the polypeptide, and (b) isolating one or more antibodies that specifically recognize the polypeptide. In these methods, the α-syn derived polypeptide comprises a conformationally distinct and nonfibrillar α-syn variant with mitotoxicity. In some embodiments, the α-syn derived polypeptide contains phosphorylated Ser¹²⁹. In some embodiments, the employed α-syn derived polypeptide is immunoreactive with anti-phospho-Ser129 antibody GTX50222, lot 821505177. Additionally or alternatively, the α-syn derived polypeptide employed in these embodiments is not immunoreactive with fibrillar Pα-synF-recognizing 81A and/or antibody MJF-R13.

In some methods, the employed α-syn derived polypeptide is an α-syn variant with a deletion of about 0 to 25 N-terminal amino acid residues and/or a deletion of about 0 to 25 C-terminal amino acid residues relative to a full length α-syn protein. In some embodiments, the employed α-syn derived polypeptide has mitotoxicity in inducing mitochondrial dysfunction and structural damage resulting in mitophagy. In some embodiments, the α-syn variant induces the formation of small aggregates of phosphorylated Acetyl-CoA carboxylase (ACC) and induces the phosphorylation of glycogen synthase kinase 3 beta (GSK3β) and mitogen-activated protein kinases (MAPKs) such as mitogen-activated protein kinase kinase 4 (MKK4), c-Jun N-terminal kinase (JNK), p38 and extracellular signal-regulated kinase 5 (ERK5). In some embodiments, the α-syn variant induces the formation of small aggregates of phosphorylated tau (ptau). In some embodiments, the α-syn variant induces synaptic damage and loss of dendritic spines. In certain embodiments, the α-syn variant (termed herein Pα-syn*) triggers the activation of several MAPKs including MKK4, JNK, pERK5 and p38 as well as the phosphorylation of tau at the mitochondrial membrane. In various embodiments, the α-syn derived polypeptide can be extracted from Pα-syn* inclusions present in a cell culture, brains of animal models of PD and other synucleinopathies, or brains of patients with PD and other synucleinopathies. In some embodiments, the immunogen composition further contains an adjuvant. In some methods, the antibodies are isolated by phage display. In some methods, the isolated antibodies are further examined for a therapeutic activity. For example, the antibodies can be examined for inhibition of a toxic activity in a cellular model of synucleinopathy or reduction in the generation and propagation of pathogenic phosphorylated α-syn. In some methods, the polypeptide immunogen is derived from a human α-syn, e.g., human α-syn as shown in SEQ ID NO:1. In some embodiments, the polypeptide immunogen is encoded by the nucleic acid sequence of SEQ ID NO: 1 or a variant thereof having at least about 50% (such as at least about any of 55%, 60%, 65%, 70%, 75%, 80%, 85%, 86%, 87%, 88%, 89%, 90%, 91%, 92%, 93%, 94%, 95%, 96%, 97%, 98%, 99%, or greater) sequence identity to SEQ ID NO: 1. In certain embodiments, the human α-syn comprises at least a 50% sequence identity to SEQ ID NO:1, variants or fragments thereof. In certain embodiments, the human α-syn comprises SEQ ID NO:1, variants or fragments thereof.

In another aspect, the invention provides methods for identifying potential therapeutic agents for treating PD and other synucleinopathies. These methods involve (a) contacting with or administering to a cell or animal model of PD and other synucleinopathies a plurality of candidate agents, (b) detecting in a specific candidate agent-treated model a disruption or decreased formation of an alpha-synuclein (α-syn) derived polypeptide relative to untreated control model. Alternatively, (b) can involve detecting binding of a candidate agent to an alpha-synuclein (α-syn) derived polypeptide specific to the candidate agent-treated relative to untreated control model. In these methods, the α-syn derived polypeptide comprises a conformationally distinct and nonfibrillar α-syn variant with mitotoxicity. In some of these methods, the employed α-syn derived polypeptide contains phosphorylated Ser¹²⁹. In some of these methods, the α-syn derived polypeptide is an α-syn variant with a deletion of about 0 to 25 N-terminal amino acid residues and/or a deletion of about 0 to 25 C-terminal amino acid residues relative to a full length α-syn protein. In some embodiments, the mitotoxicity of the employed α-syn variant is inducing mitochondrial dysfunction and structural damage resulting in mitophagy. In some embodiments, the α-syn variant induces the formation of small aggregates of phosphorylated Acetyl-CoA carboxylase (ACC) and induces the phosphorylation of glycogen synthase kinase 3 beta (GSK3β) and mitogen-activated protein kinases (MAPKs) such as mitogen-activated protein kinase kinase 4 (MKK4), c-Jun N-terminal kinase (JNK), p38 and extracellular signal-regulated kinase 5 (ERK5). In some embodiments, the α-syn variant induces the formation of small aggregates of phosphorylated tau (ptau). In some embodiments, the α-syn variant induces synaptic damage and loss of dendritic spines. In certain embodiments, the α-syn variant (termed herein Pα-syn*) triggers the activation of several MAPKs including MKK4, JNK, pERK5 and p38 as well as the phosphorylation of tau at the mitochondrial membrane. In some methods, the employed α-syn variant polypeptide is derived from a human α-syn, e.g., human α-syn as shown in SEQ ID NO:1. In some embodiments, the polypeptide immunogen is encoded by the nucleic acid sequence of SEQ ID NO: 1 or a variant thereof having at least about 50% (such as at least about any of 55%, 60%, 65%, 70%, 75%, 80%, 85%, 86%, 87%, 88%, 89%, 90%, 91%, 92%, 93%, 94%, 95%, 96%, 97%, 98%, 99%, or greater) sequence identity to SEQ ID NO: 1.

In another aspect, the invention provides methods of diagnosing or monitoring disease progression in patients affected by PD and other synucleinopathies. These methods entail detecting in the patients the presence and/or quantifying the amount of a conformationally distinct and nonfibrillar α-syn variant with mitotoxicity. In some embodiments, the α-syn variant induces the formation of small aggregates of phosphorylated Acetyl-CoA carboxylase (ACC) and induces the phosphorylation of glycogen synthase kinase 3 beta (GSK3β) and mitogen-activated protein kinases (MAPKs) such as mitogen-activated protein kinase kinase 4 (MKK4), c-Jun N-terminal kinase (JNK), p38 and extracellular signal-regulated kinase 5 (ERK5). In some embodiments, the α-syn variant induces the formation of small aggregates of phosphorylated tau (ptau). In some embodiments, the α-syn variant induces synaptic damage and loss of dendritic spines. In certain embodiments, the α-syn variant (termed herein Pα-syn*) triggers the activation of several MAPKs including MKK4, JNK, pERK5 and p38 as well as the phosphorylation of tau at the mitochondrial membrane. In some embodiments, the employed α-syn variant contains phosphorylated Ser¹²⁹. In some embodiments, the employed α-syn variant contains a deletion of about 0 to 25 N-terminal amino acid residues and/or a deletion of about 0 to 25 C-terminal amino acid residues relative to a full length α-syn protein. In some embodiments, the mitotoxicity of the employed α-syn variant is inducing mitochondrial dysfunction and structural damage resulting in mitophagy. In some embodiments, the diagnosis or disease monitoring is performed with a tissue or body fluid sample obtained from subjects affected by PD and other synucleinopathies.

In still another aspect, the invention provides engineered cells or transgenic non-human animals that contain a transgene encoding an alpha-synuclein (α-syn) derived polypeptide. The α-syn derived polypeptide contains of a deletion of about 0 to 25 N-terminal amino acid residues and a deletion of about 0 to 25 C-terminal amino acid residues of a full length α-syn protein. In some embodiments, the engineered cell is a neuronal cell. In some embodiments, the transgenic non-human animal is a rodent.

In another aspect, the invention provides methods for generating small molecules useful for treating Parkinson's disease (PD) and other synucleinopathies. These methods entail (a) performing structure-based drug design directed towards the conformational epitope of an immunogen composition comprising an alpha-synuclein (α-syn) derived polypeptide or a polymer exhibiting the same conformational epitope as the polypeptide, and (b) selecting a small molecule specifically recognizing the conformational epitope of an α-syn derived polypeptide. In these methods, the α-syn derived polypeptide contains a conformationally distinct and nonfibrillar α-syn variant with mitotoxicity. In some embodiments, the α-syn derived polypeptide contains phosphorylated Ser¹²⁹. In some embodiments, the α-syn derived polypeptide is immunoreactive with anti-phospho-Ser129 antibody GTX50222, lot 821505177. Additionally or alternatively, the employed the α-syn derived polypeptide is not immunoreactive with fibrillar Pα-synF-recognizing 81A and/or antibody MJF-R13. In some embodiments, the employed α-syn derived polypeptide is an α-syn variant with a deletion of about 0 to 25 N-terminal amino acid residues and/or a deletion of about 0 to 25 C-terminal amino acid residues relative to a full length α-syn protein. In some methods, mitotoxicity of the α-syn derived polypeptide is inducing mitochondrial dysfunction and structural damage resulting in mitophagy. In some embodiments, the α-syn variant induces the formation of small aggregates of phosphorylated Acetyl-CoA carboxylase (ACC) and induces the phosphorylation of glycogen synthase kinase 3 beta (GSK3β) and mitogen-activated protein kinases (MAPKs) such as mitogen-activated protein kinase kinase 4 (MKK4), c-Jun N-terminal kinase (JNK), p38 and extracellular signal-regulated kinase 5 (ERK5). In some embodiments, the α-syn variant induces the formation of small aggregates of phosphorylated tau (ptau). In some embodiments, the α-syn variant induces synaptic damage and loss of dendritic spines. In certain embodiments, the α-syn variant (termed herein Pα-syn*) triggers the activation of several MAPKs including MKK4, JNK, pERK5 and p38 as well as the phosphorylation of tau at the mitochondrial membrane. In various embodiments, the α-syn derived polypeptide can be extracted from Pα-syn* inclusions present in a cell culture, brains of animal models of PD and other synucleinopathies, or brains of patients with PD and other synucleinopathies. In some embodiments, the methods further include examining the selected small molecule for a therapeutic activity, e.g., inhibition of a toxic activity in a cellular model of synucleinopathy or a reduction in the generation and propagation of pathogenic phosphorylated α-syn. In some embodiments, the employed α-syn variant is derived from a human α-syn, e.g., human α-syn as shown in SEQ ID NO:1. In some embodiments, the polypeptide immunogen is encoded by the nucleic acid sequence of SEQ ID NO: 1 or a variant thereof having at least about 50% (such as at least about any of 55%, 60%, 65%, 70%, 75%, 80%, 85%, 86%, 87%, 88%, 89%, 90%, 91%, 92%, 93%, 94%, 95%, 96%, 97%, 98%, 99%, or greater) sequence identity to SEQ ID NO: 1.

A further understanding of the nature and advantages of the present invention may be realized by reference to the remaining portions of the specification and claims.

### BRIEF DESCRIPTION OF THE DRAWINGS

FIG. 1: Time-course of appearance of a non-fibrillar phosphorylated α-syn conformer (Pα-syn*) in PFF-treated neurons. Primary hippocampal mouse neurons were exposed to preformed fibrils (PFFs) at DIV7 and examined by ICC at various time points from day 2 to 14. Cells similarly treated with PBS alone constitute the control. Pictures show labeling with the Pα-syn antibodies recognizing Pα-syn*or Pα-synF, respectively, and DAPI staining showing the nuclei, color-coded as green, red and blue, respectively, in the merged image. Neurons from the PBS control were labeled similarly, the merged image is shown. No Pα-syn was observed in control cells in our experimental conditions. Scale bars=10 μm.

FIG. 2: Detection of both Pα-synF and Pα-syn* in the brains of PFF-injected mice and PD patients. Upper panels: Mouse hippocampal or cortical primary neurons seeded with PFFs both develop Pα-synF and Pα-syn* inclusions. Middle panels: Mice stereotaxically injected with PFFs in the striatum develop both Pα-synF and Pα-syn* inclusions in the cortex and substantia nigra, with morphologies and subcellular localization identical to the cell cultures. Lower panels: Both Pα-synF and Pα-syn* inclusions were observed in the cortex of 3 LB harboring patients with PD (Table 1, top panels: case ID10-89; middle panel: case ID12-69, bottom panel: case ID11-51). Pα-syn* was also observed in all but one “high LB” cases and in two “low LB” that were looked at. Note the Pα-syn* puncta surrounding the LBs. The LBs are detected using the Pα-synF specific antibody. Labeling for Pα-synF, Pα-syn* and DAPI was color-coded as green, red and blue, respectively, in the merged image. Scale bars=20 μm.

FIG. 3: Pα-syn* originates from partial degradation of Pα-synF fibrils. (A-D) Thin Pα-synF fibrils undergo conversion into Pα-syn* in a patchy manner throughout the fibrillar core leading to the progressive disappearance of the Pα-synF core and release of granular and serpentine Pα-syn* structures. (C) Pα-syn* “beads” (arrow). (D) Pα-synF core has entirely disappeared, leaving behind ribbons of Pα-syn*. (E-G) Degradation of thick intertwined Pα-synF fibrils: one strand is degraded first, leading to the release of granular Pα-syn* surrounding the remaining strand. (H) A thick and straight Pα-synF fibril is converted into Pα-syn* starting from one end. The middle panel of the high magnification images shows the degradation front in the fibrillar core. Cells were labeled with Pα-synF, Pα-syn* or p62 antibodies, color-coded as green, red and blue in the merged image. Note that p62 labeling is strictly restricted to Pα-synF showing that Pα-synF undergoes autophagy while Pα-syn* seems to be the product of the autophagic process. (I) The western blots show detection of total α-syn using antibodies directed towards the N-terminus, the central region and the C-terminus of α-syn recognizing the epitopes indicated in the left scheme, as well as Pα-syn* using the Pα-syn* specific antibody (epitope indicated in the right scheme). Pα-syn*, labelled by red arrows, migrates at 12.5 kDa and is present specifically in PFF-treated neurons. A Pα-syn* dimer is also detected at 25 kDa and is also specific for PFF-treated neurons. Pα-syn* is present in both the soluble (TX-100 extracted) and insoluble (SDS extracted) cellular fractions. A faint band corresponding to ubiquitinated Pα-syn* is detectable at 13 kDa with the Pα-syn* antibody, likely corresponding to immature Pα-syn* attached to Pα-synF, since mature Pα-syn* aggregates are not ubiquitinated. Pα-syn* is also detectable using the N-terminal, central domain and C-terminal total α-syn antibodies used for mapping purposes. Pα-synF is detected by a specific Pα-synF antibody (epitope indicated in the right scheme) at 15 kDa corresponding to the full-length protein, as previously described, as well as at 17 kDa corresponding to the ubiquitinated form (green arrows). Scale bars=5 μm.

FIG. 4: Association of Pα-synF and Pα-syn* with markers of the autophagolysosomal pathway indicates that Pα-syn* is the autophagic product of Pα-synF. (A) Pα-synF fibrils are entirely covered with ubiquitin, tagging them for degradation (see also colocalization analysis in FIG. 16). (B) Pα-synF fibrils are tagged with the adaptor protein p62, targeting them for autophagic degradation. Arrows show nascent Pα-syn* inclusions in direct contact with p62 tagged Pα-synF fibrils. (C) LC3 covers Pα-synF fibrils. Arrows show nascent Pα-syn* inclusions in direct contact with LC3 tagged Pα-synF fibrils. (D) Pα-syn* inclusions are contained in LAMP1 positive vesicles. Cells were labeled with Ubiquitin, p62, LC3, LAMP1 and Pα-syn*antibodies and DAPI, color-coded as green, red and blue in the merged image. Scale bars=10 μm.

FIG. 5: Pα-syn* is found in autophagolysosomes and lysosomes. (A-C) Pα-synF aggregates are engulfed in LAMP1 vesicles (autophagolysosomes or lysosomes) at days 2-3, before fibrils are seen in the cells; small Pα-syn* puncta are seen in B and C. (D-F) Short protofibrils of Pα-synF are being engulfed by LAMP1 vesicles. (G) Situation where a thin Pα-synF fiber is being degraded within its core as shown in FIG. 3 (A-D), leading to an overlap of ubiquitin and LAMP1 staining (arrows). Pα-syn* detached from the core is not ubiquitin labeled (arrowhead) (H) A thick Pα-synF fiber is releasing Pα-syn*; there is no overlap between ubiquitin staining of the fibrillar core and Pα-syn* labeling. The arrowhead shows an indent in the fibril coinciding with the presence of a lysosome. (I-K) Pα-syn* is seen exiting lysosomes (arrows point to Pα-syn* aggregates exiting disrupted lysosomes). (L) Elongated vesicles positive for Lysotracker DND-99 and weakly labeled with LAMP1 contain Pα-syn* inclusions, organized in a chain-like shape, are likely to be autophagolysosomes degrading a fibril (FIG. 5L, arrowheads in the left inset). On the other hand, in LAMP1-positive Pα-syn*-laden vesicles (FIG. 5L, arrows in the right inset) Lysotracker DND-99 staining is absent, indicating lack of the acidic internal environment of those organelles. In contrast, most LAMP1-positive, Pα-syn*-negative vesicles in the close vicinity show a strong signal for Lysotracker DND-99. Cells were labeled with Pα-synF and ubiquitin antibodies or Lysotracker DND-99, Pα-syn* and LAMP1 antibodies or DAPI, color-coded as green, red, blue and turquoise in the merged image. Scale bars=5 μm (A-C; D-F; I-K), 10 μm (G; H; L).

FIG. 6: Autophagy modulation alters the production of Pα-syn*. (A-D) Neurons were treated from day 3.5 post-PFF exposure to day 6 post-PFF exposure with the vehicle (A), rapamycin (B), chloroquine (C) or 3-MA (D) as indicated. Note the numerous and large Pα-syn* aggregates in (B). (C) The neuron on the right of the image (right zoom area) shows few Pα-syn* aggregates while the neuron on the left of the image (left zoom area) shows Pα-syn* aggregates in the absence of Pα-synF fibrils. (D) 3-MA treatment leads to fewer Pα-syn* aggregates. (E) The number of Pα-syn* aggregates per cells was counted in 200 cells of each culture in a blinded manner. The graph shows the number of cells bearing few (1-10) to numerous (71-80) aggregates under each culture condition. Cells were labeled with Pα-synF, Pα-syn* and LAMP1 antibodies and DAPI, color-coded as green, red, blue and turquoise in the merged image. Pictures are Z-stacks of 6 confocal images, in order to capture the total number of vesicles present in individual neurons. Scale bars=10 μm.

FIG. 7: Pα-syn* localizes to mitochondria and fragmented mitochondria. (A) Immature serpentine Pα-syn* is present in the vicinity of, but not colocalizing with mitochondria (labelled for Tom20). (B, C) Granular Pα-syn* binds to mitochondrial tubules. (D-G) STED nanoscopic imaging of Pα-syn* aggregates attached to mitochondrial tubules (D, E, F). In (F), arrows point to small Pα-syn* aggregates associated with mitochondrial tubules or circular structures. (G) Large Pα-syn* aggregates are associated with fragmented mitochondria. Cells were labeled with Tom20 antibody and Pα-syn*, color-coded as green and red in the merged image, respectively. Scale bars=5 μm (A-C), 1 μm (D-G).

FIG. 8: Pα-syn* induces loss of mitochondrial membrane potential. (A) Tom20 and Mitotracker CMXRos labeling in PBS-treated cells is overlapping until the ends of the tubules (arrowheads). (B) Pα-syn* labeling colocalizes with Tom20 at the end of the mitochondrial tubule, but Mitotracker CMXRos is disrupted showing a void area (arrow). Cells were labeled with Mitotracker CMXRos, Pα-syn* and Tom20 antibody, color-coded as green, red and blue in the merged images, respectively. Scale bars=1 μm.

FIG. 9: Pα-syn* colocalizes with cytochrome C and pACC1 and is found in areas of mitochondrial-MAM tethering. (A) The points of contact of Pα-syn* with the mitochondria correspond to zones of increased cytochrome C density (arrows in insets). (B) A strong colocalization of Pα-syn* is observed with pACC1 with Pα-syn* inclusions completely overlapping with some pACC1 granules (arrows in insets). (C) Pα-syn* also colocalizes with BiP, indicating that Pα-syn* aggregates are located at the interface between the mitochondrial outer membrane and mitochondria associated ER membranes (MAMs, arrows in insets). (D) Recruitment of pACC1 to the sites of Pα-syn* accumulation at damaged mitochondria. PACC1, Pα-syn* and cytochrome C labeling colocalize, with complete overlap of the Pα-syn* and pACC1 labelling (arrows in insets). (A-C) Cells were labeled with cytochrome C, pACC1, BiP and Pα-syn* antibodies and DAPI, color-coded as green, red and blue in the merged image. (D) Cells were labeled with cytochrome C, Pα-syn*, pACC1 antibodies and DAPI, color-coded as green, red, blue and turquoise in the merged image. (E) Graph showing Manders' correlation coefficients of Pα-syn* aggregates with markers of the outer mitochondrial membrane (Tom20), loss of integrity of mitochondrial membranes (cytochrome C), MAMs (BiP) and pACC1. Manders' coefficient of colocalization was calculated using immunofluorescence intensities recorded in 15 to 20 neurons. Key to statistical significance: ns: non-significant; **: p<0.01; ***: p<0.005; ****: p<0.001. Scale bars=10 μm.

FIG. 10: Pα-syn* triggers mitophagy. (A) LAMP1 positive mitophagic vesicles in an area of mitochondrial network fragmentation contain Pα-syn* and small Tom20 remnants (arrows). Arrowhead shows a large Pα-syn* aggregate colocalizing with Tom20. (B) Parkin, an E3-ubiquitin ligase orchestrating mitophagy at MAMs, colocalizes with Pα-syn* containing LAMP1 positive mitophagic vesicles (arrows). Cells were labeled with Tom20, Parkin, Pα-syn* and LAMP1 antibodies and DAPI, color-coded as green, red, blue and turquoise in the merged image. Scale bars=10 μm. (C) Electron micrograph of Pα-syn* bearing mitophagic vacuoles. Neurons at 14 days post-PFF exposure were examined by IF for Pα-syn* (red) and cytochrome C (green) and by EM for ultrastructural analysis, and images were superimposed. All Pα-syn* aggregates were seen in mitophagic vacuoles also containing small electron dense inclusions probably corresponding to mitochondrial debris. These mitophagic vacuoles were directly abutting cytochrome C positive, fragmented mitochondria. Areas A and B were selected for enlarged images, and two adjacent EM sections are shown (A1-A2 and B1-B2, with and without color overlay). Scale bar=5 μm.

FIG. 11: Life cycle of Pα-syn*. (A) In PFF-seeded neurons, endogenous α-syn misfolds and aggregates in the Pα-synF conformation (depicted in green). (B) Pα-synF forms intertwined fibrils. (C) Pα-synF fibrils undergo autophagic degradation (see FIGS. 3 and 4). However this process is incomplete, generating a Pα-syn species with a different conformation, Pα-syn* (depicted in red). (D) Pα-syn* containing lysosomes are found in the Pα-synF fibrillar core or on the fibril surface (FIGS. 3 & 5). Autophagolysosomes/lysosomes are shown in blue. (E) Pα-syn* aggregates exit the lysosomes (FIG. 5) and localize to mitochondria (FIG. 7). Pα-syn* aggregates colocalize with MAMs, sites of Parkin-dependent mitochondrial fission and mitophagy. They induce mitochondrial membrane depolarization, cytochrome C release, oxidative and energetic stress, formation of pACC aggregates, mitochondrial fragmentation and mitophagy (FIGS. 7-10).

FIG. 12: Colocalization of Pα-syn* with pTau. Neurons at 8 days post PFF/PBS exposure were fixed and labelled for Pαsyn* and for pTau (antibody clone AT8 specific for Tau pS202-T205). Arrows show overlapping of Pαsyn* and pTau signals. Arrowheads show a pTau inclusion juxtaposed with a Pαsyn* inclusion, with a small overlapping area in the middle.

FIG. 13: Alpha-syn monomers do not seed Pα-syn* and Pα-synF aggregation. Primary hippocampal mouse neurons were exposed to α-synuclein monomer at DIV7 and fixed for ICC examination at two time points, day 6 and day 14. Cells similarly treated with PBS alone constitute the control. Pictures show labeling with Pα-syn antibodies recognizing Pα-syn* or Pα-synF, respectively, and DAPI staining showing the nuclei. Neurons corresponding to the PBS control were labeled similarly, but only the merged image is shown. No Pα-syn was observed in either α-synuclein monomer-treated neurons or PBS-treated cells in our experimental conditions. Scale bars=10 μm.

FIG. 14: Pα-synF and Pα-syn* are detected only in neuronal cells. Mouse hippocampal or cortical primary neurons seeded with PFFs both develop Pα-synF and Pα-syn* inclusions. (A-B) Cells were fixed at day 7 after PFFs seeding and labeled with NeuN and Pα-syn* antibodies and DAPI, color-coded as green, red and blue as is shown in the merged image. NeuN is a marker for neuronal cells. Note that Pα-syn* labeling is restricted to NeuN-positive cells, showing that Pα-syn* aggregates are only formed in neurons. (C) Hippocampal neurons were fixed at day 7 after PFFs seeding and labeled with GFAP and Pα-synF antibodies and DAPI, color-coded as green, red and blue in the merged image. GFAP is used as a marker for astrocytes. Note the absence of Pα-synF labeling in astrocytes. Scale bars=50 μm.

FIG. 15: Pα-syn aggregates are found in the cortex and substantia nigra of PFF-injected mouse brains. Adult mice stereotaxically injected with PFFs in the striatum develop both Pα-synF and Pα-syn* inclusions in the cortex and substantia nigra. (A) Mice were euthanized after 30 days of PFFs seeding, the brains were fixed, processed for immunohistochemistry and some brain slices were labeled with Pα-synF and tyrosine hydroxylase (TH) antibodies and DAPI, color-coded as green, red and blue as is shown in the merged image. TH labels dopaminergic neurons of the substantia nigra. Note that Pα-synF labeling is observed mostly in TH-positive cells (arrows). (B) PFF-injected mouse brain slices were processed similar to A and labeled with Pα-synF and Pα-syn* antibodies and DAPI, color-coded as green, red and blue as is shown in the merged image. Note that Pα-synF and Pα-syn* labeling is observed in neurons of the cerebral cortex (arrows). Red pale squares on brain schemes (see merged images) indicate the brain region shown. Scale bars=100 μm.

FIG. 16: Association of the 20S proteasome with Pα-synF and Pα-syn*, and quantitative colocalization studies between Pα-synF/Pα-syn* and markers of proteolytic processing. Mouse hippocampal primary neurons were fixed at day 14 after PFFs seeding and labeled with 20S Proteasome, Pα-syn* and Pα-synF antibodies and DAPI, color-coded as green, red, blue and turquoise as is shown in the merged image. (A) In neurons bearing a high burden of Pα-synF (Pα-synF labelling shown in C), 20S proteasome is rarely found colocalized with α-syn*, and only partially (arrow). (B) In neurons bearing a low burden of Pα-synF (Pα-synF labelling shown in D), colocalization of Pα-syn* with 20S proteasome is frequently observed (arrows). However, low Pα-synF burden cells were less abundant than the high Pα-synF burden cells. (C, D) Pictures are the same as shown in (A) and (B) with the addition of the Pα-synF labelling. In (D), the arrow indicates a neuronal process (probably an axon) belonging to another neuron, different from that bearing Pα-syn* aggregates. Scale bars=10 μm. (E) Graph showing Manders' correlation coefficients of Pα-synF and Pα-syn* aggregates with proteolytic markers. Pα-synF associates tightly with markers of autophagy, while about 80% of Pα-syn* is associated with LAMP1. Both Pα-synF and Pα-syn* can be found associated with the 20S proteasome, showing participation of the proteasome in the degradation of both types Pα-syn aggregates. Note: LC3 antibody yielded week signals, thus the detected Pα-synF staining and the Manders' correlation coefficient for LC3 are considered to be underestimates.

FIG. 17: Pα-syn* is bound to mitochondrial tubules. Z-stack images of Pα-syn* bound to the extremities of mitochondrial tubules. Pα-syn* aggregates seem to be hanging from mitochondrial endings. The disruption of the Mitotracker CMXRos labeling at the mitochondrial endings indicates a local loss of mitochondrial membrane potential in the proximity of Pα-syn*. Scale bar=10 μm.

FIG. 18: Pα-syn* colocalizes with mitochondrial and cellular stress markers at the mitochondria associated ER membranes (MAMs), but not with early endosomes or peroxisomes. (A) Tom20, Pα-syn* and BiP colocalize, illustrating the presence of Pα-syn* aggregates at the points of contact between the mitochondrial outer membrane and MAMs. (B, C) Absence of colocalization with peroxisomes. (B) Pα-syn* colocalization with the mitochondrial outer membrane protein Tom20 shows that Pα-syn* is in direct contact with mitochondria. In contrast, Catalase does not colocalize with Pα-syn*, although some extent of colocalization with Tom20 is observed. The arrow indicates the area of contact between the mitochondrial network and a Pα-syn* tubular structure. (C) Pα-syn* colocalizes with BiP, indicating that Pα-syn* aggregates are located at the interface between the mitochondria associated ER membranes (MAMs, arrow). However, catalase does not colocalize with Pα-syn*. Even in those cells expressing a noticeable amount of catalase and Pα-syn*-positive structures, only areas of juxtaposition between both proteins are observed (arrowhead). (D) Absence of colocalization with early endosomes. Mouse hippocampal primary neurons were seeded with PFFs, fixed at day 7 and labeled with EEA1 and Pα-syn* antibodies and DAPI, color-coded as green, red and blue as is shown in the merged image. Neurons from the PBS control were labeled similarly, but only the merged image is shown. EEA1 was chosen since it is a well-established marker for early endosomes. Despite the presence of numerous EEA1-positive vesicles inside neurons bearing Pα-syn*, there is no colocalization between these 2 proteins.

Mouse hippocampal primary neurons were seeded with PFFs and fixed at day 7. They were labeled with Tom20, BiP/catalase and Pα-syn* antibodies and DAPI, color-coded as green, blue, red and turquoise in the merged image (A, B), with BiP, catalase, Pα-syn* antibodies and DAPI, color-coded as green, blue, red and turquoise in the merged image (C) or with EEA1, Pα-syn* antibodies and DAPI, color-coded as green, red and blue in the merged image (D). Magnification bars=10 μm.

FIG. 19: pJNK colocalizes with Pα-syn* but not Pα-synF. pJNK positive inclusions coincide completely with Pα-syn* inclusions. The intensity of the staining may vary, leading to inclusions exhibiting a predominance of green or red in the merged image. On the other hand, Pα-synF antibody labeled fibrillary structures that excluded pJNK labeling. In insets A1 and B1, several indents in Pα-synF labeling can be seen, corresponding to the formation of Pα-syn* from partial digestion of Pα-synF (Grassi et al., 2018). Pictures show labeling for Pα-syn*, pJNK and Pα-synF in green, red and blue respectively. Scale bars=5 μm.

FIG. 20: P-αsyn* induces MAPK pathway activation. FIG. 20A. pJNK positive inclusions colocalize with MKK4 phosphorylated at T261 (activated MKK4). FIG. 20B. Very few dots corresponding to MKK4 phosphorylated at S80 (inactive MKK4) are detected, however pMKK4 (T80) positive dots colocalize to pJNK positive inclusions. FIGS. 20C-21D. pp38 and pERK5 labelling largely overlaps with pJNK positive inclusions. FIG. 20E. pGSK3β positive dots are detected in close proximity to pJNK positive inclusions with no or only partial overlap. Cells were labeled with phosphorylation-site specific pMKK4, pGSK3β, pp38 or pERK5, and pJNK antibodies and DAPI, color-coded respectively as green, red and blue in the merged image. Scale bars=10 μm.

FIG. 21: P-αsyn* aggregates co-localize with ptau aggregates. FIG. 21A. pJNK localizes to Pα-syn* inclusions. FIGS. 22B-22C. pTau positive inclusions were juxtaposed with or colocalized with pJNK positive inclusions. This was found with both ptau antibodies used, targeting either pS199 (FIG. 21B) or pS202/T205 (FIG. 21C). FIG. 21D. Triple labeling showing the colocalization of Pα-syn* and pJNK, with ptau inclusions being either colocalized or directly juxtaposed. FIGS. 21A-21D. Cells were labeled with phosphorylation-site specific ptau or Pα-syn*, and pJNK antibodies as well as DAPI staining, color-coded respectively as green, red and blue in the merged images. Scale bars=10 μm.

FIG. 22: pJNK colocalizes with Pα-syn* at the mitochondrial membrane. A-B. pJNK positive inclusions, but not Pα-synF fibers, colocalize with Tom20, indicating their association with mitochondrial membranes. C-E. pJNK positive Pα-syn* aggregates colocalize with Tom20. Pictures show cells containing abundant Pα-syn* aggregates, associated with a fragmented mitochondrial network. Pictures show labeling for Tom20, pJNK, Pα-synF or Pα-syn*, and nuclear DAPI staining in green, red and blue respectively. Scale bars=5 μm.

FIG. 23: pTau colocalizes with pJNK positive Pα-syn* aggregates in areas of mitochondrial damage. FIG. 23A. Mitotracker CMXRos staining is absent at the sites of mitochondrial attachment of pJNK positive inclusions. Arrows indicate Tom20 positive areas with interrupted Mitotracker CMXRos staining. FIG. 23B. pJNK positive inclusions colocalize with pACC1 and cytochrome C at the mitochondrial membrane. FIG. 23C. pJNK positive inclusions colocalize with BiP and Tom20, indicating their localization to mitochondria associated ER membranes (MAMs, arrows). FIG. 23D. Colocalization of pJNK positive inclusions and ptau occurs at areas of cytochrome C accumulation indicating damaged mitochondria (arrows). Pictures show labeling for Mitotracker CMXRos/pACC1/BiP/ptau, pJNK, Tom20/cytochrome C and nuclear DAPI staining in green, red, blue and turquoise, respectively. Scale bars=10 μm.

FIG. 24: pJNK positive Pα-syn* aggregates and ptau co-localize in mitophagic vacuoles. FIG. 24A. LAMP1 positive vesicles contain pJNK and Tom20 staining showing that they are mitophagic vacuoles. FIG. 24B. Parkin labeling is associated with pJNK positive inclusions in LAMP1 positive vesicles. FIG. 24C. pJNK positive Pα-syn* aggregates colocalize with ptau and with parkin. Pictures show labeling for Tom20 or parkin, pJNK, LAMP1 or ptau and nuclear DAPI staining in green, red, blue and turquoise, respectively. Scale bars=10 μm.

FIG. 25: Quantitative colocalization studies. FIG. 25A. Graphs showing Manders' correlation coefficients of pJNK with Pα-syn*, ptau and other kinases. pJNK labeling tightly associates with Pα-syn* labeling, but also pMKK4 (activated), pERK5 and pP38 (≥80% colocalization). FIG. 25B. Graph showing Manders' correlation coefficient of Pα-syn* with ptau. FIG. 25C. Graphs showing Manders' correlation coefficients of Pα-syn*, pJNK and ptau with LAMP1 and parkin. FIGS. 25A-25C: Key to statistical analyses: **p<0.01; ***p<0.001; ****p<0.0001. In A, **** indicates p<0.0001 for pGSK3β colocalizing with pJNK significantly less than pMKK4, pp38 and pERK5 colocalize with pJNK. In FIG. 25C, p was 0.05 for the difference between ptau colocalization with LAMP1 and Pα-syn*/pJNK colocalization with LAMP1.

FIG. 26: Model for the molecular cascade and Tau recruitment induced by Pα-syn*, leading to mitochondrial damage and mitophagy. FIG. 26A. Pα-syn* aggregates associate to the mitochondrial membrane, triggering MAPK activation. Pα-syn* binds tau that is phosphorylated by these MAPKs. FIG. 26B. Both Pα-syn* and ptau aggregates grow in size and induce mitochondrial damage. GSK3β, which is known to bind to α-syn, contributes to tau phosphorylation in cooperation with MAPKs. FIG. 26C. Pα-syn*/ptau aggregates induce mitochondrial fragmentation and recruit parkin, initiating mitophagy. FIG. 26D. Mitophagic vacuoles contain mitochondrial debris, along with Pα-syn*/ptau/MAPK aggregates.

FIG. 27: Pα-syn* accumulation in PFF-treated human dopaminergic neurons induces loss of dendritic spines. Reduced levels of pα-syn* are linked to neuroprotection and preservation of dendritic spines. Dopaminergic neurons differentiated for 30 days from neural stem cells were seeded with 50 μg/ml PFFs, and treated or not with 2 μM of a neuroprotective compound for 17 days. FIG. 27A: Phalloidin-iFluor 488 (Abcam) was used to label the dendritic spine marker F-actin (green); FIG. 27B: pα-syn* labeling with antibody GTX50222 (GeneTex, red) and DAPI (blue). Quantification shown on the right was done using ImageJ (NIH), statistical analysis with one way ANOVA (Prism7.04). Mean values and SDs of 8 images (A) or 6 images (B) per each condition are shown. ****P<0.0001; ***P<0.001; ns=non significant.

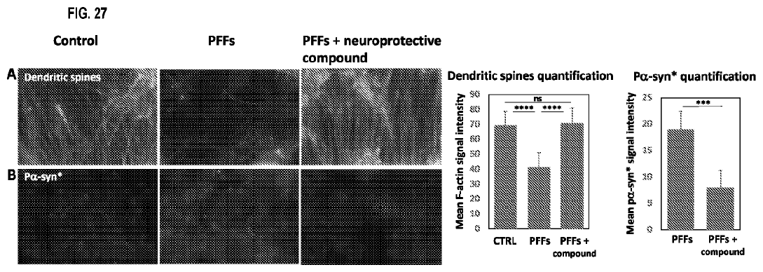

### DETAILED DESCRIPTION

Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). In addition, the following definitions are provided to assist the reader in the practice of the invention.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used in this specification and the appended claims, the term “or” is generally employed in its sense including “and/or” unless the content clearly dictates otherwise. Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The term “agent” is used to describe a compound that has or may have a therapeutic or pharmacological activity. Agents include compounds that are known drugs, compounds for which therapeutic activity has been identified but which are undergoing further therapeutic evaluation, and compounds that are members of collections and libraries that are to be screened for a pharmacological activity.

Mitotoxicity is meant to designate toxic activities for the mitochondria.

Pα-syn* refers to a conformationally distinct and nonfibrillar α-syn species with mitotoxic activities (e.g., inducing mitochondrial dysfunction with loss of membrane potential and structural damage resulting in mitochondrial fragmentation and mitophagy). Pα-syn* is also associated with synaptic toxicity, with the formation of pACC aggregates, with the phosphorylation of GSK3β and MAPKs MKK4, JNK, p38 and ERK5 and the formation of small phosphorylated tau aggregates, typically in the vicinity of the mitochondria. Typically, it contains phosphorylated residue S¹²⁹. α-Synuclein (α-syn) is a presynaptic neuronal protein, which in human is made of 140 amino acid residues and is encoded by the SNCA gene. In the brains of patients with PD or other synucleinopathies, a significant amount of α-syn is phosphorylated at residue S129 (Pα-syn). In some embodiments, Pα-syn* can exist in the triton X100 soluble and insoluble fractions as a monomer of about 12.5 kDa, a dimer of about 25 kDa and/or as larger oligomers. In some embodiments, Pα-syn* is a toxic variant of phosphorylated α-Synuclein (Pα-syn) harboring a specific conformation that is recognized by anti-phospho-Ser129 antibody GTX50222 (Landrock et al., Brain Res. 1679:155-170, 2018), which is available from GeneTex, Inc. (Irvine, Calif.). Additionally or alternatively, the Pα-syn* immunogen in these embodiments of the invention is not immunoreactive with antibodies 81A (Volpicelli-Daley et al., Nat. Protoc. 9: 2135-2146, 2014) and/or antibody MJF-R13 (Nelson et al., Acta. Neuropathol. Commun. 2:20, 2014) that specifically recognize fibrillar Pα-synF, which are available from commercial vendors such as BioLegend (San Diego, Calif.) and Abcam (Cambridge, Mass.). In some embodiments, Pα-syn* can encompass N- and C-terminally truncated species of Pα-syn as described herein. It may contain truncations of ≤25 residues at the C-terminus and ≤25 residues at the N-terminus of Pα-syn. In some of these embodiments, the α-syn derived polypeptide immunogen is immunoreactive with anti-phospho-Ser129 antibody GTX50222. Additionally or alternatively, the α-syn derived immunogen in these embodiments is not immunoreactive with fibrillar Pα-synF-recognizing 81A and/or antibody MJF-R13.

The term “adjuvant” refers to a compound that when administered in conjunction with an immunogen augments the immune response to the antigen, but when administered alone does not generate an immune response to the antigen. Adjuvants can augment an immune response by several mechanisms including lymphocyte recruitment, stimulation of B and/or T cells, and stimulation of macrophages.

Competition between antibodies is determined by an assay in which the immunoglobulin under test inhibits specific binding of a reference antibody to a common antigen, such as α-syn. Numerous types of competitive binding assays are known, for example: solid phase direct or indirect radioimmunoassay (RIA), solid phase direct or indirect enzyme immunoassay (EIA), sandwich competition assay; solid phase direct biotin-avidin EIA; solid phase direct labeled assay, solid phase direct labeled sandwich assay; solid phase direct label RIA using 1-125 label; solid phase direct biotin-avidin EIA; and direct labeled RIA. Typically, such an assay involves the use of purified antigen bound to a solid surface or cells bearing either of these, an unlabeled test immunoglobulin and a labeled reference immunoglobulin. Competitive inhibition is measured by determining the amount of label bound to the solid surface or cells in the presence of the test immunoglobulin. Usually the test immunoglobulin is present in excess. Antibodies identified by competition assay (competing antibodies) include antibodies binding to the same epitope as the reference antibody and antibodies binding to an adjacent epitope sufficiently proximal to the epitope bound by the reference antibody for steric hindrance to occur. Usually, when a competing antibody is present in excess, it will inhibit specific binding of a reference antibody to a common antigen by at least 50 or 75%.

The term “antibody” also synonymously called “immunoglobulins” (Ig), including antibody fragments described herein, refers to polypeptide chain(s) which exhibit a strong monovalent, bivalent or polyvalent binding to a given antigen, epitope or epitopes. Unless otherwise noted, antibodies or antigen-binding fragments used in the invention can have sequences derived from any vertebrate species. They can be generated using any suitable technology, e.g., hybridoma technology, ribosome display, phage display, gene shuffling libraries, semi-synthetic or fully synthetic libraries or combinations thereof. Unless otherwise noted, the term “antibody” as used in the present invention includes intact antibodies, antigen-binding polypeptide fragments and other designer antibodies that are described below or well known in the art.

An intact “antibody” typically comprises at least two heavy (H) chains (about 50-70 kD) and two light (L) chains (about 25 kD) inter-connected by disulfide bonds. The recognized immunoglobulin genes encoding antibody chains include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

Each heavy chain of an antibody is comprised of a heavy chain variable region (V_{H}) and a heavy chain constant region. The heavy chain constant region of most IgG isotypes (subclasses) is comprised of three domains, C_{H1}, C_{H2} and C_{H3}, some IgG isotypes, like IgM or IgE comprise a fourth constant region domain, C_{H4} Each light chain is comprised of a light chain variable region (V_{L}) and a light chain constant region. The light chain constant region is comprised of one domain, C_{L}. The variable regions of the heavy and light chains contain a binding domain that interacts with an antigen. The constant regions of the antibodies may mediate the binding of the immunoglobulin to host tissues or factors, including various cells of the immune system and the first component (C1q) of the classical complement system.

The term “combination therapy”, as used herein, refers to those situations in which two or more different agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

A “conservative substitution” with respect to proteins or polypeptides refers to replacement of one amino acid with another amino acid having a similar side chain. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Methods of identifying nucleotide and amino acid conservative substitutions which do not eliminate protein activity are well-known in the art (see, e.g., Brummell et ah, Biochem. 32: 1180-1187 (1993); Kobayashi et ah, Protein Eng. 12(10):879-884 (1999); and Burks et al, Proc. Natl. Acad. Sci. USA 94:.412-417 (1997)).

The term “contacting” has its normal meaning and refers to combining two or more agents (e.g., polypeptides or phage), combining agents and cells, or combining two populations of different cells. Contacting can occur in vitro, e.g., mixing an antibody and a cell or mixing a population of antibodies with a population of cells in a test tube or growth medium. Contacting can also occur in a cell or in situ, e.g., contacting two polypeptides in a cell by co-expression in the cell of recombinant polynucleotides encoding the two polypeptides, or in a cell lysate. Contacting can also occur in vivo inside a subject or a non-human animal, e.g., by administering an agent to a subject for delivery the agent to a target cell.

The terms “determining”, “measuring”, “evaluating”, “detecting”, “assessing” and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The terms “identical” or percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

Methods of alignment of sequences for comparison are well known in the art. Optimal alignment of sequences for comparison can be conducted, e.g., by the local homology algorithm of Smith and Waterman, Adv. Appl. Math. 2:482c, 1970; by the homology alignment algorithm of Needleman and Wunsch, J. Mol. Biol. 48:443, 1970; by the search for similarity method of Pearson and Lipman, Proc. Nat'l. Acad. Sci. USA 85:2444, 1988; by computerized implementations of these algorithms (GAP, BESTFIT, FASTA, and TFASTA in the Wisconsin Genetics Software Package, Genetics Computer Group, Madison, Wis.); or by manual alignment and visual inspection (see, e.g., Brent et al., Current Protocols in Molecular Biology, John Wiley & Sons, Inc. (ringbou ed., 2003)). Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively.

The term “modulator” is used to refer to an entity whose presence in a system in which an activity of interest is observed correlates with a change in level and/or nature of that activity as compared with that observed under otherwise comparable conditions when the modulator is absent. In some embodiments, a modulator is an activator, in that activity is increased in its presence as compared with that observed under otherwise comparable conditions when the modulator is absent. In some embodiments, a modulator is an inhibitor, in that activity is reduced in its presence as compared with otherwise comparable conditions when the modulator is absent. In some embodiments, a modulator interacts directly with a target entity whose activity is of interest. In some embodiments, a modulator interacts indirectly (i.e., directly with an intermediate agent that interacts with the target entity) with a target entity whose activity is of interest. In some embodiments, a modulator affects level of a target entity of interest; alternatively or additionally, in some embodiments, a modulator affects activity of a target entity of interest without affecting level of the target entity. In some embodiments, a modulator affects both level and activity of a target entity of interest, so that an observed difference in activity is not entirely explained by or commensurate with an observed difference in level.

The phrase “pharmaceutically acceptable carrier” refers to a carrier for the administration of a therapeutic agent. Exemplary carriers include saline, buffered saline, dextrose, water, glycerol, ethanol, and combinations thereof. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

Synucleinopathies (also called α-Synucleinopathies) are neurodegenerative diseases characterized by the abnormal accumulation of aggregates of alpha-synuclein protein in neurons, nerve fibers or glial cells. They are characterized by degeneration of the dopaminergic system and other areas of the central nervous system. They manifest clinically with motor alterations, cognitive impairment, autonomous dysfunction and neuropathologically with the formation of alpha-synuclein aggregates, sometimes in the form of Lewy bodies (LBs). Synucleinopathies include Parkinson's disease (PD), dementia with Lewy bodies (DLB), Lewy body variant of Alzheimer's disease, combined Parkinson's disease (PD) and Alzheimer's disease (AD), and multiple system atrophy (MSA).

A therapeutic activity refers to the activity of an agent that is or may be useful in the prophylaxis or treatment of a disease. The screening system can be in vitro, cellular, animal or human. Agents can be described as having therapeutic activity notwithstanding that further testing may be required to establish actual prophylactic or therapeutic utility in treatment of a disease.

The phrases “specifically binds” refers to a binding reaction which is determinative of the presence of the protein in the presence of a heterogeneous population of proteins and other biologies. Thus, under designated conditions, a specified ligand binds preferentially to a particular protein and does not bind in a significant amount to other proteins present in the sample. A molecule such as antibody that specifically binds to a protein often has an association constant of at least 10⁶M or 10⁷ M⁻¹ preferably 10⁸M−¹ to 10⁹ M⁻¹, and more preferably, about 10¹⁰ M⁻¹ to 10¹¹ M⁻¹ or higher. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

Immunogens or therapeutic agents of the invention are typically substantially pure from undesired contaminant. This means that an agent is typically at least about 50% w/w (weight/weight) purity, as well as being substantially free from interfering proteins and contaminants. Sometimes the agents are at least about 80% w/w and, more preferably at least 90 or about 95% w/w purity. However, using conventional protein purification techniques, homogeneous peptides of at least 99% w/w can be obtained.

By “subject” is meant an organism to which the methods of the invention can be applied and/or to which the agents of the invention can be administered. A subject can be a mammal, including a human, or a mammalian organ or mammalian cells, including a human organ and/or human cells.

Certain methodologies of the instant invention include a step that involves comparing a value, level, feature, characteristic, property, etc. to a “suitable control”, referred to interchangeably herein as an “appropriate control”. A “suitable control” or “appropriate control” is a control or standard familiar to one of ordinary skill in the art useful for comparison purposes. In one embodiment, a “suitable control” or “appropriate control” is a value, level, feature, characteristic, property, etc. determined prior to performing a treatment and/or agent administration methodology, as described herein. For example, a transcription rate, mRNA level, translation rate, protein level, biological activity, cellular characteristic or property, genotype, phenotype, etc. can be determined prior to introducing a treatment and/or agent of the invention to a subject. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

“Treating” or “treatment” covers the treatment of a disease-state in a mammal, and includes: (a) preventing the disease-state from occurring in a mammal, in particular, when such mammal is predisposed to the disease-state but has not yet been diagnosed as having it; (b) inhibiting the disease-state, e.g., arresting it development; and/or (c) relieving the disease-state, e.g., causing regression of the disease state until a desired endpoint is reached. Treating also includes the amelioration of a symptom of a disease (e.g., lessen the pain or discomfort), wherein such amelioration may or may not be directly affecting the disease (e.g., cause, transmission, expression, etc).

A “vector” is a replicon, such as plasmid, phage or cosmid, to which another polynucleotide segment may be attached so as to bring about the replication of the attached segment. Vectors capable of directing the expression of genes encoding for one or more polypeptides are referred to as “expression vectors”.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. Concentrations, amounts, cell counts, percentages and other numerical values may be presented herein in a range format. It is to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

II. Overview

Exposure of cultured primary neurons to preformed α-synuclein fibrils (PFFs) leads to the recruitment of endogenous α-synuclein and its templated conversion into fibrillar phosphorylated α-synuclein (Pα-synF) aggregates, resembling those involved in Parkinson's disease (PD) pathogenesis. Pα-synF was described previously as inclusions morphologically similar to Lewy bodies and Lewy neurites in PD patients. The instant invention is predicated in part on the discovery by the present inventors of the existence of a conformationally distinct, non-fibrillar, phosphorylated α-syn species capable of inducing mitochondrial dysfunction and structural damage resulting in mitophagy, formation of pACC and ptau aggregates, phosphorylation of several enzymes of the MAPK pathway as well as GSK3β, and loss of dendritic spines, which is termed Pα-syn* herein. As detailed below, Pα-syn* was found to be present in PFF-seeded primary neurons, mice brains and PD patients brains. Through immunofluorescence and pharmacological manipulation, it was observed that Pα-syn* results from incomplete autophagic degradation of Pα-synF. Pα-synF was decorated with autophagic markers, but not Pα-syn*. In some experimental conditions, western blots revealed Pα-syn* migrating at 12.5 kDa, possibly resulting from N- and/or C-terminal trimming of α-syn, and as a SDS-resistant dimer. After lysosomal release, Pα-syn* aggregates associated with mitochondria and induced mitochondrial membrane depolarization, cytochrome C release and mitochondrial fragmentation as visualized by STED nanoscopy. Pα-syn* induced the formation of small aggregates of phosphorylated acetyl-CoA carboxylase (ACC) with which it remarkably colocalized. ACC is the enzyme that catalyzes the synthesis of malonyl-CoA, the first committed step in the synthesis of fatty acids. ACC phosphorylation indicates low ATP levels, AMPK activation, oxidative stress. ACC phosphorylation reduces the activity of ACC, resulting in decreased de novo fatty acid synthesis, and mitochondrial fragmentation with reduced lipoylation. In addition, Pα-syn* also colocalized with BiP, a master regulator of the unfolded protein response (UPR), and resident protein of mitochondria associated ER membranes (MAMs) that are sites of mitochondrial fission and mitophagy. Further, Pα-syn* aggregates were found in Parkin positive mitophagic vacuoles and imaged by electron microscopy. Pα-syn* aggregates were found to induce and co-localize with phosphorylated MAPKs (MKK4, JNK, p38, ERK5) and GSK3β, as well as small aggregates of phosphorylated tau. pTau aggregates were co-localizing with Pα-syn* at the mitochondrial membrane, especially in areas of fragmented mitochondria. Elevated Pα-syn* levels correlated with a reduction in dendritic spines, and reduced Pα-syn* levels induced by compound treatment correlated with an increase in the number of dendritic spines used to quantify synaptic health. These findings indicate that Pα-syn* induces mitochondrial toxicity and fission, energetic stress, alteration of lipid metabolism, mitophagy, kinase activation, formation of ptau aggregates and synaptic toxicity, implicating Pα-syn* as a key neurotoxic α-syn species and new therapeutic target.

In accordance with these studies, the invention provides methods of using Pα-syn* and related polypeptides as immunogen or assay marker in various pharmaceutical and industrial applications. As detailed below, the invention provides methods of employing Pα-syn* to generate antibodies that can be useful for treating and/or diagnosing PD and other synucleinopathies. The invention also provides methods of using Pα-syn* as a marker to screen for novel therapeutic agents for treating PD and other synucleinopathies and as a biomarker for disease state and/or disease progression in PD and other synucleinopathies. As described herein, Pα-syn* can exist in both soluble and insoluble form. It can be derived from human α-syn or α-syn from other species (e.g., mouse). Such immunogen can be readily obtained based on the present disclosure. For example, as detailed herein, fibrils of recombinant α-syn (preformed fibrils or PFFs) can be used to seed the misfolding and aggregation of endogenous α-syn in cell lines and primary neurons, leading to the formation of large triton-insoluble α-syn fibrils. These fibrils are composed of α-syn phosphorylated at S129 (Pα-syn), mimicking the formation of LB s in PD patients brains where >90% of α-syn is phosphorylated at S129. PFF-seeded neurons also produce Pα-syn*. They undergo autophagic and metabolic failure, mitochondrial pathology, defective vesicular transport, synaptic dysfunction and neuronal death. Pα-syn* can then be isolated from the cell culture, for example by immunoprecipitation, electrophoresis of cell lysates and gel extraction, chromatography or other protein purification methods that can be used by one skilled in the art. Brain extracts from animal models of synucleinopathies or patients affected by a synucleinopathy can also be used to seed neuronal cultures. Other than seeded primary neurons, the Pα-syn* immunogen can also be isolated from mice brains that have been injected with recombinant α-synuclein fibrils, brain extracts from animal models of synucleinopathies, or brains of patients affected by a synucleinopathy.

In some other embodiments, the Pα-syn* polypeptide immunogen to be used in the practice of the invention can be generated in vitro, e.g., by recombinant expression or by chemical synthesis. In some preferred embodiments, the α-syn derived immunogen or polypeptide to be used in the methods of the invention can be produced recombinantly. In the practice of the methods of the invention, the recombinantly produced α-syn immunogen can be either phosphorylated (Pα-syn*) or non-phosphorylated (α-syn*). In vitro phosphorylation of recombinantly produced α-syn fragments can be performed as described in the art, e.g., Schreurs et al., Int. J. Mol. Sci. 15:1040-67, 2014; and Lu et al., ACS Chem. Neurosci., 2011, 2:667-675, 2011. Sequences of alpha-synuclein of human and many other species are well known and characterized in the art. These include α-syn amino acid sequences and their encoding cDNA sequences. See, e.g., GenBank Accession Nos:CR541653.1 and CAG46454.1; Ueda et al., Proc. Natl. Acad. Sci. U.S.A. 90:11282-11286, 1993; Campion et al., Genomics 26:254-257, 1995; and Touchman et al., Genome Res. 11:78-86, 2001. For example, the amino acid sequence of a human Pα-syn isoform (accession no. NP_001139526) is shown below:

mdvfmkglsk akegvvaaae ktkqgvaeaa gktkegvlyv gsktkegvvh gvatvaektk eqvtnvggav vtgvtavaqk tvegagsiaa atgfvkkdql gkneegapqe giledmpvdp dneayempse egyqdyepea (SEQ ID NO:1).

In some embodiments, the Pα-syn* immunogen or its non-phosphorylated counterpart (α-syn*) is derived from α-syn with truncations at the N-terminus and/or the C-terminus. Relative to the full length wildtype α-syn protein, the recombinantly produced immunogen can have (1) an N-terminal deletion of about 1, 2, 3, 4, 5 , 6, 7, 8, 9, 10, 11, 12, 13, 14, 15, 16, 17, 18, 19, 20, 21, 22, 23, 24 or 25 residues and/or (2) a C-terminal deletion of about 1, 2, 3, 4, 5 , 6, 7, 8, 9, 10, 11, 12, 13, 14, 15, 16, 17, 18, 19, 20, 21, 22, 23, 24 or 25 residues. In some embodiments, the Pα-syn* (or α-syn*) immunogen contains truncations of the first 3, 4, 5, 6, 7, 8, 9, or 10 N-terminal residues of the full length α-syn protein. In some other embodiments, the immunogen contains truncations of the first 11, 12, 13, 14, 15, 16, 17 or 18 N-terminal residues of the full length α-syn protein. In some other embodiments, the immunogen contains truncations of the first 19, 20, 21, 22, 23, 24, or 25 N-terminal residues of the full length α-syn protein. In addition to the N-terminal truncations, the immunogen can alternatively or additionally contain truncation of the first 3, 4, 5, 6, 7, 8, 9, or 10 C-terminal residues. In some other embodiments, the C-terminal truncation of the immunogen constitutes deletion of the first 11, 12, 13, 14, 15, 16, 17 or 18 C-terminal residues. In some other embodiments, the C-terminal truncation of the immunogen constitutes deletion of the first 19, 20, 21, 22, 23, 24, or 25 C-terminal residues. In various embodiments, the Pα-syn* or α-syn* immunogen can have a combination of a N-terminal truncation of from 1 to about 25 residues and a C-terminal truncation of from 1 to about 25 residues. For example, in some embodiments, the Pα-syn* or α-syn* immunogen has truncations of (1) the first 13, 14, or 15 N-terminal residues and (2) the first 8, 9 or 10 C-terminal residues of the full length α-syn protein. In some embodiments, the Pα-syn* or α-syn* immunogen contains truncations of the first 15 N-terminal residues and the first 10 C-terminal residues of the full length α-syn protein. In some of these embodiments, the full length α-syn protein constitutes an amino acid sequence as shown in SEQ ID NO:1. In some embodiments, the polypeptide immunogen is encoded by the nucleic acid sequence of SEQ ID NO: 1 or a variant thereof having at least about 50% (such as at least about any of 55%, 60%, 65%, 70%, 75%, 80%, 85%, 86%, 87%, 88%, 89%, 90%, 91%, 92%, 93%, 94%, 95%, 96%, 97%, 98%, 99%, or greater) sequence identity to SEQ ID NO: 1.

In addition to human a,-syn sequence exemplified herein, Pα-syn* or its non-phosphorylated counterpart (α-syn*) suitable for the invention can also be derived from ot-syn analogs including allelic, species and induced variants. Relative to the wildtype α-syn sequence, the variant can contain an amino acid sequence that is different at one, two or a few positions, often by virtue of conservative substitutions. For example, the variant can contain the A30P and/or A53T substitutions. In some embodiments, the variants contain a sequence that is substantially identical to the naturally occurring α-syn sequence SEQ ID NO:1). In some embodiments, the variants contain one or more unnatural amino acid residues. When a non-naturally existing or a non-human α-syn sequence is employed in the practice in the invention, their amino acid residues are assigned the same numbers as corresponding amino acids in the natural human sequence when the analog and human sequence are maximally aligned. Thus, for example, a phosphorylated Ser¹²⁹ residue in a truncated α-syn sequence refers to the residue that is numbered according to human α-syn sequence (e.g., SEQ ID NO:1), i.e., the residue corresponding to residue Ser¹²⁹ in the human α-syn sequence. In still some other embodiments, a synthetic polymer mimicking the specific structure or conformation of Pα-syn* can be employed in lieu of Pα-syn* as an immunogen.

Various recombinant expression systems can be employed to produce the full-length or N-terminal and/or C-terminal truncated α-syn polypeptides described herein. For example, both viral-based and nonviral expression vectors can be used to produce the polypeptides in a mammalian host cell. Nonviral vectors and systems include plasmids, episomal vectors, typically with an expression cassette for expressing a protein or RNA, and human artificial chromosomes (see, e.g., Harrington et al., Nat. Genet. 15:345, 1997). For example, nonviral vectors useful for expression of the polypeptides in mammalian (e.g., human) cells include pCEP4, pREP4, pThioHis A, B & C, pcDNA3.1/His, pEBVHis A, B & C (Invitrogen, San Diego, Calif.), MPSV vectors, and numerous other vectors known in the art for expressing other proteins. Other useful nonviral vectors include vectors that comprise expression cassettes that can be mobilized with Sleeping Beauty, PiggyBack and other transposon systems. Useful viral vectors include vectors based on lentiviruses or other retroviruses, adenoviruses, adeno-associated viruses, herpes viruses, vectors based on SV40, papilloma virus, HBP Epstein Barr virus, vaccinia virus vectors and Semliki Forest virus (SFV). Expression in a host cell (e.g., HEK 293, CHO or insect cell lines) and purification of the polypeptides can be readily performed in accordance with methods routinely practiced in the art. See, Brent et al., supra; Smith, Annu. Rev. Microbiol. 49:807, 1995; Khan, Adv Pharm Bull., 3(2): 257-263, 2013; and Rosenfeld et al., Cell 68:143, 1992.

Unless otherwise indicated, the invention can employ conventional techniques of cell biology, cell culture, molecular biology, transgenic biology, microbiology, recombinant DNA, and immunology, which are within the skill of the art. Such techniques are explained fully in the literature. See, for example, Sambrook et al, ed. (1989) Molecular Cloning A Laboratory Manual (2nd ed.; Cold Spring Harbor Laboratory Press); Sambrook et al, ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Cabs eds. (1987) Gene Transfer Vectors For Mammalian Cells, (Cold Spring Harbor Laboratory); Wu et al, eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); and in Ausubel et al. (1989) Current Protocols in Molecular Biology (John Wiley and Sons, Baltimore, Md.).

III. Methods for Generating Novel Antibodies for Treating PD and Other Synucleinopathies

In one aspect, the invention provides methods for generating therapeutic agents (e.g., therapeutic antibodies) that specifically target Pα-syn*. Such agents are useful in the treatment of Parkinson's disease and synucleinopathies. There is currently no disease-modifying treatment for Parkinson's disease and synucleinopathies such as dementia with Lewy bodies, and multiple systems atrophy. Current efforts to develop reagents (in particular antibodies) are directed against Lewy Bodies/Lewy Neurites types of α-synuclein aggregates (similar to Pα-synF described herein) or the native, endogenous form of α-synuclein. As demonstrated herein, Pα-syn* is a better therapeutic target since this is the entity directly associating with mitochondria and inducing their damage. Mitochondrial damage, fission and mitophagy are known to be key to the death of dopaminergic neurons in Parkinson's disease, but a direct link between Lewy Bodies/Neurites and mitochondrial damage has never been established. Because Pα-syn* induces such mitochondrial damage, it represents a privileged target for the development of therapeutic agents that are capable of slowing or stopping the progression of Parkinson's disease and synucleinopathies.

Pα-syn* is also found to trigger phosphorylation of several kinases such as MAPKs and GSK3β, as well as the formation of small aggregates of phosphorylated ACC, a rate limiting enzyme in the synthesis of fatty acids. Therefore, targeting Pα-syn* is poised to prevent several pathogenic events. Moreover, Pα-syn* is found associated with phosphorylated Tau (pTau). Phosphorylated Tau is known to be another molecular player of neurodegeneration and found in conjunction with Pα-syn inclusions in Parkinson's disease, Dementia with Lewy Bodies, Lewy Body variant of Alzheimer's disease and Down syndrome. As described herein, Pα-syn* triggers the formation of pTau aggregates. Reduction of pTau provides another beneficial effect of Pα-syn* targeting. The importance of targeting certain types of smaller amyloid aggregates, rather than large ‘plaques’ or ‘fibrils’ is further exemplified by recent failures of clinical trials for Alzheimer's disease, involving the use of antibodies directed against Aβ amyloid plaques rather than smaller toxic Aβ aggregates.

Thus, some methods of the invention are directed to generating antibodies that are specific for Pα-syn*. In these methods, an immunogen composition containing a Pα-syn* or α-syn* polypeptide is used to immunize a non-human animal (e.g., mouse, rabbit or camel). In addition to the α-syn derived polypeptide, the immunogen composition may contain other additives that can enhance the immune response to the polypeptide. In some embodiments, the composition can contain one or more adjuvants. Typically, the adjuvants are mixed and injected together with the polypeptide immunogen into the animal. Examples of suitable adjuvants include complete Freund's adjuvant (CFA or ECA), incomplete Freund's adjuvant and solutions of aluminum hydroxide (alum). In some embodiments, the immunogen composition of the invention may also contain a carrier protein that helps elicit an immune response. Typically, the carrier protein is heterologous to α-syn, and is covalently or non-covalently conjugated to the Pα-syn* or related polypeptide immunogen. In some embodiments, the carrier protein is conjugated to a Pα-syn* immunogen which contains N-terminal and/or C-terminal truncations of the full length α-syn sequences as noted above. In these embodiments, other than the heterologous carrier protein, the Pα-syn* or related polypeptide in the immunogen composition of the invention is not linked to any N-terminal and/or C-terminal fragment sequence of a full length α-syn protein. In other words, the immunogen composition in these embodiments of the invention does not encompass a full length α-syn protein, or α-syn variants with an intact N-terminus and/or an intact C-terminus.

In some embodiments, the immunogen composition contains another polypeptide or a synthetic polymer which harbors a structural determinant identical to the conformational epitope conferring Pα-syn* its unique neurotoxic properties.

Polyclonal or monoclonal antibodies that are specific for Pα-syn* can be produced in accordance with standard techniques well known in the art of antibody engineering or specific protocols exemplified herein. For example, production of non-human monoclonal antibodies, e.g., murine, guinea pig, primate, rabbit or rat, can be performed as described in Harlow & Lane, Antibodies, A Laboratory Manual (CSHP NY, 1988). As routinely practiced in the art, complete Freund's adjuvant followed by incomplete adjuvant is preferred for immunization of laboratory animals. Rabbits or guinea pigs can be used for generating polyclonal antibodies. Mice can be used for producing monoclonal antibodies. Binding can be assessed, for example, by Western blot, ELISA or immunocytochemistry.

Antibodies specific for Pα-syn* include chimeric antibodies, humanized antibodies and human antibodies. Chimeric and humanized antibodies have the same or similar binding specificity and affinity as a mouse or other nonhuman antibody that provides the starting material for construction of a chimeric or humanized antibody. Chimeric antibodies are antibodies whose light and heavy chain genes have been constructed, typically by genetic engineering, from immunoglobulin gene segments beloninmg to different species. For example, the variable (V) segments of the genes from a mouse monoclonal antibody may be joined to human constant (C) segments, such as IgG1 and IgG4. Human isotype IgG1 is preferred. In some methods, the isotype of the antibody is human IgG1. IgM antibodies can also be used in some methods. A typical chimeric antibody is thus a hybrid protein consisting of the V or antigen-binding domain from a mouse antibody and the C or effector domain from a human antibody.

Humanized antibodies have variable region framework residues substantially from a human antibody and complementarity determining regions substantially from a mouse-antibody. See, Queen et al., Proc. Natl. Acad. Sci. USA 86:10029-10033 (1989), WO 90/07861, U.S. Pat. Nos. 5,693,762, 5,693,761, 5,585,089, 5,530,101, and Winter, U.S. Pat. No. 5,225,539. The constant region(s), if present, are also substantially or entirely from a human immunoglobulin. The human variable domains are usually chosen from human antibodies whose framework sequences exhibit a high degree of sequence identity with the murine variable region domains from which the CDRs were derived. The heavy and light chain variable region framework residues can be derived from the same or different human antibody sequences. The human antibody sequences can be the sequences of naturally occurring human antibodies or can be consensus sequences of several human antibodies. See Carter et at, WO 92/22653. Certain amino acids from the human variable region framework residues are selected for substitution based on their possible influence on CDR conformation and/or binding to antigen.

Human antibodies against Pα-syn* can also be generated in accordance with a number of techniques well known in the art. Some human antibodies are selected by competitive binding experiments, or otherwise, to have the same epitope specificity as a particular mouse antibody. Techniques for producing human antibodies include the trioma methodology of Oestberg et al, Hybridoma 2:361-367 (1983); Oestberg, U.S. Pat. No. 4,634,664; and Engleman et al, U.S. Pat. No. 4,634,666 (each of which is incorporated by reference in its entirety for all purposes), use of non-human transgenic mammals having transgenes encoding at least a segment of the human immunoglobulin locus as described by, e.g., Lonberg et al, WO93/1222, U.S. Pat. Nos. 5,877,397, 5,874,299, 5,814,318, 5,789,650, 5,770,429, 5,661,016, 5,633,425, 5,625,126, 5,569,825, 5,545,806, Nature 148, 1547-1553 (1994), Nature Biotechnology 14, 826 (1996), Kucherlapati, WO 91/10741(each of which is incorporated by reference in its entirety for all purposes) and phage display methods see, e.g., Dower el at, WO 91/17271 and McCafferty et al, WO 92/01047, U.S. Pat. Nos. 5,877,218, 5,871,907, 5,858,657, 5,837,242, 5,733,743 and 5,565,332.

Once antibodies that recognize the Pα-syn* immunogen are generated, they can be further examined for therapeutic activities useful for treating PD and other synucleinopathies. Any activities indicative of a potential therapeutic effect for these disorders can be examined in these assays. These include, e.g., a reduction of α-syn aggregation, a disruption of α-syn aggregates, a slowing of Pα-syn* and/or Pα-synF formation, a disappearance of Pα-syn* and/or Pα-synF. The activity to be monitored in the assays can also be an inhibition of any other mitotoxic activities in a PFF-seeded primary neuron or another cellular assay as exemplified herein, e.g., depolarization of the inner mitochondrial membrane, cytochrome C release, mitochondrial fragmentation, pACC recruitment in the form of small aggregates, and abnormal mitophagy. The activity to be monitored can also be a reduction in the formation of phosphorylated Tau (pTau). The activity to be monitored can also be a reduction in MKK4, JNK, p38, ERK5 or GSK3β phosphorylation. The activity to be monitored can also be a reduction in synaptic pathology and increase in dendritic spine density. Methods of using appropriate cells or animal models to assess such activities are described herein.

The antibodies/reagents generated can be used as diagnostic tools to diagnose preclinical and/or clinical PD or other synucleinopathies in body fluids and/or tissues, and/or to monitor disease progression, including during clinical trials. Pα-syn* can be used as a biomarker for disease severity in PD and other synucleinopathies.

The generated antibodies can also be further examined for specificity and selectivity for Pα-syn*. Various competitive and non-competitive binding assays can be employed in the methods. In some embodiments, specific binding to a labeled or immobilized Pα-syn* or Pot-syn* can be detected in the presence of unlabeled native u-syn protein or Pα-synF. In some embodiments, a known antibody recognizing Pα-syn* can be employed in a competitive assay to confirm Pα-syn* binding specificity of an identified antibody. In some embodiments, large libraries of antibodies can be screened simultaneously using phage display technique. In some embodiments, the isolated antibodies can be screened for selectivity for Pα-syn* over α-syn or other variants of Pα-syn, e.g., Pα-synF. As described herein, the mitotoxic Pα-syn* forms oligomeric aggregates as compared to fibrillary Pα-synF. Antibodies selective for Pα-syn* over α-syn or other variants such as Pα-synF will be more effective to counter the mitotoxic activity of Pα-syn* in the treatment of PD and other synucleinopathies. Selectivity of an identified antibody for Pα-syn* over α-syn or other variants such as Pα-synF can be readily examined via standard immunology techniques well known in the art or specifically exemplified herein, e.g., ELISA, western blot, or immunocytochemistry.

In certain embodiments, the antibodies comprise: polyclonal and monoclonal antibodies, camelids antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv, nanobody) fragments, genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Single domain antibodies can be engineered from single monomeric variable domains of either camelids' heavy-chain antibody (V_{H}H) or cartilaginous fishes' IgNAR (V_{NAR}). Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

In some embodiments, the antigen-binding domain is a humanized antibody of fragments thereof. A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g. , the antibody from which the CDR residues are derived), e.g. , to restore or improve antibody specificity or affinity

In some embodiments, the heavy and light chains of an antibody can be full-length or can be an antigen-binding portion (a Fab, F(ab′)2, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and/or a variable light chain region, such as scFvs.

The term “variable region” or “variable domain”, when used in reference to an antibody, such as an antibody fragment, refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Single-domain antibodies are antibody fragments comprising all or a portion of the heavy chain variable domain or all or a portion of the light chain variable domain of an antibody. In certain embodiments, a single-domain antibody is a human single-domain antibody.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.

General principles of antibody engineering are set forth in Borrebaeck, ed. (1995) Antibody Engineering (2nd ed.; Oxford Univ. Press). General principles of protein engineering are set forth in Rickwood et al, eds. (1995) Protein Engineering, A Practical Approach (IRL Press at Oxford Univ. Press, Oxford, Eng.). General principles of antibodies and antibody-hapten binding are set forth in: Nisonoff (1984) Molecular Immunology (2nd ed.; Sinauer Associates, Sunderland, Mass.); and Steward (1984) Antibodies, Their Structure and Function (Chapman and Hall, New York, N.Y.). Additionally, standard methods in immunology known in the art and not specifically described can be followed as in Current Protocols in Immunology, John Wiley & Sons, New York; Stites et al, eds. (1994) Basic and Clinical Immunology (8th ed; Appleton & Lange, Norwalk, Conn.) and Mishell and Shiigi (eds) (1980) Selected Methods in Cellular Immunology (W.H. Freeman and Co., NY).

Standard references setting forth general principles of immunology include Current Protocols in Immunology, John Wiley & Sons, New York; Klein (1982) J., Immunology: The Science of Self-Nonself Discrimination (John Wiley & Sons, NY); Kennett et al, eds. (1980) Monoclonal Antibodies, Hybridoma: A New Dimension in Biological Analyses (Plenum Press, NY); Campbell (1984) “Monoclonal Antibody Technology” in Laboratory Techniques in Biochemistry and Molecular Biology, ed. Burden et al, (Elsevier, Amsterdam); Goldsby et al, eds. (2000) Kuby Immunology (4th ed.; W.H. Freeman & Co.); Roitt et al. (2001) Immunology (6th ed.; London: Mosby); Abbas et al. (2005) Cellular and Molecular Immunology (5th ed.; Elsevier Health Sciences Division); Kontermann and Dubel (2001) Antibody Engineering (Springer Verlag); Sambrook and Russell (2001) Molecular Cloning: A Laboratory Manual (Cold Spring Harbor Press); Lewin (2003) Genes VIII (Prentice Hall, 2003); Harlow and Lane (1988) Antibodies: A Laboratory Manual (Cold Spring Harbor Press); Dieffenbach and Dveksler (2003) PCR Primer (Cold Spring Harbor Press).

IV. Other Methods Utilizing Pα-syn* or Related α-syn Variants

In addition to generating novel antibodies useful for treating PD and other synucleinopathies, Pα-syn* or its non-phosphorylated counterpart can also be used in obtaining other therapeutic agents. For example, it can be used as a marker or tool to screen for novel therapeutic compounds. It can also be employed to generate polymers rather than an antibody that recognize the conformational epitope conferring specificity to pα-syn*. In one aspect, the invention provides methods to identify agents with therapeutic activities that can be useful in treating PD and other synucleinopathies. In various embodiments, the screening methods of the invention can be performed in vitro, in cells or with transgenic animals. Preferably, a cell or animal model for PD and other synucleinopathies is employed in the methods. For example, the model can be cultured primary neurons or cell lines injected with preformed fibrils of α-syn (PFFs) as exemplified herein. The model can also be a transgenic animal displaying characteristics of PD or other synucleinopathy. Typically, the cell or animal model is contacted or administered with a plurality of candidate agents. The cell or animal is then examined for a cellular response or phenotype evidencing potential therapeutic activities. As noted above, various therapeutic activities may be examined in these methods of the invention. For example, the methods can involve testing candidate agents for ability to disrupt or inhibit the formation or mitotoxic activity of Pα-syn*. In some embodiments, the candidate agents can be screened for activity in inhibiting formation of Pα-syn*. As demonstrated herein, Pα-syn* can be formed by partial degradation of Pα-synF fibrils. In some embodiments, the candidate agents can be screened for activity in degrading Pα-syn*. In some embodiments, the candidate agents can be screened for binding to Pα-syn*. In some embodiments, the candidate agents can be screened for activity in preventing neurotoxic effects of Pα-syn* such as mitochondrial dysfunction and fragmentation, synaptic pathology, formation of pACC aggregates, MAPK or GSK3β phosphorylation, and formation of ptau aggregates at the mitochondria. In these methods, candidate agents (antibodies or other compounds such as small molecules) can be contacted with primary neurons or cell lines seeded with PFFs or engineered cells expressing an α-syn* polypeptide as described herein. Alternatively, the candidate agents can be administered to a transgenic animal expressing an α-syn* polypeptide as described herein. Effect of the candidate agents on conversion of Pα-synF into Pα-syn* can be readily monitored as exemplified herein. In some methods, a candidate compound that can substantially inhibit or slow Pα-syn* formation or induce Pα-syn* degradation is identified as a potential therapeutic agent for treating PD and other synucleinopathies. Detection and quantitation of Pα-syn* in these assays can be readily performed with antibodies that are selective for Pα-syn*. As exemplification, one antibody specifically recognizing Pα-syn*, but not Pα-synF, is rabbit anti-pS ¹²⁹ α-syn antibody GTX50222 lot 821505177 as described herein. Effect of the candidate agents on mitochondrial health, dendritic health, kinase activation, formation of pACC aggregates, formation of ptau aggregates, can be readily monitored as exemplified herein.

In some embodiments, agents identified in a cell model to possess such an activity can be further evaluated in secondary screens of animal models of PD or in clinical trials to determine activity against motor, behavioral, cognitive or other symptoms of the diseases. In some related embodiments, the invention also provides methods for assessing the effect of known drugs and other agents on treating PD and synucleinopathies. Similarly, these methods involve testing the known drugs or agents for their ability to disrupt or inhibit the formation or mitotoxic activity of Pα-syn*, the synaptotoxic activity of Pα-syn*, the kinase activation activity of Pα-syn*, or the formation of pACC or ptau aggregates induced by Pα-syn*. In certain embodiments, the candidate agents or potential therapeutic agents inhibit several activities or functions of the α-syn derived polypeptide in vivo or in vitro. In certain embodiments, the candidate or potential therapeutic agent inhibits α-syn derived polypeptide mediated formation of phosphorylated acetyl-CoA carboxylase (ACC) aggregates. In certain embodiments, the candidate or potential therapeutic agent inhibits α-syn derived polypeptide mediated phosphorylation of glycogen synthase kinase 3 beta (GSK3β) and mitogen-activated protein kinases (MAPKs) such as mitogen-activated protein kinase kinase 4 (MKK4), c-Jun N-terminal kinase (JNK), p38 and extracellular signal-regulated kinase 5 (ERK5). In certain embodiments, the candidate or potential therapeutic agent inhibits α-syn derived polypeptide mediated formation of phosphorylated tau aggregates. In certain embodiments, the candidate or potential therapeutic agent inhibits α-syn derived polypeptide mediated synaptic toxicity and loss of dendritic spines of neurons.

In certain embodiments, those potential therapeutic agents identified based on the screening assays are selected for testing their therapeutic activity. In certain embodiments, the therapeutic activity is inhibition of a toxic activity in a cellular model of synucleinopathy or a reduction in the generation and propagation of pathogenic phosphorylated α-syn.

Candidate/Test Agents: Various candidate agents can be employed in the screening methods of the invention, including any naturally existing or artificially generated agents. They can be of any chemistry class, such as antibodies, proteins, peptides, small organic compounds, saccharides, fatty acids, steroids, purines, pyrimidines, nucleic acids, and various structural analogs or combinations thereof. In some embodiments, the screening methods utilize combinatorial libraries of candidate agents. Combinatorial libraries can be produced for many types of compounds that can be synthesized in a step-by-step fashion. Such compounds include polypeptides, beta-turn mimetics, nucleic acids, polysaccharides, phospholipids, hormones, prostaglandins, steroids, aromatic compounds, heterocyclic compounds, benzodiazepines, oligomeric N-substituted glycines and oligocarbamates. Large combinatorial libraries of the compounds can be constructed by the encoded synthetic libraries (ESL) method described in Affymax, WO 95/12608, Aflymax, WO 93/06121, Columbia University, WO 94/08051, Pharmacopeia, WO 95/35503 and Scripps, WO 95/30642 (each of which is incorporated herein by reference for all purposes). Peptide libraries can also be generated by phage display methods. See, e.g., Devlin, WO 91/18980. In some methods, prior to examining their ability to disrupt or inhibit Pα-syn* formation in a cell or animal model, combinatorial libraries of candidate agents can be first examined for suitability by determining their capacity to bind to Pα-syn*.

Candidate agents include numerous chemical classes, though typically they are organic compounds including small organic compounds, nucleic acids including oligonucleotides, peptides or antibodies. Small organic compounds suitably may have e.g. a molecular weight of more than about 40 or 50 yet less than about 2,500. Candidate agents may comprise functional chemical groups that interact with proteins and/or DNA.

Candidate agents may be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of e.g. bacterial, fungal and animal extracts are available or readily produced.

Chemical Libraries: Developments in combinatorial chemistry allow the rapid and economical synthesis of hundreds to thousands of discrete compounds. These compounds are typically arrayed in moderate-sized libraries of small molecules designed for efficient screening. Combinatorial methods can be used to generate unbiased libraries suitable for the identification of novel compounds. In addition, smaller, less diverse libraries can be generated that are descended from a single parent compound with a previously determined biological activity.

A combinatorial chemical library is a collection of diverse chemical compounds generated by either chemical synthesis or biological synthesis, by combining a number of chemical “building blocks,” such as reagents. For example, a linear combinatorial chemical library, such as a polypeptide library, is formed by combining a set of chemical building blocks (amino acids) in a large number of combinations, and potentially in every possible way, for a given compound length (i.e., the number of amino acids in a polypeptide compound). Millions of chemical compounds can be synthesized through such combinatorial mixing of chemical building blocks.

A “library” may comprise from 2 to 50,000,000 diverse member compounds. Preferably, a library comprises at least 48 diverse compounds, preferably 96 or more diverse compounds, more preferably 384 or more diverse compounds, more preferably, 10,000 or more diverse compounds, preferably more than 100,000 diverse members and most preferably more than 1,000,000 diverse member compounds. By “diverse” it is meant that greater than 50% of the compounds in a library have chemical structures that are not identical to any other member of the library. Preferably, greater than 75% of the compounds in a library have chemical structures that are not identical to any other member of the collection, more preferably greater than 90% and most preferably greater than about 99%.

The preparation of combinatorial chemical libraries is well known to those of skill in the art. For reviews, see Thompson et al., Synthesis and application of small molecule libraries, Chem Rev 96:555-600, 1996; Kenan et al., Exploring molecular diversity with combinatorial shape libraries, Trends Biochem Sci 19:57-64, 1994; Janda, Tagged versus untagged libraries: methods for the generation and screening of combinatorial chemical libraries, Proc Natl Acad Sci USA. 91:10779-85, 1994; Lebl et al., One-bead-one-structure combinatorial libraries, Biopolymers 37:177-98, 1995; Eichler et al., Peptide, peptidomimetic, and organic synthetic combinatorial libraries, Med Res Rev. 15:481-96, 1995; Chabala, Solid-phase combinatorial chemistry and novel tagging methods for identifying leads, Curr Opin Biotechnol. 6:632-9, 1995; Dolle, Discovery of enzyme inhibitors through combinatorial chemistry, Mol. Divers. 2:223-36, 1997; Fauchere et al., Peptide and nonpeptide lead discovery using robotically synthesized soluble libraries, Can J. Physiol Pharmacol. 75:683-9, 1997; Eichler et al., Generation and utilization of synthetic combinatorial libraries, Mol Med Today 1: 174-80, 1995; and Kay et al., Identification of enzyme inhibitors from phage-displayed combinatorial peptide libraries, Comb Chem High Throughput Screen 4:535-43, 2001.

Other chemistries for generating chemical diversity libraries can also be used. Such chemistries include, but are not limited to, peptoids (PCT Publication No. WO 91/19735); encoded peptides (PCT Publication WO 93/20242); random bio-oligomers (PCT Publication No. WO 92/00091); benzodiazepines (U.S. Pat. No. 5,288,514); diversomers, such as hydantoins, benzodiazepines and dipeptides (Hobbs, et al., Proc. Nat. Acad. Sci. USA, 90:6909-6913 (1993)); vinylogous polypeptides (Hagihara, et al., J. Amer. Chem. Soc. 114:6568 (1992)); nonpeptidal peptidomimetics with .beta.-D-glucose scaffolding (Hirschmann, et al., J. Amer. Chem. Soc., 114:9217-9218 (1992)); analogous organic syntheses of small compound libraries (Chen, et al., J. Amer. Chem. Soc., 116:2661 (1994)); oligocarbamates (Cho, et al., Science, 261:1303 (1993)); and/or peptidyl phosphonates (Campbell, et al., J. Org. Chem. 59:658 (1994)); nucleic acid libraries (see, Ausubel, Berger and Sambrook, all supra); peptide nucleic acid libraries (see, e.g., U.S. Pat. No. 5,539,083); antibody libraries (see, e.g., Vaughn, et al., Nature Biotechnology, 14(3):309-314 (1996) and PCT/US96/10287); carbohydrate libraries (see, e.g., Liang, et al., Science, 274:1520-1522 (1996) and U.S. Pat. No. 5,593,853); small organic molecule libraries (see, e.g., benzodiazepines, Baum C&E News, January 18, page 33 (1993); isoprenoids (U.S. Pat. No. 5,569,588); thiazolidinones and metathiazanones (U.S. Pat. No. 5,549,974); pyrrolidines (U.S. Pat. Nos. 5,525,735 and 5,519,134); morpholino compounds (U.S. Pat. No. 5,506,337); benzodiazepines (U.S. Pat. No. 5,288,514); and the like.

Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd., Moscow, RU, 3D Pharmaceuticals, Exton, Pa., Martek Bio sciences, Columbia, Md., etc.).

The screening assays of the invention suitably include and embody, animal models, cell-based systems and non-cell based systems. Identified genes, variants, fragments, or oligopeptides thereof are used for identifying agents of therapeutic interest, e.g. by screening libraries of compounds or otherwise identifying compounds of interest by any of a variety of drug screening or analysis techniques. The gene, allele, fragment, or oligopeptide thereof employed in such screening may be free in solution, affixed to a solid support, borne on a cell surface, or located intracellularly. The measurements will be conducted as described in detail in the examples section which follows.

In some embodiments, a method of identifying candidate therapeutic agents comprises screening a sample containing the specific target molecule in a high-throughput screening assay.

In another embodiment, a method of identifying therapeutic agents comprises contacting: (i) a target molecule with a candidate therapeutic agent; determining whether (i) the agent modulates a function of the peptide or interaction of the peptide with a partner molecule; or (ii) the agent modulates expression and/or function of the nucleic acid sequence of the target as measured by the light emission assays embodied herein.

In another embodiment, a method of identifying candidate therapeutic agents for treatment of disease, comprises culturing an isolated cell expressing a target molecule, administering a candidate therapeutic agent to the cultured cell; correlating the target molecules expression, activity and/or function in the presence or absence of a candidate therapeutic agent as compared to control cells, wherein a drug is identified based on desirable therapeutic outcomes. For example, a drug which modulates levels of the target molecule whereby such levels are responsible for the disease state or the target molecule modulates the activity or amount of another molecule whether upstream or downstream in a pathway. In other examples the assays measure kinase activity. In other examples, the assay measure binding partners. In other examples, the assay measures amounts of candidate therapeutic agents which provide a desired therapeutic outcome.

Another suitable method for diagnosis and candidate drug discovery includes contacting a test sample with a cell expressing a target molecule, and detecting interaction of the test agent with the target molecule, an allele or fragment thereof, or expression product of the target molecule an allele or fragment thereof.

In another preferred embodiment, a sample, such as, for example, a cell or fluid from a patient is isolated and contacted with a candidate therapeutic molecule. The genes, expression products thereof, are monitored to identify which genes or expression products are regulated by the drug.

In another aspect, the invention provides methods for diagnosing or monitoring disease progression in subjects affected by PD and other synucleinopathies. Pα-syn* can be used as a biomarker for progression of disease in a human affected by a synucleinopathy or in an animal model of synucleinopathy. Indeed, since Pα-syn* is causally associated with a number of toxic events affecting mitochondrial and synaptic health and the amounts of Pα-syn* increase as cellular pathology progresses. Pα-syn* levels in the brain, other tissues and body fluids will predictably reflect disease progression in a human or animal, and measuring Pα-syn* levels can be used as a biomarker to monitor the therapeutic effect of disease-modifying treatments in clinical trials. These methods entail detecting and measuring in a biological sample (e.g., a tissue or body fluid sample) from the subjects the presence and/or amounts of Pα-syn* immunogen described herein or related variants exhibiting the conformation and toxicity specific for Pα-syn*. In some methods, the biological sample is obtained from the brain of the subject. In some embodiments, the Pα-syn* immunogen or variant examined is a α-syn variant with a deletion of about 0 to 25 N-terminal amino acid residues and/or a deletion of about 0 to 25 C-terminal amino acid residues of a full length α-syn protein. In some of these embodiments, the α-syn polypeptide to be detected and quantified further contains phosphorylated Ser¹²⁹. Detection and quantitation of Pα-syn* immunogen or variant in the biological sample can be readily performed in accordance with the techniques exemplified herein or protocols routinely practiced in the art.

The invention also provides engineered cells (e.g., neural cells) and transgenic animals expressing an α-syn construct prone to generate Pα-syn* or α-syn* as described above. The engineered cells and transgenic animal may be used in vitro or animal models to study PD and synucleinopathies as noted above, or to test the efficacy of therapeutic agents. The transgene is preferably present in all or substantially all of the somatic and germline cells of the transgenic animal. The polynucleotide encoding the full-length and/or mutated and/or truncated α-syn is operably linked to one or more regulatory segments that allow the α-syn variant to be expressed in neuronal cells of the animal. Promoters such as the rat neuron specific enolase promoter, the prion protein promoter, human beta-actin gene promoter, human platelet derived growth factor B (PDGF-B) chain gene promoter, rat sodium channel gene promoter, mouse myelin basic protein gene promoter, human copper-zinc superoxide dismutase gene promoter, and mammalian POU-domain regulatory gene promoter can be employed in expressing the transgene. Optionally, an inducible promoter can be used. The mouse metallothionine promoter, which can be regulated by addition of heavy metals such as zinc to the mouse's water or diet, is suitable. The engineered cells or transgenic animals of the invention can be produced by the general approaches described in the art, e.g., Masliah et al, Am. J. Pathol. 148:201-10, 1996; Feany et al, Nature 404:394-8, 2000; and U.S. Pat. No. 5,811,633.

### EXAMPLES

The following examples are provided to further illustrate the invention but not to limit its scope. Other variants of the invention will be readily apparent to one of ordinary skill in the art and are encompassed by the appended claims.

### Example 1

### Identification of Two Conformers of Pα-syn: Pα-syn* and Pα-synF

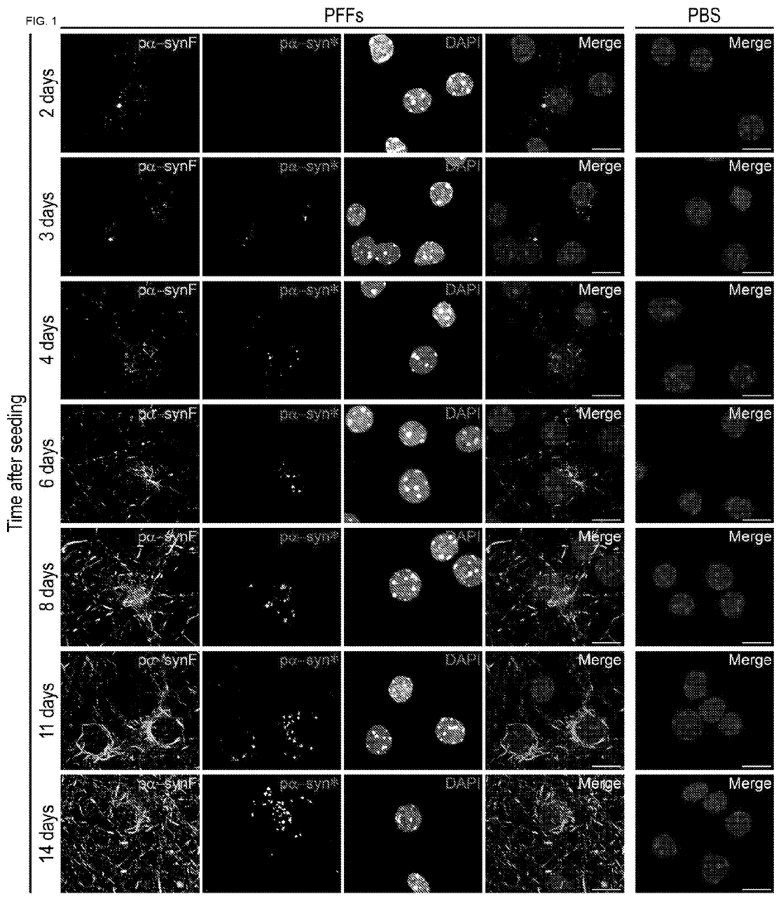

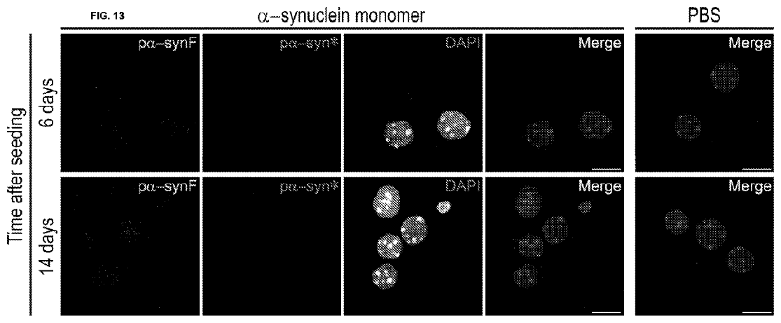

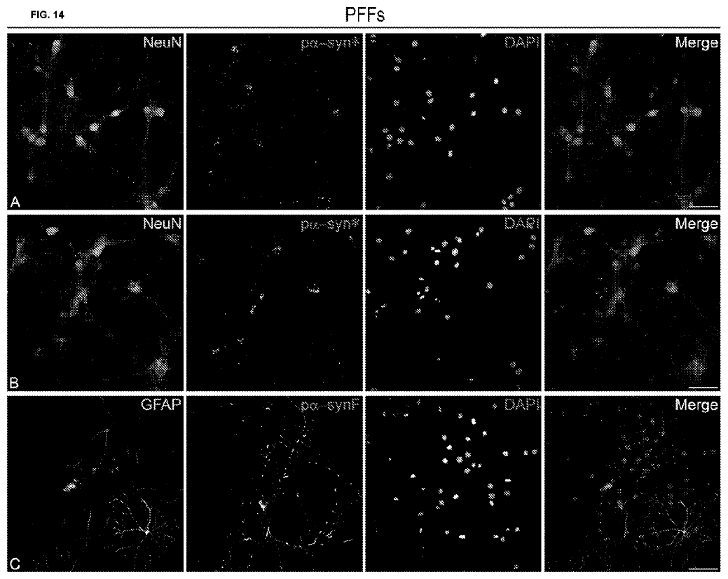

It was observed that seeding of primary neurons with preformed fibrils of α-syn (PFFs) leads to intracellular accumulation of two conformers of Pα-syn with different morphologies and cellular localization: Pα-syn* and Pα-synF. Using two different antibodies specific to phosphorylated S129 α-syn, we observed the existence of two distinct types of Pα-syn aggregates progressively accumulating over 14 days (FIG. 1). Neuronal demise prevented us from keeping the cultures past two weeks post-seeding. The first-to-appear and most abundant aggregates were visible as dot-like structures 2-3 days post-induction and rapidly formed elongated, bundled aggregates. This form of Pα-syn is known to be fibrillar and was named Pα-synF. As early as day 4, Pα-synF presented as long fibers similar to Lewy Neurites (LNs) described in PD patients, and, starting from day 6, as densely packed cage-like structures around the nucleus, always with a polarity, reminiscent of Lewy Bodies (LBs) (see FIG. 1, days 11 and 14). In addition, a less abundant, mostly punctate Pα-syn entity was also observed, progressively accumulating in areas densely packed with Pα-synF in the perinuclear region, and this distinctive form of Pα-syn was called Pα-syn*. Cells exposed to α-syn monomers instead of PFFs did not accumulate Pα-syn aggregates (FIG. 13). Pα-syn* was found to accumulate exclusively in neuronal cells (FIG. 14). Cultures were made of approximately 70% neuronal cells, with the vast majority of non-neuronal cells being astrocytes.

The strictly selective and mutually exclusive immunoreactivity of Pα-syn* and Pα-synF towards two different antibodies both recognizing an epitope comprising phosphorylated S129 of Pα-syn (polyclonal and monoclonal, respectively, as described in the methods), indicated that these aggregates represent different conformers of Pα-syn, and that the Pα-syn* antibody recognizes a conformational epitope. At this point, it was speculated that Pα-synF and Pα-syn* would neither share the same cellular interactome nor biological effects.

### Example 2

### Pα-syn* is also Found In Vivo in PFF-Injected Mice and PD Patients

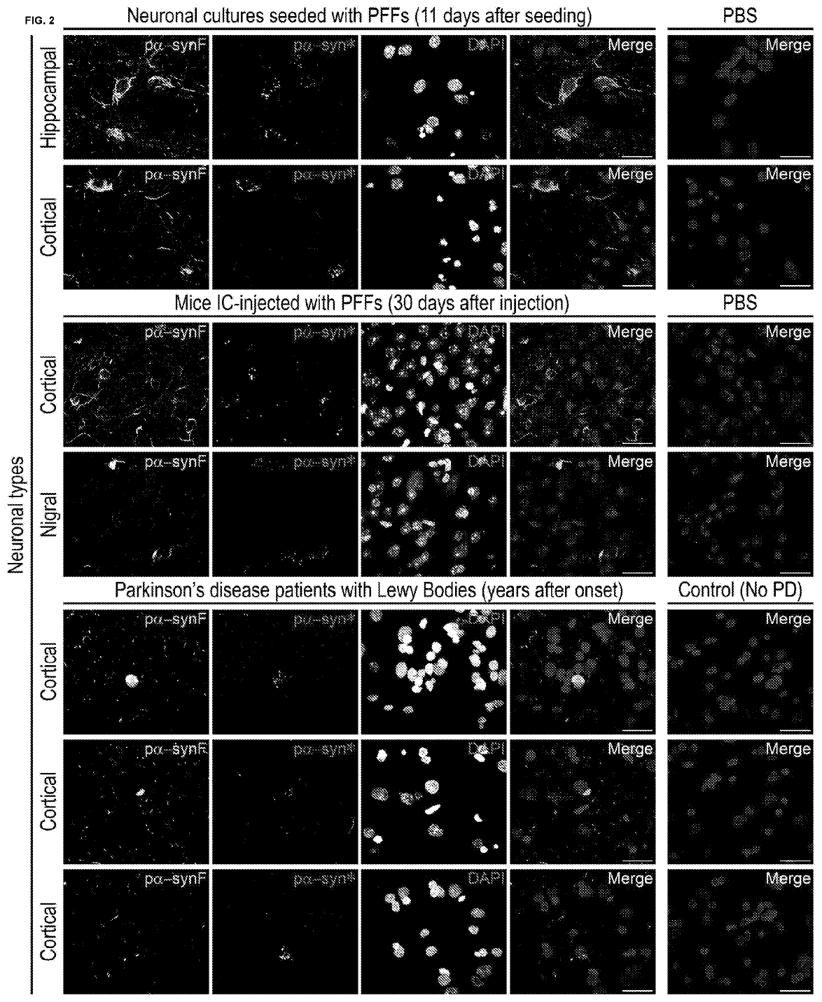

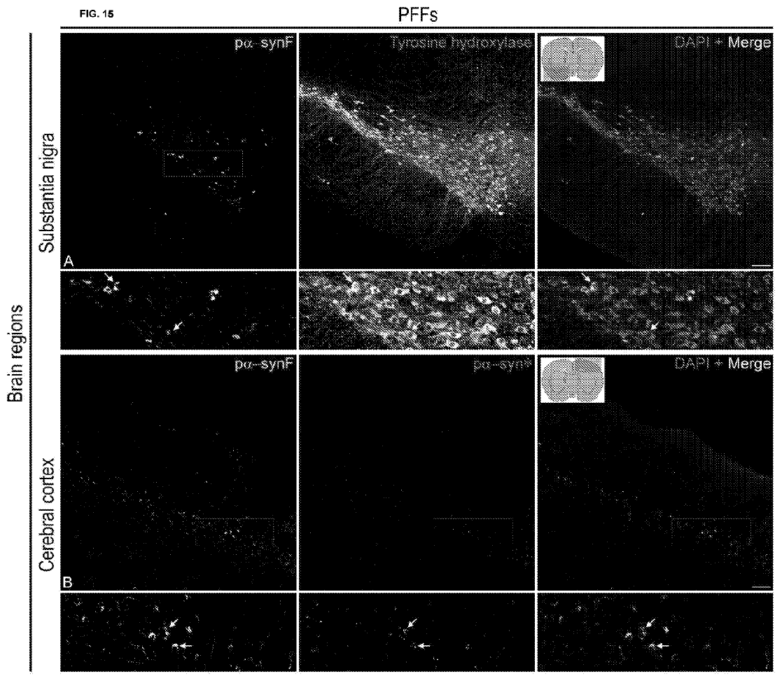

To establish the relevance of this finding in vivo, the brains of mice stereotaxically injected with PFFs and euthanized after 30 days were examined. Both Pα-synF and Pα-syn* inclusions were observed, with remarkably similar morphology and cellular localization as in the neuronal cultures. Despite the injection being made in the striatum, Pα-synF and Pα-syn* inclusions were present in both the cortex and the substantia nigra, showing appearance of pathogenic Pα-syn in brain regions that project to the striatum. They were more abundant in the cortex than in the substantia nigra (FIG. 2 and FIG. 15). Importantly, Pα-syn* conformers were also observed in brain sections of PD patients, showing the relevance of this Pα-syn species to human PD pathogenesis. Similar to the observations in neuronal cultures, Pα-syn* aggregates were generally observed in the vicinity of LBs in PD patients brains (FIG. 2). However, a quantitative relationship between the LB burden and the detection of Pα-syn* was not established. Patients' characteristics are presented in the Table 1.

TABLE 1

| Case ID | Condition | Gender | Expired ag | PMI | Unified LB Stage |
|---|---|---|---|---|---|
| 03-15 | Control | 1 | 8 | 3.2 | 0. No Lewy bodies |
| 03-28 | Control | 1 | 8 | 2.1 | 0. No Lewy bodies |
| 04-38 | Control | 2 | 8 | 4.8 | 0. No Lewy bodies |
| 05-12 | Control | 2 | 8 | 2 | 0. No Lewy bodies |
| 05-16 | Control | 1 | 8 | 3 | 0. No Lewy bodies |
| 06-15 | Control | 2 | 8 | 2. | 0. No Lewy bodies |
| 07-28 | Control | 1 | 8 | 4.7 | 0. No Lewy bodies |
| 07-37 | Control | 1 | 8 | 5. | 0. No Lewy bodies |
| 04-15 | PD (Low | 1 | 8 | 2.6 | lla. Brainstem |
| 04-17 | PD (Low | 1 | 8 | 2 | lla. Brainstem |
| 05-17 | PD (Low | 2 | 8 | 2 | lla. Brainstem |
| 03-48 | PD (Low | 1 | 9 | 3 | lla. Brainstem |
| 02-37 | PD (Low | 1 | 8 | 2.1 | llb. Limbic |
| 05-60 | PD (Low | 1 | 7 | 3. | lll. |
| 01-24 | PD (Low | 1 | 7 | 4. | lll. |
| 02-17 | PD (Low | 2 | 8 | 3 | lll. |
| 10-29 | PD (High | 1 | 7 | 2. | lV. Neocortical |
| 10-89 | PD (High | 2 | 8 | 3.2 | lV. Neocortical |
| 09-15 | PD (High | 1 | 8 | 3.7 | lV. Neocortical |
| 10-04 | PD (High | 1 | 8 | 3.5 | lV. Neocortical |
| 11-10 | PD (High | 2 | 8 | 3 | lV. Neocortical |
| 11-51 | PD (High | 2 | 8 | 1.9 | lV. Neocortical |
| 12-69 | PD (High | 2 | 6 | 2.7 | lV. Neocortical |
| 10-45 | PD (High | 1 | 8 | 3 | lV. Neocortical |

Gender, 1 = male, 2 = female; expired age is the individual's age at death; PMI, postmortem interval, the number of hours between death and brain removal; Unified LB Stage, Unified Lewy Body Staging score, 0 = no Lewy bodies, 1 = Lewy bodies present in the brainstem only, II = Lewy bodies present in the brainstem and limbic system, III = Lewy bodies present in the brainstem and limbic system and diffusely in the neocortex; IV = Lewy bodies present in the brainstem, limbic system and neocortex.

### Example 3

### Pα-syn* can Result from Incomplete Degradation of Pα-syn* Fibrils

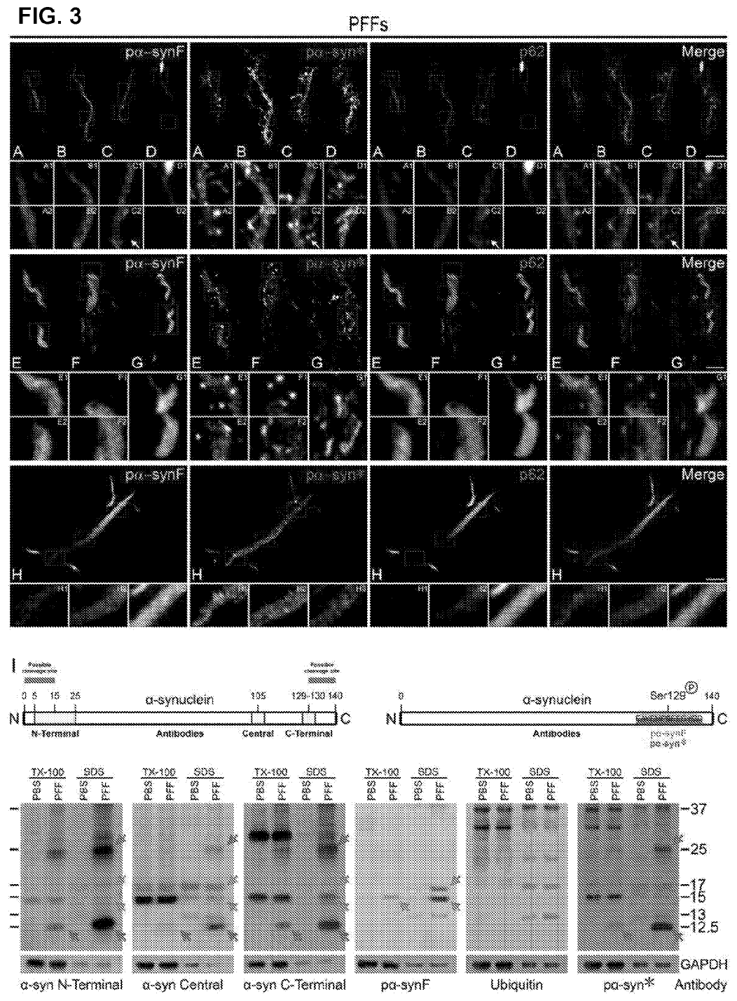

In the experimental system used, Pα-syn* resulted from incomplete degradation of Pα-synF fibrils. It was discovered that Pα-syn* originates from a conformational change within the Pα-synF fibrils. This conversion appeared to take place according to three scenarios, depending on the density and thickness of the original Pα-synF fibril. It is important to note that these fibrils have been shown to adopt a double stranded structure. The first scenario may be described as Pα-syn* shedding off of low density, thin Pα-synF fibrils (FIG. 3A-D). In this case, Pα-syn* appeared to be shed from the fibrils, along with a progressive disintegration of the Pα-synF core of the fibrils. Patches of Pα-syn* intermingled with patches of Pα-synF and ultimately, the fibrillar core completely disappeared and only newly generated Pα-syn* aggregates remained (FIG. 3D). The second scenario was observed with dense Pα-synF fibrils more clearly intertwined in a helical fashion. One strand of the helix unwound and was “digested” giving rise to Pα-syn* aggregates decorating the remaining strand (FIG. 3E-G). In a third scenario, dense fibers of Pα-synF appeared to undergo conversion into Pα-syn* from one end of the fiber, the conversion progressing towards the other end (FIG. 3H). In this case one end of the fibril exhibited Pα-synF immunoreactivity, the other end Pα-syn* immunoreactivity, and a degradation front was clearly observed.

Western blot analysis revealed that, in these experimental conditions, Pα-syn* could be detected as a truncated species of Pα-syn, migrating at 12.5 kDa (FIG. 3I, right panel, red arrows). Pα-syn* was detected specifically in PFF-treated neurons in both the triton X-100 soluble and insoluble fractions, presumably corresponding to the free and fibril-bound aggregates. Pα-syn* was also detected in a dimeric form at 25 kDa in the soluble and insoluble fractions of PFF-treated cells. In addition, the Pα-syn* antibody also detected basal levels of full-length phosphorylated α-syn (15 kDa band) present in the soluble fraction in both PBS- and PFF-treated cells. Both a C-terminal (encompassing aa 129-130) and a N-terminal α-syn antibody (epitope located within the first 25 aa of α-syn) were able to detect Pα-syn* (FIG. 3I, first three panels, red arrows). This shows that the truncated form of Pα-syn* detected in this experiment resulted from a loss of ≤25 Aa, and more likely ≤10 Aa at the C-terminus of Pα-syn and ≤25 Aa at the N-terminus (FIG. 3I—scheme). Of note, a Pα-synF antibody, while recognizing full-length Pα-syn (FIG. 3I fourth panel, green arrows), did not recognize the 12.5 kDa Pα-syn* species nor the 25 kDa dimer thereof, emphasizing the differential immunoreactivity between Pα-syn* and Pα-synF (FIG. 3).

Together, these morphological and biochemical data show that Pα-syn* can result from partial proteolytic degradation of Pα-synF fibrils. These data also illustrate the different immunoreactivity, hence the different conformational epitope, between Pα-syn* and Pα-synF.

### Example 4

### Pα-syn* is a Result of Incomplete Autophagic Degradation of Pα-synF

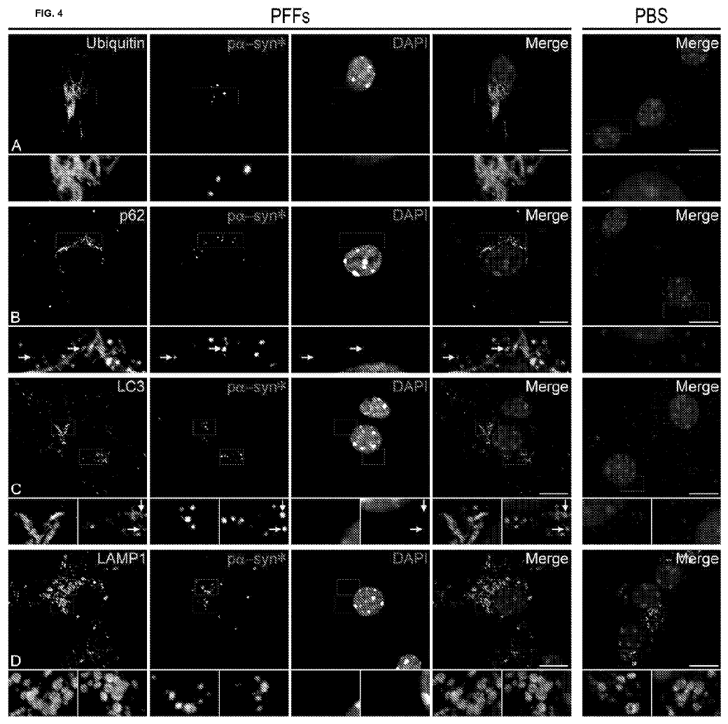

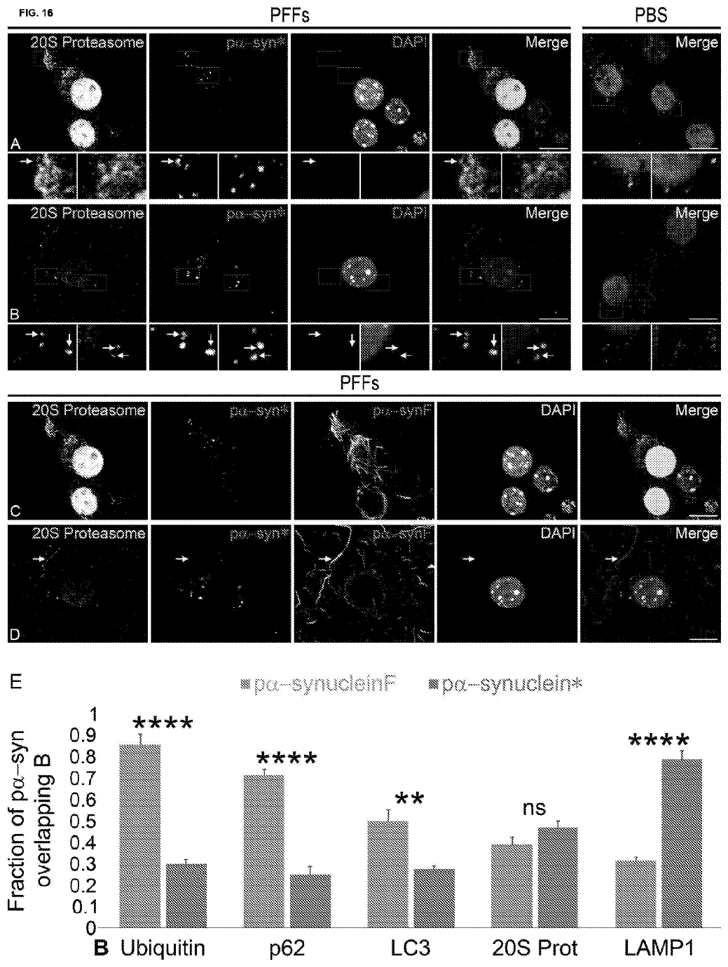

P62, an adaptor protein linking ubiquitinated protein aggregates to LC3 anchored in the membrane of the nascent autophagosome, labeled Pα-synF with remarkable congruence, but not Pα-syn* (FIG. 3A-H). This strongly suggested that Pα-synF fibrils were undergoing autophagy and that Pα-syn* may result from this proteolytic process. In order to confirm this hypothesis, PFF-treated cells were labeled with various markers recapitulating some major steps of autophagy. Ubiquitin, p62 and LC3 antibodies revealed fibrillar aggregates and largely excluded Pα-syn* (FIG. 4A-C). Pα-syn*, however, was found in LAMP1 positive lysosomes (FIG. 4D). These results confirmed that Pα-synF underwent autophagy, but that the autophagolysosomal digestion was incomplete resulting in Pα-syn*-bearing lysosomes. On the other hand, 20S proteasomal degradation seemed to be non-specific. In cells containing abundant Pα-synF, the 20S proteasome was found associated with Pα-synF fibrils but not with Pα-syn* aggregates (FIGS. 16A&C). In cells with low Pα-synF load, the 20S proteasome also localized with Pα-syn* (FIGS. 16B&D). In the situation of abundant Pα-synF, the 20S proteasome subunit appeared upregulated as can be seen by the more abundant staining both in cytoplasm and nucleus when compared to the PBS control or the “low fibril” situation (FIG. 16). 20S proteasome upregulation was observed starting at 8 days post PFF seeding in neurons with a high load of fibrils, and seemed to be a reaction to the failure of the cells to contain large Pα-synF fibrils. A quantitative colocalization analysis was performed. FIG. 16E shows the Manders' colocalization coefficients for Pα-synF and Pα-syn*, confirming the observations that the vast majority of Pα-synF was tagged by ubiquitin for degradation, subsequently engaging p62 and LC3. On the other hand, Pα-syn* colocalized mostly with LAMP1, and both Pα-synF and Pα-syn* colocalized with the 20S proteasome.

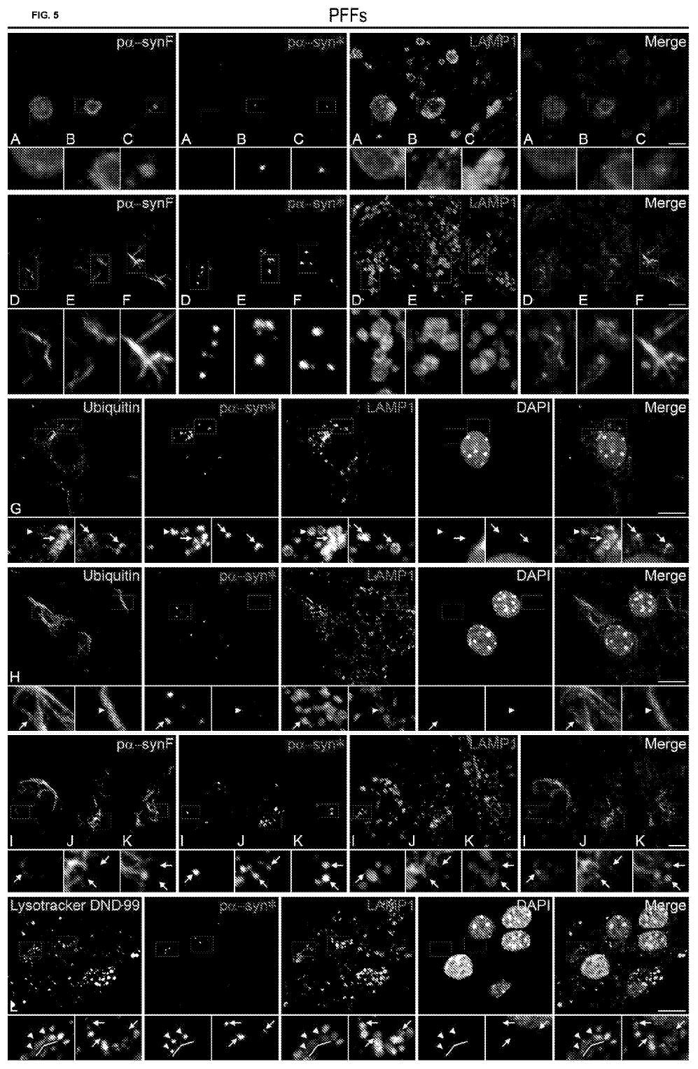

It appears that Pα-synF fibrils growth was highly dynamic and accompanied with simultaneous formation of Pα-syn*, as α-synF aggregates engulfed in LAMP1 vesicles (autophagolysosomes or lysosomes) were found at days 2-3, before fibrils were seen in the cells (FIG. 5A-C, see small Pα-syn* puncta in B and C) and when Pα-synF forms short protofibrils (FIG. 5D-F). Later on, when cells contained Pα-synF fibrils, LAMP1 vesicles were found engulfing the core of the fibrils (FIG. 5G) or surrounding the fibrils (FIG. 5H), and always containing Pα-syn* aggregates. The last step of Pα-syn* formation was the exit from the lysosomes, depicted in FIG. 5I-K (see arrows pointing to Pα-syn* aggregates exiting disrupted lysosomes).

The fluorescent dye Lysotracker Red DND-99, which stains acidic organelles, was used. In Pα-syn*-laden lysosomes (FIG. 5L, arrows in the right zoom square) Lysotracker DND-99 staining was absent, contrasting with the positive Lysotracker DND-99 signal in Pα-syn* negative, LAMP1-positive, vesicles in close vicinity. This observation proides evidence that lysosomes bearing Pα-syn* inclusions lack the acidic internal environment, probably as a consequence of Pα-syn* accumulation. Interestingly, some elongated vesicles showing weak LAMP1 staining but a good Lysotracker DND-99 signal contained Pα-syn* inclusions organized in beads-like shape, providing evidence they were autophagolysosomes degrading a fibril (FIG. 5L, arrowheads in the left zoom square pointing to Pα-syn* aggregates in weakly LAMP1 positive vesicles).

### Example 5

### Autophagy Modulation Affects the Formation of Pα-syn*

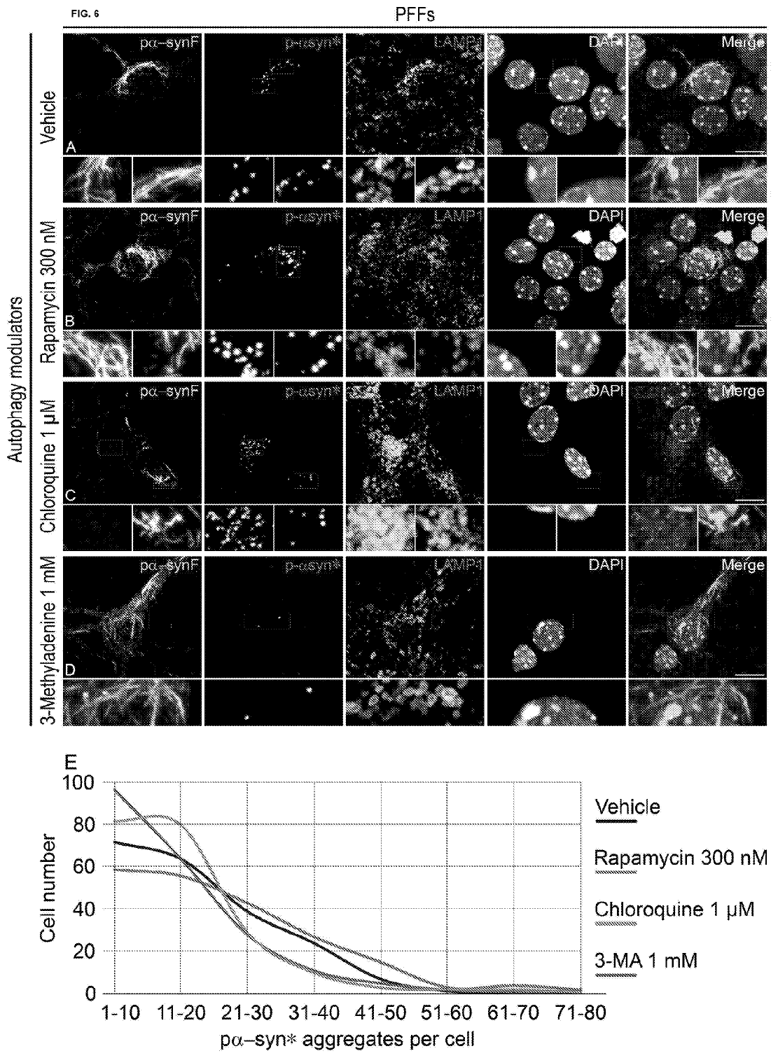

Since it was found herein, that Pα-syn* results from autophagic degradation of Pα-synF, we tested whether modulation of the autophagic flux would affect the rate of Pα-syn* formation. Neurons were treated for 2.5 days (from day 3.5 post-exposure to PFFs) with autophagy modulators and examined at the end of the treatment. Treatment with rapamycin, an autophagy activator, resulted in the formation of larger (FIG. 6B) and more numerous (FIG. 6E) Pα-syn* aggregates. Treatment with chloroquine, a compound disrupting lysosomal function, resulted in the net production of fewer Pα-syn* aggregates (FIG. 6E); additionally, in very few cells, a unique phenotype was found where cells exhibited numerous Pα-syn* aggregates in the absence of Pα-synF (FIG. 6C). This phenotype contrasts with Pα-syn* being otherwise always found in cells containing Pα-synF. It was hypothesized that chloroquine treatment, by disabling lysosomes, reduces the exit rate of Pα-syn*, slows the phagocytic flux and leads to a longer retention time of Pα-synF in autophagosomes, resulting in a more complete conversion of Pα-synF into Pα-syn*. This hypothesis also favors Pα-syn* being generated in the autophagolysosomal pathway, consistent with our observation of Pα-syn* inclusions in vesicles exhibiting features of autophagolysosomes (as described above, see FIG. 5L left zoom square). Finally, treatment with 3-Methyladenine (3-MA), a compound which impairs the formation of autophagosomes, led to a decrease in the number of Pα-syn* aggregates generated (FIGS. 6D&E).

Overall, these data showed that the cellular autophagic activity level directly affects the rate of formation of Pα-syn*.

### Example 6

### Pα-syn* Targets Mitochondria After Lysosomal Release

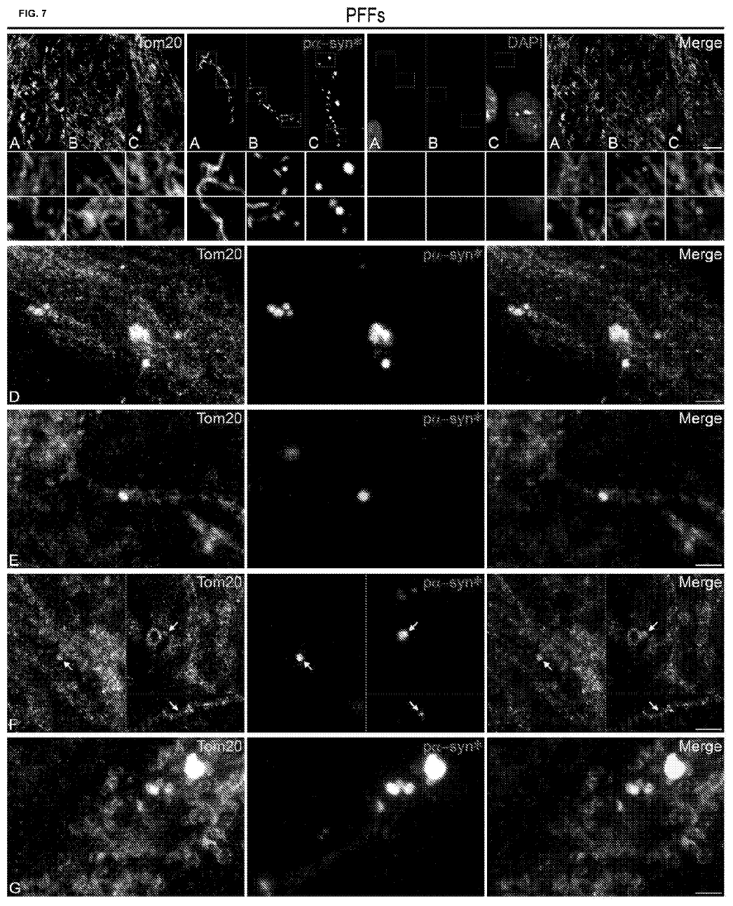

Prior to lysosomal release, nascent Pα-syn* aggregates were found in the vicinity of mitochondrial networks but not colocalizing with them, and they sometimes harbored a serpentine morphology (FIG. 7A and FIG. 3A-D). On the other hand, mature, granular Pα-syn* aggregates were found abutting the end of mitochondrial tubules (FIG. 7C). Confocal and STimulated Emission Depletion (STED) nanoscopy (with spatial resolution<50 nm) was performed to verify the direct association of Pα-syn* aggregates with the outer mitochondrial membrane (labeled with Tom20). Pα-syn* was found colocalizing with Tom20 on mitochondrial tubules (FIGS. 7D&E). FIG. 7F shows smaller Pα-syn* aggregates allowing to visualize the association with mitochondrial tubules, and also with circular mitochondrial structures that might represent fissioned mitochondria (FIG. 7F, right top panel). Pα-syn* deposition was frequently associated with areas of mitochondrial fragmentation (FIG. 7G).

### Example 7

### Pα-syn* is Mitotoxic

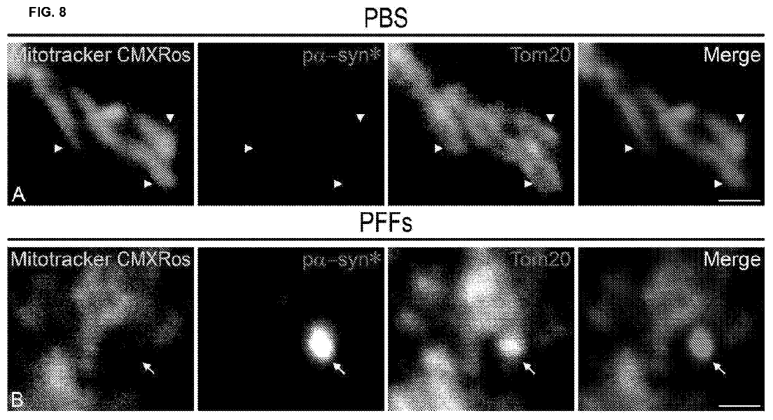

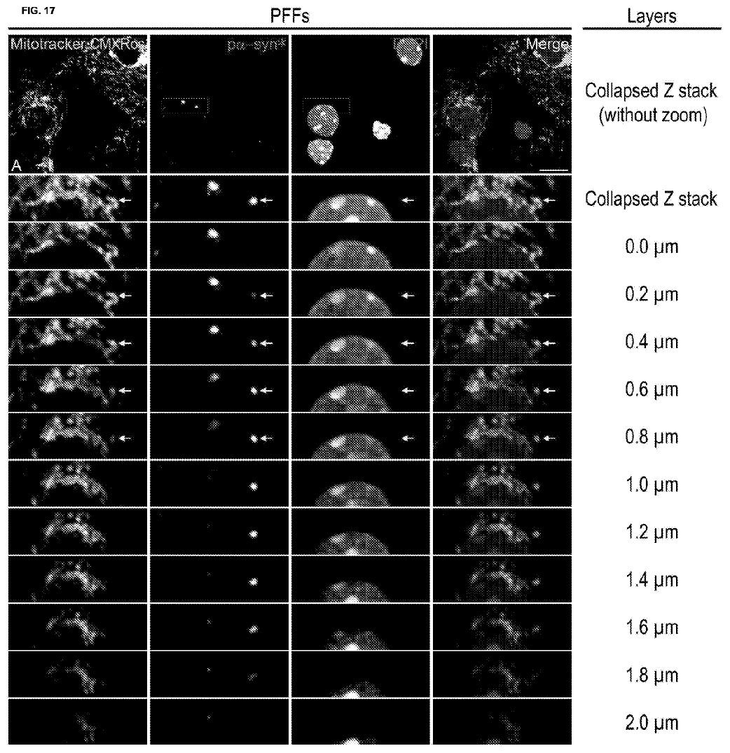

Pα-syn* was found to induce depolarization of the inner mitochondrial membrane. MitoTracker Red CMXRos is a reagent that informs on the electrochemical gradient across the inner mitochondrial membrane and thereby scores the mitochondria's ability to perform oxidative phosphorylation and produce ATP. Mitochondrial labeling with MitoTracker Red CMXRos showed that Pα-syn* was appended at the end of mitochondrial tubules that lacked membrane potential (FIG. 17). To further demonstrate this point, neurons were co-labeled for Tom20, a resident protein of the outer mitochondrial membrane that is part of the protein import machinery, and MitoTracker Red CMXRos. While the Tom20 antibody was labeling the mitochondrial extremities to which Pα-syn* was appended, MitoTracker Red CMXRos labeling was completely disrupted in such extremities (FIG. 8B, compare with FIG. 8A for co-localization of Tom20 and MitoTracker Red CMXRos in PBS-treated neurons). These data show that Pα-syn* induces a loss of membrane potential of the portion of the mitochondria to which it is attached.

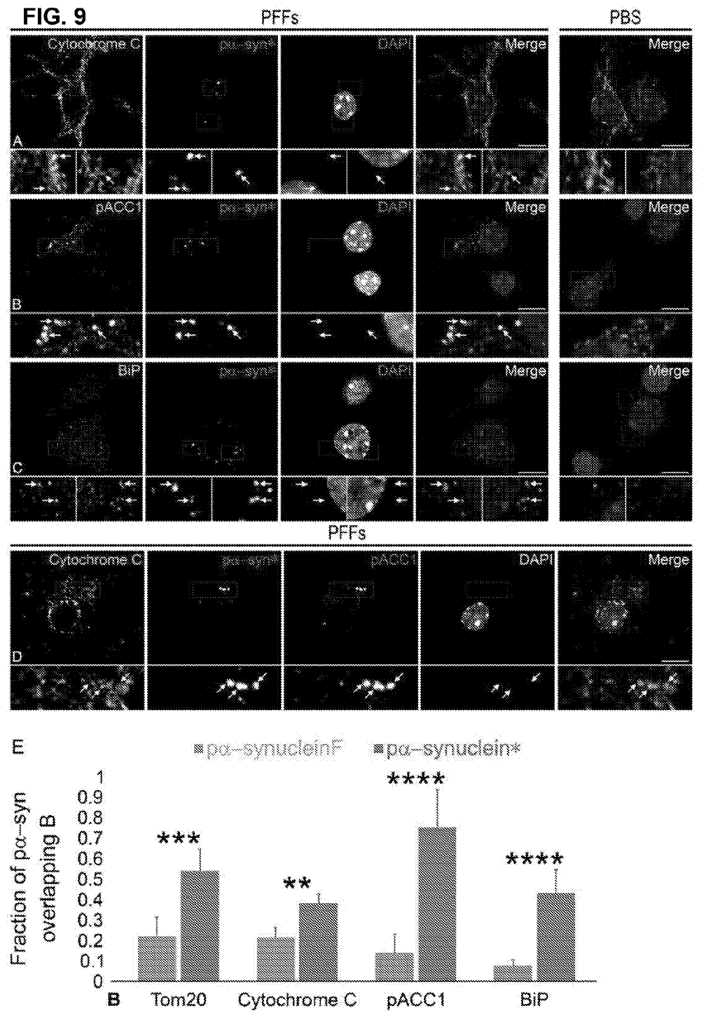

It was additionally observed that Pα-syn* induces cytochrome C release, mitochondrial fragmentation, pACC aggregate formation, and mitophagy. At the mitochondrial membrane, Pα-syn* localized usually to areas of increased cytochrome C staining (FIG. 9A, arrows). Cytochrome C accumulates in the mitochondrial intermembrane space as a result of a loss in mitochondrial potential and is then subsequently released after outer membrane permeabilization. The observation of Pα-syn* inclusions corresponding to zones of cytochrome C accumulation and release is consistent with the finding of a loss of mitochondrial potential in the areas of the mitochondrial tubules capped by Pα-syn* aggregates (see above).

A striking observation was the extensive colocalization of Pα-syn* with the phosphorylated form of acetyl-CoA carboxylase (pACC, FIGS. 9B, D and E). The filamentous pACC network was progressively lost in cells bearing Pα-syn* aggregates starting at day 6, with recruitment of punctiform pACC to the mitochondria, co-localizing with Pα-syn* (FIG. 9B, arrows). ACC is involved in lipid metabolism via the production of malonyl CoA, a substrate for the synthesis of fatty acids that are essential for mitochondrial biogenesis. ACC is inactivated by phosphorylation. It is proposed that Pα-syn* induces initial mitochondrial stress and a reduction in ATP production, resulting in high levels of AMP and activation of AMP-activated protein kinase (AMPK). Once activated, AMPK phosphorylates a number of targets, ACC being a major substrate. It is further proposed that the net increase of phosphorylated ACC at damaged mitochondria as a consequence of Pα-syn* accumulation reproduces the effect of ACC knock-down, i.e. defective lipoylation. Thus, the extensive co-localization of Pα-syn* with pACC strongly evidences that Pα-syn* interferes with energy production and fatty acids synthesis, and induces structural mitochondrial damage. Not surprisingly, Pα-syn*/pACC staining co-localized with areas of cytochrome C release (FIG. 9D).

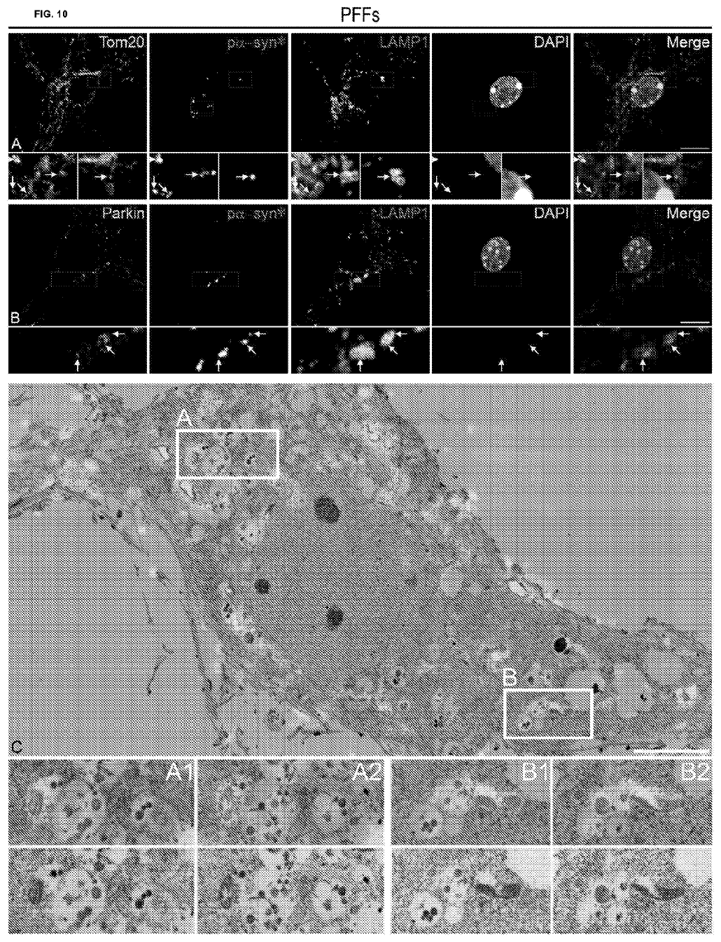

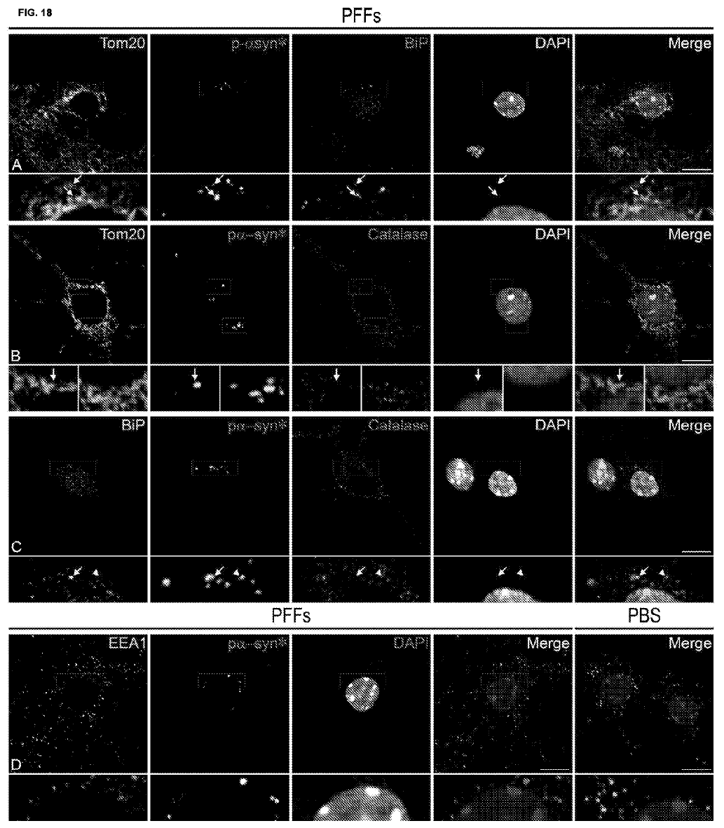

Pα-syn* colocalized with both Tom20 and BiP, a marker for the unfolded protein response and a resident protein of mitochondria associated ER membranes (MAMs), showing that Pα-syn* localized to areas of mitochondria-MAMs contact (FIG. 9C and FIG. 18A). BiP and Tom20 colocalization was enhanced in PFF-seeded cultures compared to PBS control. The quantitative co-localization analysis showed that about half of all cellular Pα-syn* is associated with Tom20 and BiP at MAMs (FIG. 9E). Disturbances to MAM-mitochondrial contacts linked to mitochondrial stress signals have been found in several neurodegenerative diseases including PD, AD and amyotrophic lateral sclerosis (ALS) and constitute an area of increased scrutiny. MAMs were found to correspond to the sites of mitochondrial fission, and autophagy induction. Therefore, the fact that Pα-syn* colocalizes with BiP at MAMs supports our data showing that Pα-syn* induces structural and functional mitochondrial damage leading to mitochondrial fission and mitophagy. Mitophagy is a parkin-dependent process, since parkin tags areas of mitochondrial damage with ubiquitin to initiate mitophagic removal. FIGS. 10A and B show the presence of Tom20/Pα-syn* bearing mitophagic lysosomes co-localizing with parkin.

In contrast to the association of Pα-syn* with the markers described above, no colocalization of Pα-syn* with the endosome marker EEA1 was found (FIG. 18). No direct association of Pα-syn* with peroxisomes was found (using the marker catalase, FIG. 18).

Electron microscopy imaging of Pα-syn* bearing mitophagic vacuoles was performed. Using Electron microscopy and Correlation Light and Electron Microscopy (CLEM) analysis, pictures of immunofluorescence (IF) labeling of Pα-syn* and cytochrome C (to localize areas of mitochondrial damage) were superimposed with ultrastructural images. Examination of cells at a late stage (14 days post-PFF exposure) revealed that virtually all Pα-syn* aggregates were contained in mitophagic vacuoles (FIG. 10C, see the vacuoles labeled in red and containing medium-density deposits corresponding to proteinaceous aggregates). These mitophagic vacuoles were in direct contact with fragmented mitochondria (harboring green labeling for cytochrome C, see insets A and B and enlarged images).

### Example 8

### Life Cycle of a Mitotoxic Pα-syn Species

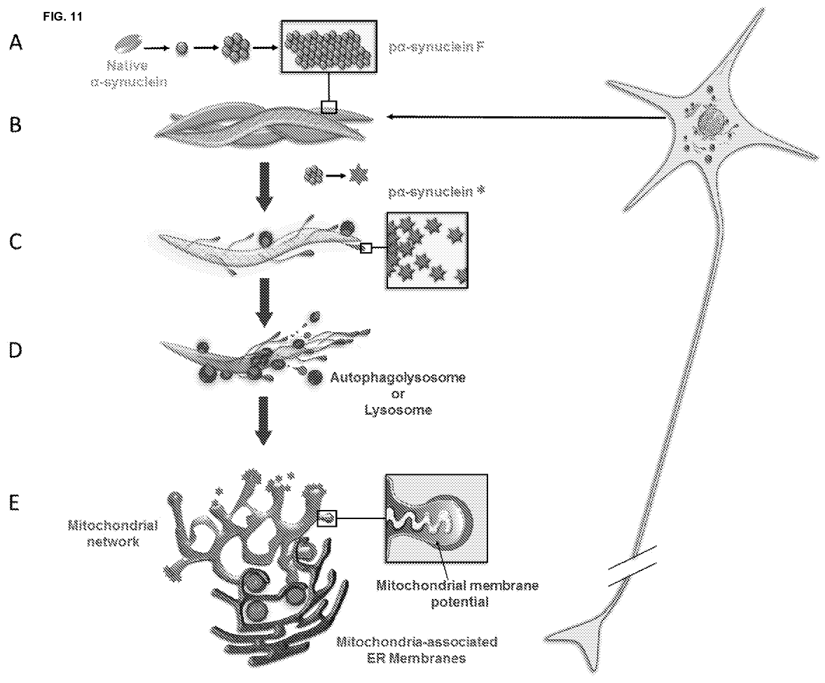

The life cycle of a mitotoxic Pα-syn species resulting from failed degradation of Pα-synF fibrils was examined. Starting from the primary observation of Pα-syn* as a unique conformational α-syn species, a series of experiments were performed to determine how Pα-syn* was generated, to which cellular organelle it localized, and what were its biological effects. The “life cycle of Pα-syn*” is depicted in FIG. 11. The model is as follows: In PFF-seeded neurons, α-syn misfolds and forms primarily fibrillar Pα-synF aggregates. Pα-synF is degraded by autophagy. However, autophagolysosomal degradation of the fibrils is incomplete and/or abnormal, generating a conformationally altered Pα-syn species, Pα-syn*, that may or may not be N- and/or C-terminally trimmed. The conformational change results directly or allosterically in exposure of a conformational epitope at the C-terminus comprising pSer129, conferring Pα-syn* novel immunoreactivity. Pα-syn* is generated in autophagolysosomes, impairs lysosomes from which it eventually exits. Pα-syn* then attaches to mitochondria where it induces functional (ACC inactivation, loss of mitochondrial potential associated with oxidative and energetic stress) and structural (mitochondrial fragmentation) damage, and cytochrome C release. Pα-syn* localizes to mitochondria-MAMs contacts, containing the misfolded protein response protein BiP (GRP78). MAMs are involved in parkin-dependent mitophagy induction, that results from Pα-syn*-induced mitochondrial fission. The life cycle of Pα-syn* ends with the mitophagic disposition of mitochondrial debris along with Pα-syn*.

### Example 9

### Pα-syn* Induces the Formation of pTau Aggregates

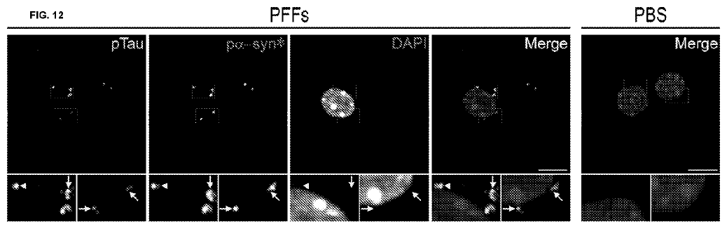

Pα-syn* colocalized with pTau aggregates as shown in FIG. 12. This provides evidence that Pα-syn* is able to trigger phosphorylation of Tau and formation of pTau aggregates at the mitochondrial membrane. These pTau aggregates are likely to add to the toxic effects of Pα-syn*.

### Example 10

### Materials and Methods

Antibody List:

Primary antibodies: Alpha-syn antibodies specific for pS129 α-syn recognizing Pα-synF, but not Pα-syn*, were mouse anti pS129 α-syn clone 81A from Biolegend (IF concentration 1/5,000, IHC concentration 1/500) and rabbit anti pS129 α-syn [MJF-R13 (8-8)] from ABCAM (used for the WB, at a concentration 1/1000). The alpha-syn antibody specific for pS129 α-syn recognizing Pα-syn*, but not Pα-synF, was rabbit anti pS129 α-syn antibody GTX50222 from GeneTex, lot 821505177 (IF concentration 1/1,000, IHC concentration 1/200, WB concentration 1/250). The data herein, indicate that this antibody, while directed against a linear epitope centered around pSer 129 of Pα-syn, recognizes a conformational epitope. Other α-syn antibodies were rabbit anti α-syn (NT) clone EP1646Y from EMD Millipore (WB concentration 1/500), rabbit anti α-syn clone D37A6 (Glu105) from Cell Signaling Technologies (WB concentration 1/1000), rabbit anti α-syn (CT) from Thermo Fisher (WB concentration 1/500). Other antibodies were rabbit anti phospho-acetyl-CoA carboxylase Ser79 from Thermo Fisher (IF concentration 1/150); rabbit anti BiP clone C50B12 from Cell Signaling Technologies (IF concentration 1/100); goat anti catalase from Novus Biological (IF concentration 1/150); mouse anti cytochrome C clone 6H2.B4 from BD Pharmingen (IF concentration 1/150); rabbit anti EEA1 clone C45B10 from Cell Signaling Technologies (IF concentration 1/100); rabbit anti GAPDH from Cell Signaling Technologies (WB concentration 1/1,000); chicken anti GFAP from Biolegend (IF concentration 1/4,000); goat anti LAMP1 from R&D Systems (IF concentration 1/400); rabbit anti LC3 from MBL (IF concentration 1/50); mouse anti NeuN clone A60 from EMD Millipore (IF concentration 1/150); mouse anti p62/SQSTM1 clone 2C11 from Abnova (IF concentration 1/150); mouse anti Parkin (PRK8) from Santa Cruz Biotechnology (IF concentration 1/50); rabbit anti Proteasome 20S core subunit from Enzo (IF concentration 1/50); mouse anti pTau (pS202-T205) clone AT8 from Thermo Fisher (IF concentration 1/200); mouse anti Tom20 clone 2F8.1 from EMD Millipore (IF concentration 1/75); rabbit anti tyrosine hydroxylase from Abcam (IHC concentration 1/750); mouse anti ubiquitin clone Ubi-1 from Thermo Fisher (IF concentration 1/750, WB concentration 1/350).

Secondary antibodies: The following secondary antibodies from Jackson ImmunoResearch Laboratories were used: ALEXA FLUOR® 488-conjugated Donkey anti Rabbit IgG (H+L), ALEXA FLUOR® 488-conjugated Donkey anti Chicken IgG (H+L), ALEXA FLUOR® 594-conjugated Donkey anti Rabbit IgG (H+L), ALEXA FLUOR® 594-conjugated Donkey anti Mouse IgG (H+L), ALEXA FLUOR® 594-conjugated Donkey anti Goat IgG (H+L), ALEXA FLUOR® 594-conjugated Donkey anti Chicken Fab2 fragment IgG (H+L), ALEXA FLUOR® 647-conjugated Donkey anti Chicken IgG (H+L). Molecular Probes (Invitrogen) antibodies were: ALEXA FLUOR® 488-conjugated anti Mouse IgG (Fab2), ALEXA FLUOR® 647-conjugated anti Mouse IgG (Fab2), ALEXA FLUOR® 647-conjugated anti Rabbit IgG (Fab2), ALEXA FLUOR® 647-conjugated Donkey anti Goat IgG (H+L). Abberior GmbH antibody was Abberior STAR RED goat anti-mouse IgG. Additionally, DAPI staining was used for nuclear counterstains.

All secondary antibodies were used for IF at a concentration of 1/1,500-1/2000, except for the Abberior STAR RED (1/100). In IHC assays, the working concentration was 1/500. For Western Blot assays, IRDye® 800CW Goat anti Rabbit IgG (H+L), IRDYE® 800CW Goat anti Mouse IgG (H+L) and IRDYE® 680LT Goat anti Mouse IgG (H+L) antibodies, all from LI-COR Biosciences, were used at a concentration of 1/2500.

Primary neuronal cultures and PFFs seeding: Primary neuronal cultures were prepared from E16-E18 C57BL/6 mouse brains (Charles River Laboratories) using standard procedures. For immunofluorescence experiments, dissociated hippocampal neurons were plated onto poly-L-lysine coated glass coverslips placed in 24-well plates, at a cell density of 125,000 cells/well. For biochemical assays, dissociated cortical neurons were plated onto poly-L-lysine-coated 6-well plates at a cell density of 1,000,000 cells/well.

During plating, the cells were maintained in DMEM plus 10% horse serum and penicillin/streptomycin for 1 hour. Thereafter, the medium was replaced and neurons were cultured in a serum free, neuron-specific, medium (NEUROBASAL® medium, N2, B27, sodium pyruvate and GLUTAMAX®, Gibco). Cultures were maintained in a humidified 37° C. incubator with 5% CO₂.

Neuronal cultures were seeded with PFFs at 5-6 days in vitro (DIV). Recombinant full length, wild-type α-syn PFFs were purified and prepared as described previously (Volpicelli-Daley et al., 2011, 2014). Briefly, α-syn PFFs were generated by incubating purified α-syn (5 mg/ml in PBS) at 37° C. with constant agitation for 5 days, followed by the preparation of aliquots and storage at −80° C. Just before seeding, PFFs were diluted in PBS at 0.1 mg/ml, sonicated during 30 sec (0.5 sec ON, 0.5 sec OFF, power 30%), and diluted in neuronal media. Two μg/ml PFFs (final) per well were added on 24-well plates for immunofluorescence experiments, and 4 μg/ml PFFs (final) per well were added to 6-well plates for biochemical assays. In the case of control conditions, an equivalent volume of PBS was added to the neuronal cultures.

Addition of α-syn monomers at a concentration of 4 μg/ml of monomer per well on 24-well plates was done as a control.

Autophagy modulation assays: Dissociated hippocampal neurons were plated onto poly-L-lysine coated glass coverslips placed in 24-well plates, at a cell density of 125,000 cells/well and seeded with PFFs (or equivalent volume of PBS for the control) at 5-6 DIV. Cells were grown in these conditions for 3.5 days (by this time, the PFF inoculum has been taken up by the neurons and/or degraded) and then autophagy modulators or the vehicle control were added: vehicle (DMSO or water), rapamycin 300 nM (SIGMA, S1039), chloroquine 1 μM (SIGMA, C6628) and 3-Methyladenine 1 mM (SIGMA, M9281). Thereafter, neuronal cultures were grown for an extra 2.5 days until a total of 6 days after PFF seeding, were fixed and processed for immunofluorescence. In order to assess the effect of autophagy modulation on Pα-syn* generation, a total of 200 cells per treatment were evaluated and the number of Pα-syn* aggregates per cells was counted in a blinded fashion at 100× magnification.

Immunofluorescence experiments: Neurons were fixed for 30 min at room temperature with 4% (w/v) paraformaldehyde in PBS containing 4% (w/v) sucrose. Neurons were washed with PBS, permeabilized with 0.2% (v/v) Triton X-100 in PBS for 6 min, washed again in PBS and blocked for 1 hour at room temperature. After labeling with a first primary antibody (overnight at 4° C.) and washing with PBS, cells were incubated with an ALEXA FLUOR® 488, 594 or 647 conjugated secondary antibody (1 hour at room temperature in the dark) and washed with PBS, stained with DAPI and mounted on microscopy slides with PROLONG® Gold antifade reagent.

In the case of double labeling experiments performed with antibodies hosted in the same species, the assays were conducted as follow. The immunofluorescence protocol was performed as described previously with the first primary and secondary antibodies. After DAPI staining a second fixation step with 4% (w/v) paraformaldehyde in PBS containing 4% (w/v) sucrose was included in order to immobilize the primary/secondary antibody complex. Cultures were washed with PBS, and blocked again for 1 hour at room temperature. The second primary antibody (generally Pα-syn81A from Covance or pS129 α-syn from GeneTex) was conjugated using Mix-n-Stain Antibody Labelling Kit (Biotium) according to the manufacturer's instructions. This conjugated antibody was then added to the cells and incubated for 1 hour at room temperature in the dark. The cells were then washed with PBS and mounted on microscopy slides with PROLONG® Gold antifade reagent.

Assays involving the use of Lysotracker Red DND-99 were performed as follow. Cells were loaded with Lysotracker Red DND-99 at a concentration of 250 μM and incubated at 37° C. for 30 min. Thereafter, the cells were washed with PBS, fixed with 4% (w/v) paraformaldehyde in PBS containing 4% (w/v) sucrose for 30 min, washed with PBS and submitted to a milder permeabilization step with 0.01% (v/v) Triton X-100 in PBS for 3 min (sufficient to allow proper antibodies staining and avoiding unloading of Lysotracker dye). Subsequent steps (blocking, labeling and mounting) were similar as described above.

Assays involving the use of Mitotracker Red CMXRos were performed as follow. Cells were loaded with Mitotracker Red CMXRos at a concentration of 250 μM and incubated at 37° C. for 30 min. Thereafter, the cells were washed with PBS and fixed with 4% (w/v) paraformaldehyde in PBS containing 4% (w/v) sucrose for 30 min. Subsequent steps (blocking, labeling and mounting) were similar as described previously. Of note, MitoTracker Red CMXRos is a red-fluorescent dye that stains mitochondria in live cells and its accumulation is dependent upon membrane potential.

Confocal and STimulated Emission Depletion (STED) nanoscopy: The cells were visualized using a spectral confocal microscope (Olympus FV1000). Images were captured and digitized using Olympus Fluoview Viewer software. In some cases, the images were analyzed using ImageJ software. All images were processed using Adobe Photoshop. Colocalization analysis was performed using the ImageJ plugin JACoP. FIGS. 1, 2, 3, 6, 13, 14 and 15 are Z-stack reconstructions (of 4-6 confocal images). Other figures are composed of confocal images (0.2 μm).

STED nanoscopy was performed on primary neurons 7 days post-treatment with PFF or PBS with a two-color Expert Line Abberior STED (Abberior Instruments GmbH, Göttingen, Germany), using the 561 nm and 640 nm pulsed excitation lasers in combination with the pulsed 775 nm STED laser. Images were recorded with a 100× oil immersion objective lens (NA 1.4), using the QUAD beam scanner. All images were recorded with both confocal resolution (i.e. with deactivated STED beam) and with 2D-STED resolution. Pixel size (x,y) was 25 nm for confocal and STED images alike, with pixel dwell times between 10-15 μs for confocal and 30-60 μs for STED images. Image stacks were typically recorded with an axial step size of 400 nm spanning a total distance between 2-4 μm. The images were recorded using line-interleaved acquisition, i.e. every line was sequentially recorded for both color channels in both confocal and STED resolution. STED laser power was typically between 40-80% of the 1250 mW STED beam (output power). The images were acquired and processed using the ImSpector software package Max-Planck Innovation), with brightness and contrast levels adjusted uniformly over the entire image stack.

Electron microscopy and Correlation Light and Electron Microscopy (CLEM) analysis: After taking fluorescence images, coverslips were gently detached from slide glasses in PBS, and cells on the coverslip were fixed with 2.5% glutaraldehyde (EM grade, 50% Aqueous Solution, Electron Microcopy Science) in 150mM cacodylate buffer (pH 7.4) at 4° C. overnight. After washing in buffer and then in water, cells were treated with 1% aqueous OsO₄ on ice for 1 hour, en-bloc stained with 1% aqueous uranyl acetate for 1 hour, dehydrated with an ethanol and acetone series, and flat-embedded in Durcupan resin (Sigma). The samples were then polymerized at 60° C. for 2 days. The entire cover glass was imaged using transmitted light with tiling (LSM 880, Carl Zeiss), and the montaged image map was used to re-find the target cell locations in the polymerized EM sample. The small region containing the target cells that were imaged with confocal microscope was trimmed and serially ultrathin sectioned at 60 nm thickness using an ATUMtome (RMC Boeckeler). The sections were collected on conductive plastic tape, aligned on a 100 mm silicon wafer, and examined in Merlin VP Compact scanning electron microscope using Atlas 5 AT software (Carl Zeiss). The area of interest was serially imaged at low-magnification and correlated back to the tiled light microscope image map. The target cell was again serially imaged with 5×5 tiling at a 30 nm/pixel resolution using the InLens Duo detector in BSE mode at 2.5 kV. The image tiles were semi-automatically stitched, and every second image was aligned using Fiji. From this stack, seven images corresponding to a representative confocal image (a thickness of 0.84 μm) were selected, and further imaged with a 15 nm/pixel resolution. One of the seven EM images was overlaid onto a high-magnification fluorescence image using Photoshop CS6. The following section was used to show the better correspondence of the fine structure to the fluorescence image in the figure.

Sequential protein extraction and western blot analyzes: Triton X-100 soluble and insoluble fractions were obtained as follow: neurons were scraped into a lysis buffer containing a mixture of basic lysis buffer (Cell Signaling Technologies), to which 1% Triton X-100, 1 mM DTT, 400 nM PMSF, and a protease and phosphatase inhibitor cocktail (Thermo Fisher) was added, at 4° C. Lysates were sonicated 10 times (0.5 sec ON, 0.5 sec OFF, power 10%), incubated 30 min and centrifuged at 25,000 g for 45 min 4° C. Thereafter, the supernatant was separated from pellet, mixed with Laemmli buffer, boiled and stored at −20° C. The pellet was washed with Triton X-100 Lysis buffer, resuspended by sonicating 10 times (0.5 sec ON, 0.5 sec OFF, power 10%), and centrifuged at 25,000 g for 45 min 4° C. The supernatant was mixed with Laemmli buffer, boiled and stored at −20° C. The pellet was resuspended in lysis buffer containing 2% SDS, sonicated 10 times (0.5 sec ON, 0.5 sec OFF, power 10%), at 4° C., mixed 3/1 with 4× Laemmli buffer, boiled and stored at −20° C.

Samples were separated and analyzed by SDS-PAGE using NUPAGE® Bis-Tris gels (10% acrylamide) and NuPAGE® MES SDS Running Buffer (Life Technologies). The resolved proteins were transferred to nitrocellulose membranes in NUPAGE® transfer buffer (Life Technologies) containing 20% methanol. The membranes were washed with Tris-buffered saline containing 0.05% Tween 20 (TTBS 0.05%) and then blocked for 1 h in BlockOut® Blocking Buffer (Rockland) at room temperature. The blots were incubated with the primary antibodies in BLOCKOUT® Buffer for 12 h at 4° C. After washing with TTBS 0.05% the membranes were incubated with Odyssey IRDye secondary antibodies (LI-COR Biosciences) for 1 hour at room temperature. After washing, the blots were imaged using an Odyssey Infrared Imaging System (LI-COR Biosciences).

For the loading controls, membranes were stripped using RESTORE™ PLUS Buffer (Thermo Fisher) for 15 min at room temperature. Thereafter, membranes were washed with TTBS 0.05% and blocked for 1 hour in BLOCKOUT® Buffer at room temperature in the dark. Then the blots were incubated with GAPDH primary antibody for 1 h at room temperature. Subsequent steps were similar as aforementioned.

Alpha-syn PFFs injections in mice: All animal experiments were performed in accordance with protocols approved by the Scripps Florida Institutional Animal Care and Use Committee (IACUC). Three months old male C57BL/6J mice (Jackson Laboratories) weighing in the range of 25 to 30 g were used. They were randomly divided into two groups to receive saline (n=6) or α-syn PFFs (n=6). Mice were acclimated for 1 week prior to initiation of study, and then anesthetized via an intraperitoneal injection of ketamine and xylazine and placed into a stereotaxic frame (Stoelting). Unilateral injections (one single dose per animal) were made into the right side of mice striatum at stereotaxic coordinates AP +0.2 mm, ML +2.0 mm and DV −2.6 mm. A volume of 2.5 μl of saline containing α-syn PFFs at a concentration of 2 μg/μl or a corresponding amount of saline alone was injected with a 26-gauge Hamilton syringe and a motorized stereotaxic injector (Stoelting) at a rate of 0.5 μl/min. The needle was left in place for 5 min following each injection before retracting to prevent backfilling along the injection tract. Formation of α-syn aggregates was allowed to proceed for 30 days. At that point, brains were collected for IHC.

Animals were euthanized by an overdose of ketamine and xylazine, followed by cardiac perfusion with 0.9% saline and then with 4% paraformaldehyde. The brains were removed and further post-fixed in 4% paraformaldehyde at 4° C. for 1 day, followed by immersion on 30% sucrose (for cryoprotection) during 3 to 4 days. Brains were embedded and frozen in optimal cutting temperature (OCT) compound and stored frozen at −80° C. until sectioning. Symmetrical 40 μm thick sections were cut on a cryostat (Leica CM3050S) from +0.2 to −4.0 mm relative to the bregma, and some sections including portions of striatum or substantia nigra (˜21-24 slices) were processed for IHC by the free floating method. Briefly, free floating brain sections were placed into PELCO PREP-EZE™ 24 well plate mesh inserts (TedPella Inc.) under constant agitation and washed several times with TTBS 0.1% to remove excess of OCT and cryoprotectant. Then, samples were pretreated with 0.3% hydrogen peroxide for 15 min, washed with TTBS 0.1% and then blocked with 4% bovine serum albumin (BSA) for 1 h at room temperature. After labeling with a first primary antibody (overnight at 4° C.) and washing with TTBS 0.1%, brain sections were incubated with an ALEXA FLUOR 488 or 594 conjugated secondary antibody (2 hours at RT in the dark) and washed again with TTBS 0.1%. Sections were mounted on Superfrost Plus slides, and a drop of Fluoroshield mounting medium with DAPI (1:4) was applied to each section. Slides were then sealed using coverslips and nail polish and stored at 4° C.

It is noteworthy to mention that, for proper identification of the substantia nigra pars compacta (SNpc), some brain sections were incubated overnight at 4° C. with antibody against tyrosine hydroxylase (TH). Those slices adjacent to TH-positive sections were then selected to staining with for pS129 α-syn specific antibodies.

Brain sections were visualized using a spectral confocal microscope (Olympus FV1000). Images were captured and digitized using Olympus Fluoview Viewer software. In some cases, the images were analyzed using ImageJ software. All images were processed using Adobe Photoshop.

Postmortem human brain tissues: Fixed brain necropsies sections of frontal cortex of elderly subjects ranging from 65 to 90 years old were kindly donated by the National Brain and Tissue Resource for Parkinson's Disease and Related Disorders, Banner Sun Health Research Institute (Sun City, Ariz.)—The Brain and Body Donation Program (BBDP). Samples received included 40 μm free floating sections fixed in 4% buffered formaldehyde from 8 patients without Parkinson's disease, 8 PD patients classified as “low Lewy Bodies”, and 8 PD patients classified as “high Lewy Bodies”. Subjects were classified according to Braak's scoring from II to V. Non Parkinson's disease subjects were used as control cases in this study.

In order to perform phospho-α-synuclein staining, a standard protocol for the neuropathological evaluation of all BBDP autopsied brains was used (with slightly modifications). Briefly, 40 μm thick free floating brain sections were sectioned in small pieces, placed into PELCO Prep-Eze™ 24-well plate mesh inserts (TedPella Inc.) under constant agitation and washed several times in PBS plus Triton X-100 0.1% in PBST 0.1% to remove the cryoprotectant. Thereafter, sections were incubated with formic acid 70% at 37° C. for 10 min (antigen retrieval), washed again with PBST 0.1% and then blocked with horse serum 10% at room temperature for 2 hours. After labeling with a first primary antibody (overnight at 4° C.) and washing with PBS, cells were incubated with an ALEXA FLUOR 488 or 594 conjugated secondary antibody (2 hours at room temperature in the dark) and washed with PBS, stained with DAPI and mounted on microscopy slides plus coverslips with ProLong Gold antifade reagent and stored at 4° C.

Brain sections were visualized using a spectral confocal microscope (Olympus FV1000). Images were captured and digitized using Olympus Fluoview Viewer software. In some cases, the images were analyzed using ImageJ software. All images were processed using Adobe Photoshop.

Statistical analyzes: The statistical significance of differences in colocalization between Pαsyn* and PαsynF was evaluated using an unpaired student's t test (GraphPad Prism v6).

### Example 11

### Pα-syn* Mitotoxicity is Linked to MAPK Activation and Involves Tau Phosphorylation and Aggregation at the Mitochondria

Herein, it is shown that Pα-syn* triggers the activation of several mitogen-activated protein kinases (MAPKs) including mitogen-activated protein kinase kinase 4 (MKK4), c-Jun N-terminal kinase (JNK), p38 and extracellular signal-regulated kinase 5 (ERK5), that are all found phosphorylated in Pα-syn* inclusions. pJNK colocalized with pα-syn* at mitochondria and mitochondria-associated ER membranes where it was associated with BiP and pACC, markers for the ER and energy deprivation, respectively. It is also shown that Pα-syn* triggers the activation of the non-MAPK glycogen synthase kinase 3 beta (GSK3β). Further, it is shown that Pα-syn* induces the formation of small ptau aggregates that are tightly associated with Pα-syn*. Pα-syn*/ptau inclusions localized to areas of mitochondrial damage and to mitophagic vesicles, showing their role in mitochondrial toxicity, mitophagy induction and their removal along with damaged mitochondrial fragments. These results add insight into the mechanisms by which Pα-syn* exerts its toxic effects that include the phosphorylation of several kinases of the MAPK pathway, as well as the formation of ptau at the mitochondrial membrane, likely contributing to mitotoxicity. Thus Pα-syn* appears to be the trigger of a series of kinase mediated pathogenic events and a link between α-syn pathology and tau, another protein known to aggregate in Parkinson's disease and other synucleinopathies.

### Material and Methods

### Antibody List

Primary antibodies: Alpha-syn antibodies specific for phospho-S129 α-syn recognizing Pα-synF, but not Pα-syn*, were mouse anti pS129 α-syn clone 81A from Biolegend (IF concentration 1/5,000, IHC concentration 1/500) and rabbit anti pS129 α-syn antibody GTX54991 from GeneTex (IF concentration 1/350). The alpha-syn antibody specific for pS129 α-syn recognizing Pα-syn*, but not Pα-synF, was rabbit anti pS129 α-syn antibody GTX50222 from GeneTex, lot 821505177 (IF concentration 1/1,000, IHC concentration 1/200, WB concentration 1/250). Other antibodies were rabbit anti phospho-acetyl-CoA carboxylase Ser79 from Thermo Fisher (IF concentration 1/150); rabbit anti BiP clone C50B12 from Cell Signaling Technologies (IF concentration 1/100); rabbit anti phospho-cJun Ser73 from Thermo Fisher (IF concentration 1/150); goat anti catalase from Novus Biological (IF concentration 1/150); mouse anti cytochrome C clone 6H2.B4 from BD Pharmingen (IF concentration 1/150); rabbit anti EEA1 clone C45B10 from Cell Signaling Technologies (IF concentration 1/100); mouse anti phospho-ERK1 Thr202/Tyr204 clone 4B11B69 from Biolegend (IF concentration 1/125); goat anti phospho-ERK5 Thr218/Tyr220 from Santa Cruz (IF concentration 1/200); chicken anti GFAP from Biolegend (IF concentration 1/4,000); goat anti LAMP1 from R&D Systems (IF concentration 1/400); rabbit anti phospho-GSK3β Ser9 from Thermo Fisher (IF concentration 1/125); rabbit anti phospho-JNK1+JNK2+JKN3 Thr183/Tyr185 from Thermo Fisher (IF concentration 1/650, IHC concentration 1/200), chicken anti phospho-JNK Thr183/Tyr185 from Thermo Fisher (IF concentration 1/500); rabbit anti phospho MKK4 Ser80 from GeneTex (IF concentration 1/100); rabbit anti phospho MKK4 Thr261 from GeneTex (IF concentration 1/150); rabbit anti phospho MKK7 Ser271/Thr275 from Bioss (IF concentration 1/150); mouse anti NeuN clone A60 from EMD Millipore (IF concentration 1/150); rabbit anti phospho-p38 Thr180/Tyr182 from Thermo Fisher (IF concentration 1/250); mouse anti parkin (PRK8) from Santa Cruz Biotechnology (IF concentration 1/50); mouse anti phospho-Tau Ser202/Thr205 clone AT8 from Thermo Fisher (IF concentration 1/250); rabbit anti phospho-Tau Ser199 clone 2H23L4 from Thermo Fisher (IF concentration 1/100); mouse anti Tom20 clone 2F8.1 from EMD Millipore (IF concentration 1/75); rabbit anti tyrosine hydroxylase from Abcam (IHC concentration 1/750).
Secondary antibodies: The following secondary antibodies from Jackson ImmunoResearch Laboratories were used: ALEXA FLUOR® 488-conjugated Donkey anti Rabbit IgG (H+L), ALEXA FLUOR® 488-conjugated Donkey anti Chicken IgG (H+L), ALEXA FLUOR® 594-conjugated Donkey anti Rabbit IgG (H+L), ALEXA FLUOR® 594-conjugated Donkey anti Mouse IgG (H+L), ALEXA FLUOR® 594-conjugated Donkey anti Goat IgG (H+L), ALEXA FLUOR® 594-conjugated Donkey anti Chicken Fab2 fragment IgG (H+L), ALEXA FLUOR® 647-conjugated Donkey anti Chicken IgG (H+L). Molecular Probes (Invitrogen) antibodies were: ALEXA FLUOR® 488-conjugated anti Mouse IgG (Fab2), ALEXA FLUOR® 647-conjugated anti Mouse IgG (Fab2), ALEXA FLUOR® 647-conjugated anti Rabbit IgG (Fab2), ALEXA FLUOR® 647-conjugated Donkey anti Goat IgG (H+L). All secondary antibodies were used for IF at a concentration of 1/1,500-1/2000.

### Primary Neuronal Cultures and PFFs Seeding

Primary neuronal cultures were prepared from E16-E18 C57BL/6 mouse brains (Charles River Laboratories) using standard procedures.

For immunofluorescence experiments, dissociated hippocampal neurons were plated onto poly-L-lysine coated glass coverslips placed in 24-well plates, at a cell density of 125,000 cells/well.

During plating, the cells were maintained in DMEM plus 10% horse serum and penicillin/streptomycin for 1 hour. Thereafter, the medium was replaced and neurons were cultured in a serum free, neuron-specific, medium (NEUROBASAL® medium, N2, B27, sodium pyruvate and GLUTAMAX®, Gibco). Cultures were maintained in a humidified 37° C. incubator with 5% CO₂.

Neuronal cultures were seeded with PFFs at 5-6 days in vitro (DIV). Recombinant full length, wild-type α-syn PFFs were purified and prepared as described previously (Volpicelli-Daley et al., 2014b; Volpicelli-Daley et al., 2011). Briefly, α-syn PFFs were generated by incubating purified α-syn (5 mg/ml in PBS) at 37° C. with constant agitation for 5 days, followed by the preparation of aliquots and storage at −80° C. Just before seeding, PFFs were diluted in PBS at 0.1 mg/ml, sonicated during 30 sec (0.5 sec ON, 0.5 sec OFF, power 30%), and diluted in neuronal media. Two μg/ml PFFs (final) per well were added on 24-well plates for immunofluorescence experiments. In the case of control conditions, an equivalent volume of PBS was added to the neuronal cultures.

Addition of α-syn monomers at a concentration of 4 μg/ml of monomer per well on 24-well plates was done as a control.

### Immunofluorescence Experiments

Neurons were fixed for 30 min at room temperature with 4% (w/v) paraformaldehyde in PBS containing 4% (w/v) sucrose. Neurons were washed with PBS, permeabilized with 0.2% (v/v) Triton X-100 in PBS for 6 min, washed again in PBS and blocked for 1 hour at room temperature. After labeling with a first primary antibody (overnight at 4° C.) and washing with PBS, cells were incubated with an ALEXA FLUOR 488, 594 or 647 conjugated secondary antibody (1 hour at room temperature in the dark) and washed with PBS, stained with DAPI and mounted on microscopy slides with ProLong Gold antifade reagent.

Assays involving the use of Mitotracker Red CMXRos were performed as follow. Cells were loaded with Mitotracker Red CMXRos at a concentration of 250 μM and incubated at 37° C. for 30 min. Thereafter, the cells were washed with PBS and fixed with 4% (w/v) paraformaldehyde in PBS containing 4% (w/v) sucrose for 30 min. Subsequent steps (blocking, labeling and mounting) were similar as described previously. Of note, MitoTracker Red CMXRos is a red-fluorescent dye that stains mitochondria in live cells and its subcellular colocalization requires the existence of a membrane potential.

### Confocal Microscopy

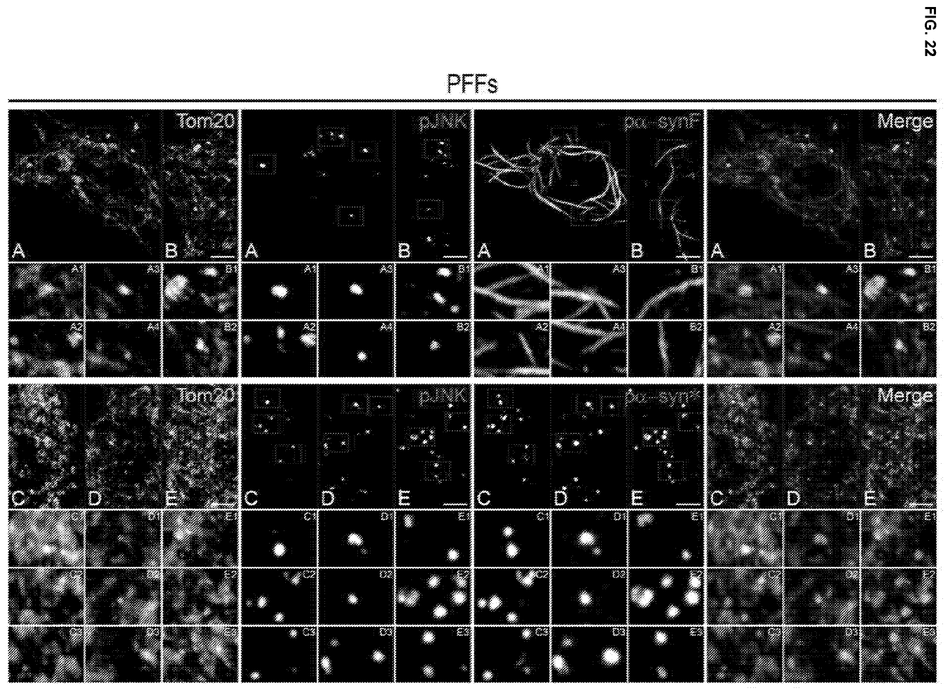

The cells were visualized using a spectral confocal microscope (Olympus FV1000). Images were captured and digitized using Olympus Fluoview Viewer software. FIG. 22 is a Z-stack reconstruction (of 4-6 confocal images). Other figures are composed of confocal images (0.2 μM). In some cases, the images were analyzed using ImageJ software. All images were processed using Adobe Photoshop.

### Quantitative Colocalization Studies

Colocalization analysis was performed using the ImageJ plugin JACoP (Bolte and Cordelieres, 2006). The Mander's colocalization coefficient (MCC) was used to measure the fraction of one protein colocalizing with another protein independently of the existence of a linear correlation between signal intensities (Dunn et al., 2011; Zinchuk and Grossenbacher-Zinchuk, 2014).

Statistical analyses were performed using the two-tailed t-test when two values were compared, and ANOVA for multiple comparisons (Prism v7).

### Anisomycin Treatment

Neurons were treated with anisomycin or the DMSO vehicle (A9789, Sigma) at a concentration of 25 μg/mL and incubated at 37° C. for 30 min. Following treatment, cells were washed with PBS and fixed with 4% (w/v) paraformaldehyde in PBS containing 4% (w/v) sucrose for 30 min. Subsequent steps (blocking, labeling and mounting) were similar as described previously.

### Alpha-syn PFFs Injections in Mice

All animal experiments were performed in accordance with protocols approved by the Scripps Florida Institutional Animal Care and Use Committee (IACUC). Three months old male C57BL/6J mice (Jackson Laboratories) weighing in the range of 25 to 30 g were used. They were randomly divided into two groups to receive saline (n=6) or α-syn PFFs (n=6). Mice were acclimated for 1 week prior to initiation of study, and then anesthetized via an intraperitoneal injection of ketamine and xylazine and placed into a stereotaxic frame (Stoelting). Unilateral injections (one single dose per animal) were made into the right side of mice striatum at stereotaxic coordinates AP +0.2 mm, ML +2.0 mm and DV −2.6 mm. A volume of 2.5 μl of saline containing α-syn PFFs at a concentration of 2 μg/μl or a corresponding amount of saline alone was injected with a 26-gauge Hamilton syringe and a motorized stereotaxic injector (Stoelting) at a rate of 0.5 μl/min. The needle was left in place for 5 min following each injection before retracting to prevent backfilling along the injection tract. Formation of α-syn aggregates was allowed to proceed for 30 days. At that point, brains were collected for IHC.

Animals were euthanized by an overdose of ketamine and xylazine, followed by cardiac perfusion with 0.9% saline and then with 4% paraformaldehyde. The brains were removed and further post-fixed in 4% paraformaldehyde at 4° C. for 1 day, followed by immersion on 30% sucrose (for cryoprotection) during 3 to 4 days. Brains were embedded and frozen in optimal cutting temperature (OCT) compound and stored frozen at −80° C. until sectioning. Symmetrical 40 μm thick sections were cut on a cryostat (Leica CM3050S) from +0.2 to −4.0 mm relative to the bregma, and some sections including portions of striatum or substantia nigra (˜21-24 slices) were processed for IHC by the free-floating method. Briefly, free floating brain sections were placed into PELCO PREP-EZE™ 24 well plate mesh inserts (TedPella Inc.) under constant agitation and washed several times with TTBS 0.1% to remove excess of OCT and cryoprotectant. Then, samples were pretreated with 0.3% hydrogen peroxide for 15 min, washed with TTBS 0.1% and then blocked with 4% bovine serum albumin (BSA) for 1 h at room temperature. After labeling with a first primary antibody (overnight at 4° C.) and washing with TTBS 0.1%, brain sections were incubated with an ALEXA FLUOR 488 or 594 conjugated secondary antibody (2 hours at RT in the dark) and washed again with TTBS 0.1%. Sections were mounted on Superfrost Plus slides, and a drop of Fluoroshield mounting medium with DAPI (1:4) was applied to each section. Slides were then sealed using coverslips and nail polish and stored at 4° C.

It is noteworthy to mention that, for proper identification of the substantia nigra pars compacta (SNpc), some brain sections were incubated overnight at 4° C. with antibody against tyrosine hydroxylase (TH). Those slices adjacent to TH-positive sections were then selected to staining with for pS129 α-syn specific antibodies.

Brain sections were visualized using a spectral confocal microscope (Olympus FV1000). Images were captured and digitized using Olympus Fluoview Viewer software. In some cases, the images were analyzed using ImageJ software. All images were processed using Adobe Photoshop.

### Postmortem Human Brain Tissues

Fixed brain necropsies sections of frontal cortex of elderly subjects ranging from 65 to 90 years old were kindly donated by the National Brain and Tissue Resource for Parkinson's Disease and Related Disorders, Banner Sun Health Research Institute (Sun City, Ariz.)—The Brain and Body Donation Program (BBDP). Samples received included 40 iim free floating sections fixed in 4% buffered formaldehyde from 8 patients without Parkinson's disease, 8 PD patients classified as “low Lewy Bodies”, and 8 PD patients classified as “high Lewy Bodies”. Subjects were classified according to Braak's scoring from II to V. Non Parkinson's disease subjects were used as control cases in this study.

A standard protocol for the neuropathological evaluation of all BBDP autopsied brains was used (with slight modifications). Briefly, 40 μm thick free floating brain sections were sectioned in small pieces, placed into PELCO PREP-EZE™ 24-well plate mesh inserts (TedPella Inc.) under constant agitation and washed several times in PBS plus Triton X-100 0.1% in PBST 0.1% to remove the cryoprotectant. Thereafter, sections were incubated with formic acid 70% at 37° C. for 10 min (antigen retrieval), washed again with PBST 0.1% and then blocked with horse serum 10% at room temperature for 2 hours. After labeling with a first primary antibody (overnight at 4° C.) and washing with PBS, sections were incubated with an ALEXA FLUOR 488 or 594 conjugated secondary antibody (2 hours at room temperature in the dark) and washed with PBS, stained with DAPI and mounted on microscopy slides plus coverslips with ProLong Gold antifade reagent and stored at 4° C.

Brain sections were visualized using a spectral confocal microscope (Olympus FV1000). Images were captured and digitized using Olympus Fluoview Viewer software. In some cases, the images were analyzed using ImageJ software. All images were processed using Adobe Photoshop.

### Results

Early activation of JNK in Pα-syn* inclusions. The objective was to determine which molecular pathway(s) are involved in the mitochondrial toxicity induced by Pα-syn*. Since JNK phosphorylation has been shown in the brains of PD patients and in PD mouse models (Ferrer et al., 2001; Hunot et al., 2004) PFF-exposed primary mouse neurons were labeled with an antibody against pJNK. pJNK labeling appeared as small inclusions present as early as 2 days after seeding, the number of which progressively increased in the culture in a manner highly reminiscent of Pα-syn* propagation (see earlier description herein). No pJNK labeling was detected after exposure of primary neurons to monomeric α-syn. pJNK labeling was specific to neuronal cells. Of note, by “pJNK labeling”, describes pJNK labeling found exclusively in PFF-treated neurons in experimental conditions where physiological axonal pJNK was not detected, i.e. the concentration of pJNK antibodies used was appropriate for the detection of somatic pJNK aggregates without significant staining of the physiological levels of axonal pJNK.

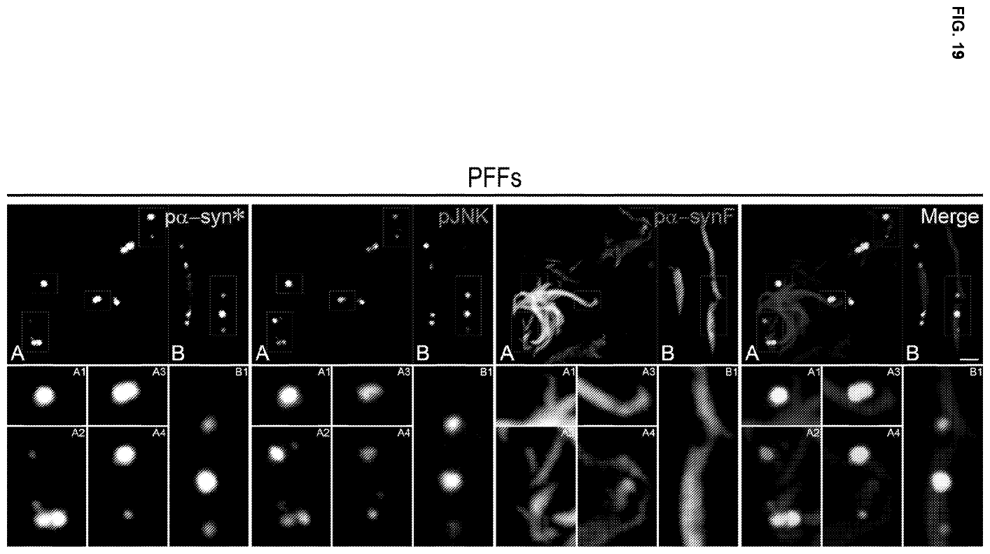

JNK activation is specific for Pα-syn* over Pα-synF. pJNK inclusions tightly co-localized with small Pα-syn* aggregates, but not Pα-synF fibrils (FIG. 19). Pα-syn* and Pα-synF are recognized by two different α-syn antibodies. Pα-syn*/JNK labeling and Pα-synF labeling were mutually exclusive. As described previously herein, Pα-syn* inclusions were released from Pα-synF, with pJNK being present in Pα-syn* positive inclusions as early as these were observable. FIGS. 19A1 & 19B1 show several indents in Pα-synF fibers, with the presence of newly formed pJNK positive Pα-syn* aggregates.

JNK is also activated in brains of PFF-injected mice and PD patients. pJNK positive inclusions were observed in the brains of mice stereotaxically injected with PFFs and in PD patients brains, confirming the biological relevance of the findings in primary neuronal cultures.

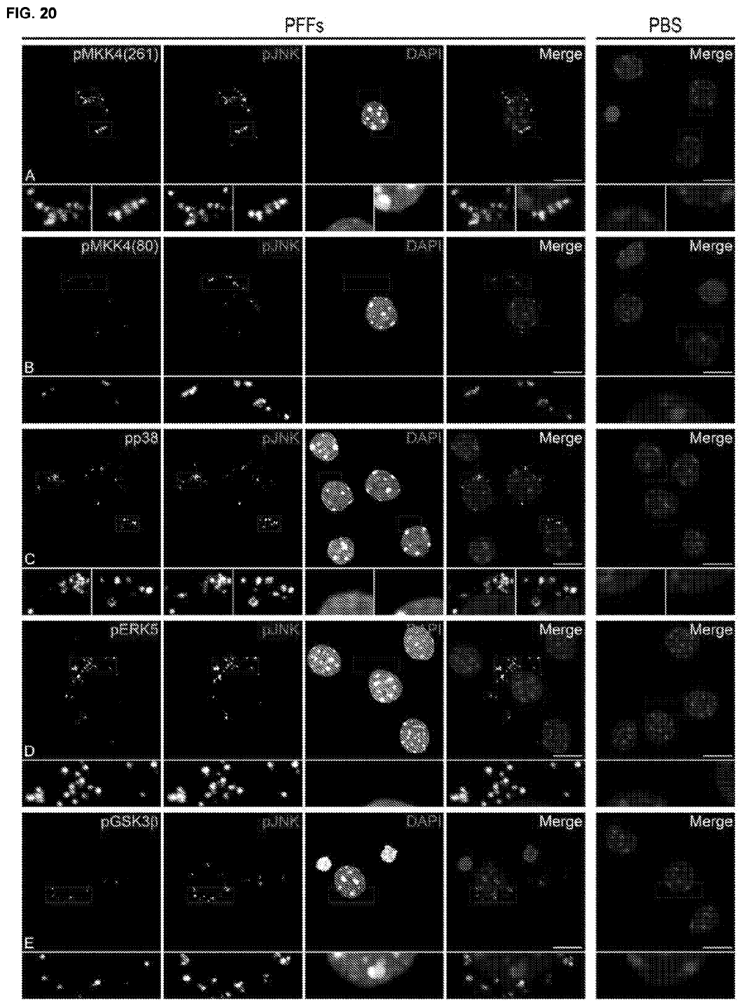

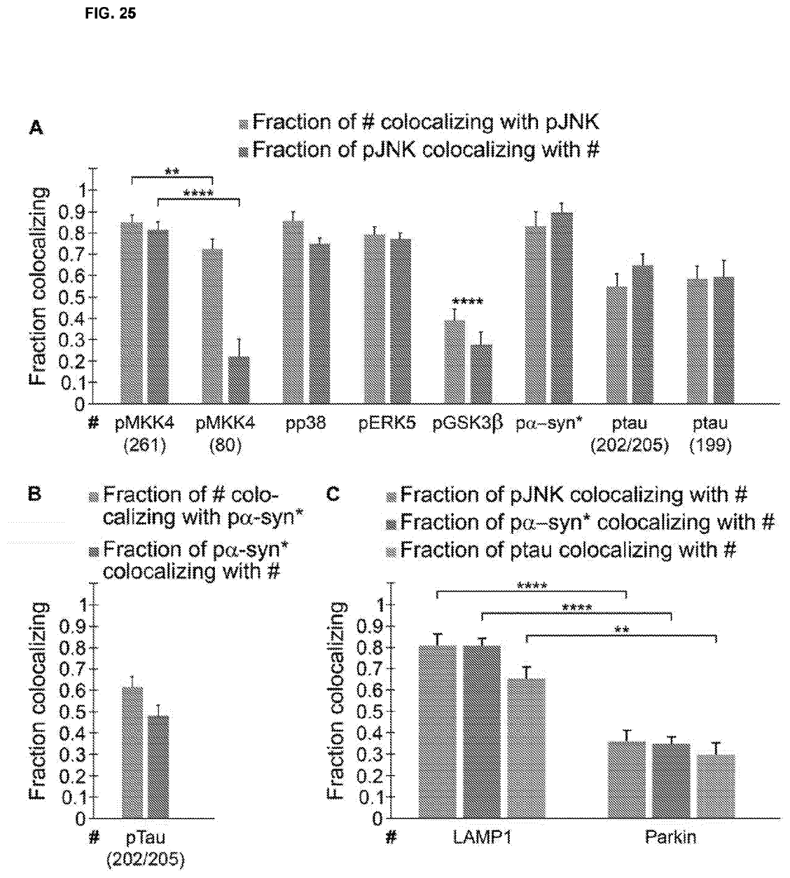

Pα-syn* inclusions triggers phosphorylation of several members of the MAPK family of kinases. pJNK activation was not related to canonical Jun phosphorylation. It was further observed that other members of the MAPK family of kinases were phosphorylated in Pα-syn* inclusions (FIGS. 20A-20E and 25A-25C). The activated form of MKK4, a kinase that activates JNK and p38 (Cuenda, 2000), exhibited tight colocalization with pJNK. Abundant T261-phosphorylated (activated) MKK4 was present in Pα-syn*/pJNK inclusions (FIG. 20A), contrasting with very rare S80-phosphorylated (inactivated) (Crittenden and Filipov, 2011) MKK4 (FIG. 20B), indicating that Pα-syn* was associated with the activated, but not the inactivated form of the enzyme. Phosphorylated p38 was also co-localizing with Pα-syn*/pJNK inclusions, as well as pERK5 (FIGS. 20C-20D). On the contrary, pMKK7 and pERK1/2 did not colocalize with Pα-syn*/pJNK inclusions, and pGSK3β, a kinase not belonging to the MAPK pathway, exhibited partial colocalization (FIG. 20E).

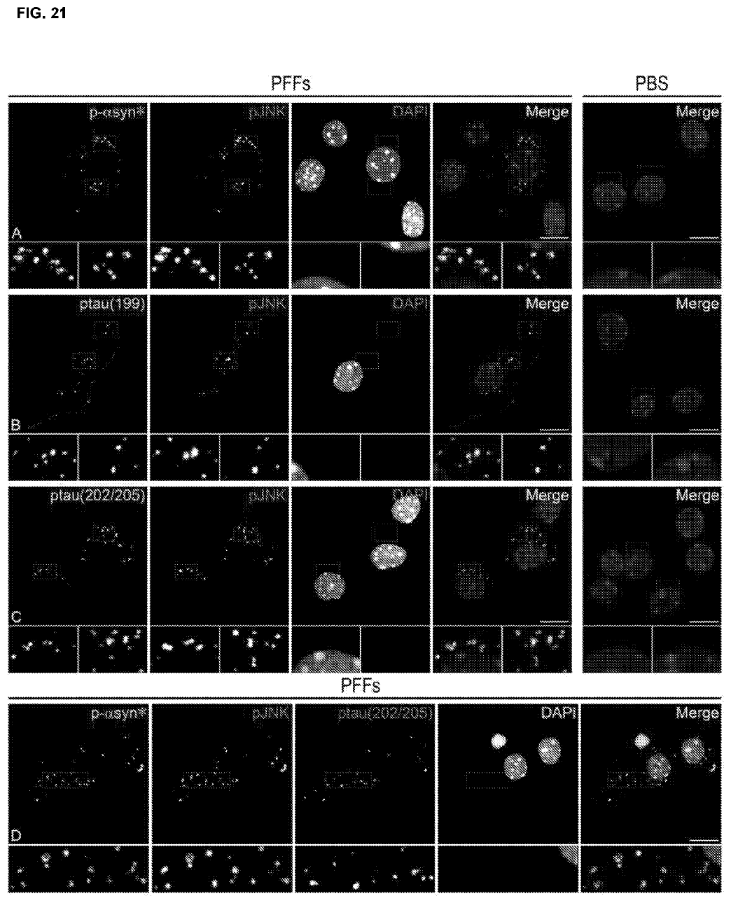

Pα-syn* inclusions are associated with ptau aggregates. Oligomeric ptau aggregates have been described at the mitochondria, and it has been suggested that these represent a toxic form of tau (Lasagna-Reeves et al., 2011). Moreover, tau pathology is found in PD patients and animal synucleinopathy models (Haggerty et al., 2011; Irwin et al., 2013; Sengupta et al., 2015), and tau is a substrate for phosphorylation by several MAPK proteins (Martin et al., 2013). It was investigated if Pα-syn*, via the tightly associated activated kinases identified above, might be the culprit in triggering tau phosphorylation, by co-immunolabeling for pJNK and/or Pα-syn*, and ptau. While Pα-syn* and pJNK were completely colocalizing (FIG. 21A), Pα-syn* and ptau aggregates were either largely overlapping (FIGS. 21B&D) or juxtaposed (FIGS. 21C&D). These observations provide evidence that Pα-syn* triggers MAPK phosphorylation, and that activated MAPK then induce tau phosphorylation and aggregation in the vicinity of Pα-syn*.

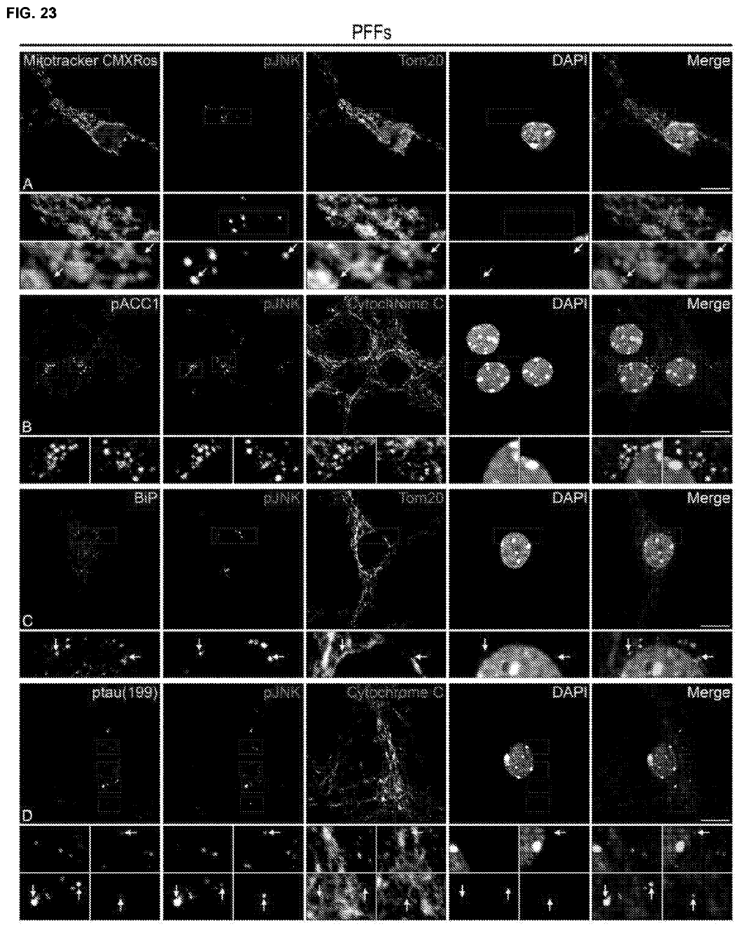

Pα-syn*/ptau aggregates colocalize at damaged mitochondria. In FIG. 19, it was demonstrated that JNK was activated in early Pα-syn* aggregates shed from Pα-synF fibrils. In FIG. 22A-E, it is shown that pJNK was still associated with mature Pα-syn* aggregates localized to the mitochondria. pJNK and Pα-syn* colocalized with Tom20, a marker of the outer mitochondrial membrane, but not with Pα-synF. FIG. 22C-E depicts areas of abundant Pα-syn*/pJNK inclusions, and fragmented mitochondria. Similar to what was described for Pα-syn* earlier herein, pJNK labeling colocalized exquisitely with areas of mitochondrial damage as shown in FIG. 23 by the following 1) loss of membrane potential (FIG. 23A), 2) pACC1 sequestration (FIG. 23B), 3) cytochrome C staining (FIG. 23C). Colocalization with BiP, a resident protein of mitochondria associated ER membranes (MAMs), indicates that Pα-syn*/pJNK inclusions occur at MAMs (FIG. 23C). Mitotracker CMXRos labeling, a marker of mitochondrial potential, was missing in the direct vicinity of pJNK punctae (FIG. 23A). Pα-syn* and ptau were both found surrounded by abundant cytochrome C staining at the mitochondria (FIG. 23D), supporting that ptau contributes to mitochondrial toxicity in synucleinopathies. A small proportion of pJNK positive aggregates were found juxtaposed to catalase positive peroxisomes. No colocalization was found with EEA1 positive early endosomes.

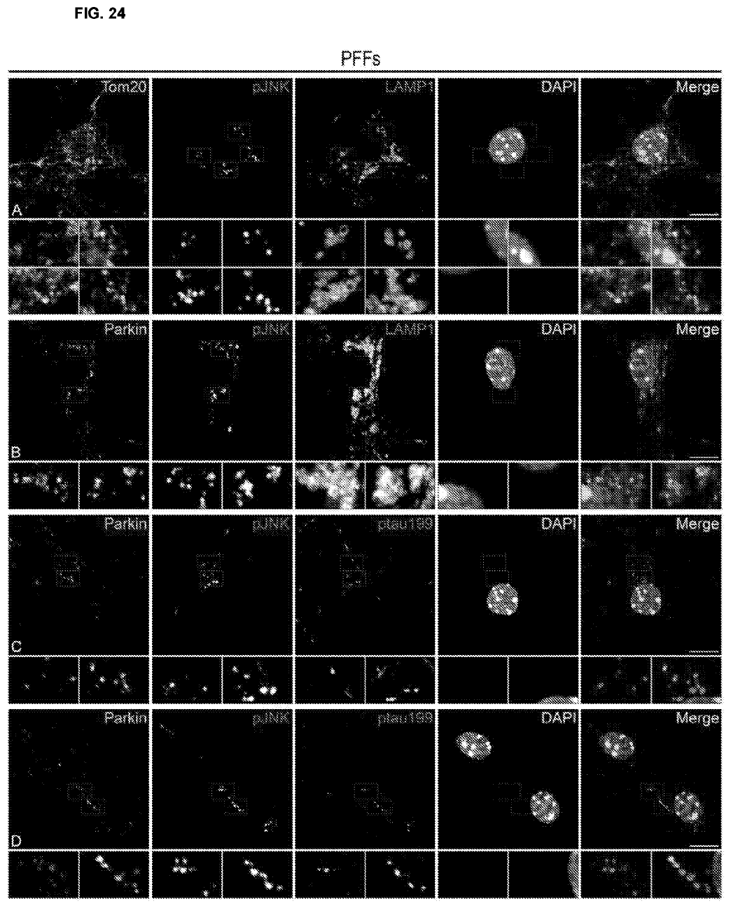

Pα-syn*/ptau aggregates are associated with mitophagy. The inventors previously showed that Pα-syn* induced mitochondrial fragmentation and mitophagy. Here, it was observed that pJNK positive inclusions colocalized with Tom20 in parkin-positive LAMP1 vesicles (FIGS. 24A-24B, FIG. 24A also shows fragmented mitochondria), showing that they underwent mitophagy. pTau colocalized with pJNK in mitophagic vesicles (FIGS. 24C-24D).

Quantitative colocalization. Ninety percent of Pα-syn* staining co-localized with pJNK staining, and vice-versa (FIG. 25A). Importantly, nearly all Pα-syn* inclusions (and all Pα-syn* positive neurons) were positive for pJNK. At times, pJNK staining in a given inclusion was more intense than Pα-syn* staining, or inversely Pα-syn* was more intensely stained than pJNK (shown in FIG. 19). Colocalization reached comparable levels with activated pMKK4, pp38 and pERK5, but not pGSK3β (FIG. 25A).

pJNK positive inclusions did trigger extremely few phosphorylation events of MKK4 at the inhibitory site S80, hence the poor colocalization of pJNK with pMKK4 (S80) (red bars); however, the few positive pMKK4 (S80) dots were colocalizing with pJNK (green bars). The difference in colocalization of pJNK with either pMKK4 (T261) or pMKK4 (S80) was highly significant statistically.

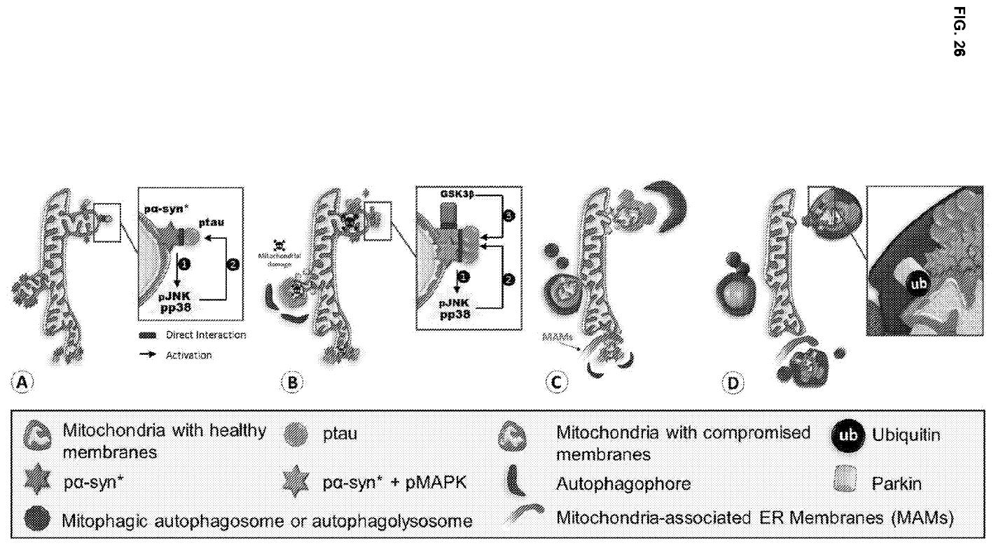

Finally, about 60% colocalization was found between Pα-syn*/pJNK positive aggregates and ptau, consistent with the observation of both protein aggregates being juxtaposed or colocalizing (FIGS. 25A and 25B). Occasionally, Pα-syn* inclusions were found without ptau. However, ptau was always seen colocalizing with Pα-syn* aggregates. The interpretation for these findings is that Pα-syn* activates kinases that then phosphorylate tau. The phosphorylation cascade as well as the recruitment of tau by Pα-syn* are not yet established in early Pα-syn* aggregates, hence the presence of some Pα-syn* aggregates in the absence of ptau. This cascade of events is described in FIGS. 26A-26D.

FIG. 25C shows that 80% of Pα-syn* and pJNK co-localize with LAMP1, in accordance with the fact that Pα-syn* is found abundantly in mitophagic vesicles. Close to 70% of ptau colocalized with LAMP1. Parkin did not associate directly with Pα-syn* inclusions (30% colocalization with Pα-syn*, ptau or pJNK). A lower colocalization of protein aggregates with parkin is to be expected since parkin ubiquinates outer mitochondrial membrane proteins to trigger selective autophagy as a response to mitochondrial damage and PINK1 accumulation at the outer membrane (Pickrell and Youle, 2015).

### Discussion

Pα-syn* is a conformationally distinct, small aggregate of Pα-syn, resulting from incomplete autophagic degradation of Lewy body type Pα-syn fibrils (Pα-synF). After exiting autolysosomes, Pα-syn* attaches to mitochondrial tubules, inducing metabolic stress, membrane depolarization and mitochondrial fragmentation. Pα-syn* is finally localized in mitophagic vacuoles surrounded by mitochondrial debris (Grassi et al., 2018).

In this study, some key molecular actors were defined in the toxic pathway elicited by Pα-syn*. Strikingly, 80-90% colocalization of Pα-syn* was observed with phosphorylated JNK (pJNK, FIGS. 19, 21A-21D and 25A-25C). pJNK colocalized with Pα-syn* early, i.e. as soon as Pα-syn* was released from Pα-synF, and was completely excluded from Pα-synF (FIG. 19). Therefore, in some experiments, pJNK was used as a surrogate marker for Pα-syn* aggregates. The Pα-syn*/pJNK aggregates also colocalized with phosphorylated MKK4, a MAP kinase kinase (MAPKK) phosphorylating and activating JNK and p38 (Cuenda, 2000) (FIGS. 20A-20E and 25A-25C). Pα-syn* led to MKK4 phosphorylation overwhelmingly at its activation site T261 (as opposed to S80 leading to the inactivation of the kinase). Pα-syn*/pJNK aggregates were also found colocalizing with pp38. On the contrary, MKK7, the second JNK activating MAPKK, was not found to be phosphorylated in the vicinity of Pα-syn*. Altogether, these data strongly evidence molecule- and site-specific activation of MKK4 by Pα-syn*. The data herein provide evidence that activation of the MAPK pathway is directly involved in the mitotoxic effects of Pα-syn*.

In this study, ptau aggregates were found directly juxtaposed and/or overlapping with Pα-syn* aggregates (FIGS. 21A-21D). The Pα-syn*/ptau aggregates were located at the mitochondrial membrane, specifically in areas of mitochondrial damage (shown by exquisite loss of mitotracker CMXRos labeling in the vicinity of Pα-syn*; clustering of pACC1 that is a marker of mitochondrial membranes structural damage; co-localization with BiP, a resident protein of MAMs that are recruited to initiate mitophagy, see FIGS. 23A-23D). A large proportion of Pα-syn*/ptau aggregates were found in LAMP-1 mitophagic vacuoles (FIGS. 24A-24D and 25A-25C). These data provide evidence that Pα-syn* and ptau act in concert to induce mitochondrial dysfunction. Emphasizing the cooperation of Pα-syn* and ptau in mitochondrial toxicity, fragmentation and mitophagy induction, it was shown that Pα-syn*/pJNK/ptau inclusions are colocalizing with Tom20 (marker of the mitochondrial membrane) in parkin decorated, LAMP1 positive mitophagic lysosomes (FIGS. 24A-24D and 25A-25C).

How does tau get phosphorylated? The data herein are interpreted as follows (FIGS. 26A-265D). Pα-syn* activates MKK4, leading to JNK and p38 activation, with both phosphorylated enzymes faithfully colocalizing with Pα-syn*. Pα-syn* directly interacts with tau, which gets phosphorylated by pJNK and pp38 (FIG. 26A). Pα-syn* and ptau aggregate into larger inclusions surrounding the endings of mitochondrial tubules. Concomitantly, GSK3β, which also directly interacts with Pα-syn*, contributes to further tau phosphorylation. Pα-syn*/ptau aggregates are toxic, leading to mitochondrial dysfunction and fragmentation (FIG. 26B). Fragmented mitochondria surrounded by Pα-syn*/ptau aggregates is tagged for mitophagic degradation by parkin (FIG. 26C). Mitophagic vacuoles are released, containing Pα-syn*/ptau, pJNK, pp38, parkin and mitochondrial debris (FIG. 26D).

It is demonstrated herein that Pα-syn*/ptau aggregates and MAPK activation are directly linked with mitochondrial damage and mitophagy. Without wishing to be bound by theory, it is hypothesized that Pα-syn* acts as the master trigger of both kinase activation and the formation of mitochondrial ptau aggregates, emphasizing the central role of Pα-syn* in the pathogenesis of Parkinson's disease and other synucleinopathies.

### REFERENCES

Alonso, A. D., J. Di Clerico, B. Li, C. P. Corbo, M. E. Alaniz, I. Grundke-Iqbal, and K. Iqbal. 2010. Phosphorylation of tau at Thr212, Thr231, and Ser262 combined causes neurodegeneration. J Biol Chem 285:30851-30860.
Arima, K., K. Ueda, N. Sunohara, K. Arakawa, S. Hirai, M. Nakamura, H. Tonozuka-Uehara, and M. Kawai. 1998. NACP/alpha-synuclein immunoreactivity in fibrillary components of neuronal and oligodendroglial cytoplasmic inclusions in the pontine nuclei in multiple system atrophy. Acta Neuropathol 96:439-444.
Athanassiadou, A., G. Voutsinas, L. Psiouri, E. Leroy, M. H. Polymeropoulos, A. Ilias, G. M. Maniatis, and T. Papapetropoulos. 1999. Genetic analysis of families with Parkinson disease that carry the Ala53Thr mutation in the gene encoding alpha-synuclein. Am J Hum Genet 65:555-558.
Betarbet, R., R. M. Canet-Aviles, T. B. Sherer, P. G. Mastroberardino, C. McLendon, J. H. Kim, S. Lund, H. M. Na, G. Taylor, N. F. Bence, R. Kopito, B. B. Seo, T. Yagi, A. Yagi, G. Klinefelter, M. R. Cookson, and J. T. Greenamyre. 2006. Intersecting pathways to neurodegeneration in Parkinson's disease: effects of the pesticide rotenone on DJ-1, alpha-synuclein, and the ubiquitin-proteasome system. Neurobiol Dis 22:404-420.
Bolte, S., and F. P. Cordelieres. 2006. A guided tour into subcellular colocalization analysis in light microscopy. J Microsc 224:213-232.
Chambers, J. W., A. Pachori, S. Howard, M. Ganno, D. Hansen, Jr., T. Kamenecka, X. Song, D. Duckett, W. Chen, Y. Y. Ling, L. Cherry, M. D. Cameron, L. Lin, C. H. Ruiz, and P. Lograsso. 2011. Small Molecule c-jun-N-terminal Kinase (JNK) Inhibitors Protect Dopaminergic Neurons in a Model of Parkinson's Disease. ACS Chem Neurosci 2:198-206.
Chang, D., M. A. Nalls, I. B. Hallgrimsdottir, J. Hunkapiller, M. van der Brug, F. Cai, C. International Parkinson's Disease Genomics, T. andMe Research, G. A. Kerchner, G. Ayalon, B. Bingol, M. Sheng, D. Hinds, T. W. Behrens, A. B. Singleton, T. R. Bhangale, and R. R. Graham. 2017. A meta-analysis of genome-wide association studies identifies 17 new Parkinson's disease risk loci. Nat Genet 49:1511-1516.
Chang, L., Y. Jones, M. H. Ellisman, L. S. Goldstein, and M. Karin. 2003. JNK1 is required for maintenance of neuronal microtubules and controls phosphorylation of microtubule-associated proteins. Dev Cell 4:521-533.
Charni, S., G. de Bettignies, M. G. Rathore, J. I. Aguilo, P. J. van den Elsen, D. Haouzi, R. A. Hipskind, J. A. Enriquez, M. Sanchez-Beato, J. Pardo, A. Anel, and M. Villalba. 2010. Oxidative phosphorylation induces de novo expression of the MHC class I in tumor cells through the ERK5 pathway. J Immunol 185:3498-3503.
Chen, J., Y. Ren, C. Gui, M. Zhao, X. Wu, K. Mao, W. Li, and F. Zou. 2018. Phosphorylation of Parkin at serine 131 by p38 MAPK promotes mitochondrial dysfunction and neuronal death in mutant A53T alpha-synuclein model of Parkinson's disease. Cell Death Dis 9:700.
Cho, J. H., and G. V. Johnson. 2004. Primed phosphorylation of tau at Thr231 by glycogen synthase kinase 3beta (GSK3beta) plays a critical role in regulating tau's ability to bind and stabilize microtubules. J Neurochem 88:349-358.
Choi, W. S., G. Abel, H. Klintworth, R. A. Flavell, and Z. Xia. 2010. JNK3 mediates paraquat- and rotenone-induced dopaminergic neuron death. J Neuropathol Exp Neurol 69:511-520.
Crittenden, P. L., and N. M. Filipov. 2011. Manganese modulation of MAPK pathways: effects on upstream mitogen activated protein kinase kinases and mitogen activated kinase phosphatase-1 in microglial cells. J Appl Toxicol 31:1-10.
Cuenda, A. 2000. Mitogen-activated protein kinase kinase 4 (MKK4). Int J Biochem Cell Biol 32:581-587.
Dawson, T. M., and V. L. Dawson. 2003. Rare genetic mutations shed light on the pathogenesis of Parkinson disease. J Clin Invest 111:145-151.
Di Maio, R., P. J. Barrett, E. K. Hoffman, C. W. Barrett, A. Zharikov, A. Borah, X. Hu, J. McCoy, C. T. Chu, E. A. Burton, T. G. Hastings, and J. T. Greenamyre. 2016. alpha-Synuclein binds to TOM20 and inhibits mitochondrial protein import in Parkinson's disease. Sci Transl Med 8:342ra378.
Duka, V., J. H. Lee, J. Credle, J. Wills, A. Oaks, C. Smolinsky, K. Shah, D. C. Mash, E. Masliah, and A. Sidhu. 2013. Identification of the sites of tau hyperphosphorylation and activation of tau kinases in synucleinopathies and Alzheimer's diseases. PLoS One 8:e75025.
Dunn, K. W., M. M. Kamocka, and J. H. McDonald. 2011. A practical guide to evaluating colocalization in biological microscopy. Am J Physiol Cell Physiol 300:C723-742.
Eriksen, J. L., S. Przedborski, and L. Petrucelli. 2005. Gene dosage and pathogenesis of Parkinson's disease. Trends Mol Med 11:91-96.
Farrer, M. J. 2006. Genetics of Parkinson disease: paradigm shifts and future prospects. Nat Rev Genet 7:306-318.
Ferrer, I., R. Blanco, M. Carmona, B. Puig, M. Barrachina, C. Gomez, and S. Ambrosio. 2001. Active, phosphorylation-dependent mitogen-activated protein kinase (MAPK/ERK), stress-activated protein kinase/c-Jun N-terminal kinase (SAPK/JNK), and p38 kinase expression in Parkinson's disease and Dementia with Lewy bodies. J Neural Transm (Vienna) 108:1383-1396.
Forman, M. S., V. M. Lee, and J. Q. Trojanowski. 2005. Nosology of Parkinson's disease: looking for the way out of a quagmire. Neuron 47:479-482.
Fox, S. H., R. Katzenschlager, S. Y. Lim, B. Barton, R. M. A. de Bie, K. Seppi, M. Coelho, C. Sampaio, and C. Movement Disorder Society Evidence-Based Medicine. 2018. International Parkinson and movement disorder society evidence-based medicine review: Update on treatments for the motor symptoms of Parkinson's disease. Mov Disord
Galvin, J. E., V. M. Lee, and J. Q. Trojanowski. 2001. Synucleinopathies: clinical and pathological implications. Arch Neurol 58:186-190.
Gerson, J. E., K. M. Farmer, N. Henson, D. L. Castillo-Carranza, M. Carretero Murillo, U. Sengupta, A. Barrett, and R. Kayed. 2018. Tau oligomers mediate alpha-synuclein toxicity and can be targeted by immunotherapy. Mol Neurodegener 13:13.
Grassi, D., S. Howard, M. Zhou, N. Diaz-Perez, N. T. Urban, D. Guerrero-Given, N. Kamasawa, L. A. Volpicelli-Daley, P. LoGrasso, and C. I. Lasmezas. 2018. Identification of a highly neurotoxic alpha-synuclein species inducing mitochondrial damage and mitophagy in Parkinson's disease. Proc Natl Acad Sci USA 115:E2634-E2643.
Guo, J. L., D. J. Covell, J. P. Daniels, M. Iba, A. Stieber, B. Zhang, D. M. Riddle, L. K. Kwong, Y. Xu, J. Q. Trojanowski, and V. M. Lee. 2013. Distinct alpha-synuclein strains differentially promote tau inclusions in neurons. Cell 154:103-117.
Haggerty, T., J. Credle, O. Rodriguez, J. Wills, A. W. Oaks, E. Masliah, and A. Sidhu. 2011. Hyperphosphorylated Tau in an alpha-synuclein-overexpressing transgenic model of Parkinson's disease. Eur J Neurosci 33:1598-1610.
Hansen, C., E. Angot, A. L. Bergstrom, J. A. Steiner, L. Pieri, G. Paul, T. F. Outeiro, R. Melki, P. Kallunki, K. Fog, J. Y. Li, and P. Brundin. 2011. alpha-Synuclein propagates from mouse brain to grafted dopaminergic neurons and seeds aggregation in cultured human cells. J Clin Invest 121:715-725.
Hunot, S., M. Vila, P. Teismann, R. J. Davis, E. C. Hirsch, S. Przedborski, P. Rakic, and R. A. Flavell. 2004. JNK-mediated induction of cyclooxygenase 2 is required for neurodegeneration in a mouse model of Parkinson's disease. Proc Natl Acad Sci USA 101:665-670.
Irwin, D. J., V. M. Lee, and J. Q. Trojanowski. 2013. Parkinson's disease dementia: convergence of alpha-synuclein, tau and amyloid-beta pathologies. Nat Rev Neurosci 14:626-636.
Ki, C. S., E. F. Stavrou, N. Davanos, W. Y. Lee, E. J. Chung, J. Y. Kim, and A. Athanassiadou. 2007. The Ala53Thr mutation in the alpha-synuclein gene in a Korean family with Parkinson disease. Clin Genet 71:471-473.
Kim, E. K., and E. J. Choi. 2010. Pathological roles of MAPK signaling pathways in human diseases. Biochim Biophys Acta 1802:396-405.
Klein, C., and M. G. Schlossmacher. 2007. Parkinson disease, 10 years after its genetic revolution: multiple clues to a complex disorder. Neurology 69:2093-2104.
Kordower, J. H., Y. Chu, R. A. Hauser, T. B. Freeman, and C. W. Olanow. 2008. Lewy body-like pathology in long-term embryonic nigral transplants in Parkinson's disease. Nat Med 14:504-506.
Kruger, R., W. Kuhn, T. Muller, D. Woitalla, M. Graeber, S. Kosel, H. Przuntek, J. T. Epplen, L. Schols, and O. Riess. 1998. Ala30Pro mutation in the gene encoding alpha-synuclein in Parkinson's disease. Nat Genet 18:106-108.
La Vitola, P., C. Balducci, M. Cerovic, G. Santamaria, E. Brandi, F. Grandi, L. Caldinelli, L. Colombo, M. G. Morgese, L. Trabace, L. Pollegioni, D. Albani, and G. Forloni. 2018. Alpha-synuclein oligomers impair memory through glial cell activation and via Toll-like receptor 2. Brain Behav Immun 69:591-602.
Lang, A. E., and A. M. Lozano. 1998a. Parkinson's disease. First of two parts. N Engl J Med 339:1044-1053.
Lang, A. E., and A. M. Lozano. 1998b. Parkinson's disease. Second of two parts. N Engl J Med 339:1130-1143.
Lasagna-Reeves, C. A., D. L. Castillo-Carranza, U. Sengupta, A. L. Clos, G. R. Jackson, and R. Kayed. 2011. Tau oligomers impair memory and induce synaptic and mitochondrial dysfunction in wild-type mice. Mol Neurodegener 6:39.
Li, J. Y., E. Englund, J. L. Holton, D. Soulet, P. Hagell, A. J. Lees, T. Lashley, N. P. Quinn, S. Rehncrona, A. Bjorklund, H. Widner, T. Revesz, O. Lindvall, and P. Brundin. 2008. Lewy bodies in grafted neurons in subjects with Parkinson's disease suggest host-to-graft disease propagation. Nat Med 14:501-503.
Lippa, C. F., M. L. Schmidt, V. M. Lee, and J. Q. Trojanowski. 1999. Antibodies to alpha-synuclein detect Lewy bodies in many Down's syndrome brains with Alzheimer's disease. Ann Neurol 45:353-357.
Liu, H., and M. Zhou. 2017. Antitumor effect of Quercetin on Y79 retinoblastoma cells via activation of JNK and p38 MAPK pathways. BMC Complement Altern Med 17:531.
Liu, W., A. Ruiz-Velasco, S. Wang, S. Khan, M. Zi, A. Jungmann, M. Dolores Camacho-Munoz, J. Guo, G. Du, L. Xie, D. Oceandy, A. Nicolaou, G. Galli, O. J. Muller, E. J. Cartwright, Y. Ji, and X. Wang. 2017. Metabolic stress-induced cardiomyopathy is caused by mitochondrial dysfunction due to attenuated Erk5 signaling. Nat Commun 8:494.
Luk, K. C., V. Kehm, J. Carroll, B. Zhang, P. O'Brien, J. Q. Trojanowski, and V. M. Lee. 2012. Pathological alpha-synuclein transmission initiates Parkinson-like neurodegeneration in nontransgenic mice. Science 338:949-953.
Luk, K. C., C. Song, P. O'Brien, A. Stieber, J. R. Branch, K. R. Brunden, J. Q. Trojanowski, and V. M. Lee. 2009. Exogenous alpha-synuclein fibrils seed the formation of Lewy body-like intracellular inclusions in cultured cells. Proc Natl Acad Sci USA 106:20051-20056.
Luth, E. S., I. G. Stavrovskaya, T. Bartels, B. S. Kristal, and D. J. Selkoe. 2014. Soluble, prefibrillar alpha-synuclein oligomers promote complex I-dependent, Ca2+-induced mitochondrial dysfunction. J Biol Chem 289:21490-21507.
Manczak, M., and P. H. Reddy. 2012. Abnormal interaction between the mitochondrial fission protein Drpl and hyperphosphorylated tau in Alzheimer's disease neurons: implications for mitochondrial dysfunction and neuronal damage. Hum Mol Genet 21:2538-2547.
Markopoulou, K., Z. K. Wszolek, R. F. Pfeiffer, and B. A. Chase. 1999. Reduced expression of the G209A alpha-synuclein allele in familial Parkinsonism. Ann Neurol 46:374-381.
Martin, I., V. L. Dawson, and T. M. Dawson. 2011. Recent advances in the genetics of Parkinson's disease. Annu Rev Genomics Hum Genet 12:301-325.
Martin, L., X. Latypova, C. M. Wilson, A. Magnaudeix, M. L. Perrin, C. Yardin, and F. Terro. 2013. Tau protein kinases: involvement in Alzheimer's disease. Ageing Res Rev 12:289-309.
Olanow, C. W., and W. G. Tatton. 1999. Etiology and pathogenesis of Parkinson's disease. Annu Rev Neurosci 22:123-144.
Pan, J., H. Li, B. Zhang, R. Xiong, Y. Zhang, W. Y. Kang, W. Chen, Z. B. Zhao, and S. D. Chen. 2015. Small peptide inhibitor of JNK3 protects dopaminergic neurons from MPTP induced injury via inhibiting the ASK1-JNK3 signaling pathway. PLoS One 10:e0119204.
Pankratz, N., J. B. Wilk, J. C. Latourelle, A. L. DeStefano, C. Halter, E. W. Pugh, K. F. Doheny, J. F. Gusella, W. C. Nichols, T. Foroud, and R. H. Myers. 2009. Genomewide association study for susceptibility genes contributing to familial Parkinson disease. Hum Genet 124:593-605.
Pickrell, A. M., and R. J. Youle. 2015. The roles of PINK1, parkin, and mitochondrial fidelity in Parkinson's disease. Neuron 85:257-273.
Ray, A., N. Sehgal, S. Karunakaran, G. Rangarajan, and V. Ravindranath. 2015. MPTP activates ASK1-p38 MAPK signaling pathway through TNF-dependent Trx1 oxidation in parkinsonism mouse model. Free Radic Biol Med 87:312-325.
Schapira, A. H., C. W. Olanow, J. T. Greenamyre, and E. Bezard. 2014. Slowing of neurodegeneration in Parkinson's disease and Huntington's disease: future therapeutic perspectives. Lancet 384:545-555.
Sengupta, U., M. J. Guerrero-Munoz, D. L. Castillo-Carranza, C. A. Lasagna-Reeves, J. E. Gerson, A. A. Paulucci-Holthauzen, S. Krishnamurthy, M. Farhed, G. R. Jackson, and R. Kayed. 2015. Pathological Interface between Oligomeric Alpha-Synuclein and Tau in Synucleinopathies. Biol Psychiatry
Spillantini, M. G., R. A. Crowther, R. Jakes, M. Hasegawa, and M. Goedert. 1998. alpha-Synuclein in filamentous inclusions of Lewy bodies from Parkinson's disease and dementia with lewy bodies. Proc Natl Acad Sci USA 95:6469-6473.
Spillantini, M. G., M. L. Schmidt, V. M. Lee, J. Q. Trojanowski, R. Jakes, and M. Goedert. 1997. Alpha-synuclein in Lewy bodies. Nature 388:839-840.
Spira, P. J., D. M. Sharpe, G. Halliday, J. Cavanagh, and G. A. Nicholson. 2001. Clinical and pathological features of a Parkinsonian syndrome in a family with an Ala53Thr alpha-synuclein mutation. Ann Neurol 49:313-319.
Su, C., F. Sun, R. L. Cunningham, N. Rybalchenko, and M. Singh. 2014. ERKS/KLF4 signaling as a common mediator of the neuroprotective effects of both nerve growth factor and hydrogen peroxide preconditioning. Age (Dordr) 36:9685.
Sutherland, C. 2011. What Are the bona fide GSK3 Substrates? Int J Alzheimers Dis 2011:505607.
Volpicelli-Daley, L. A., K. L. Gamble, C. E. Schultheiss, D. M. Riddle, A. B. West, and V. M. Lee. 2014a. Formation of alpha-synuclein Lewy neurite-like aggregates in axons impedes the transport of distinct endosomes. Mol Biol Cell 25:4010-4023.
Volpicelli-Daley, L. A., K. C. Luk, and V. M. Lee. 2014b. Addition of exogenous alpha-synuclein preformed fibrils to primary neuronal cultures to seed recruitment of endogenous alpha-synuclein to Lewy body and Lewy neurite-like aggregates. Nat Protoc 9:2135-2146.
Volpicelli-Daley, L. A., K. C. Luk, T. P. Patel, S. A. Tanik, D. M. Riddle, A. Stieber, D. F. Meaney, J. Q. Trojanowski, and V. M. Lee. 2011. Exogenous alpha-synuclein fibrils induce Lewy body pathology leading to synaptic dysfunction and neuron death. Neuron 72:57-71.
Wang, W., L. Shi, Y. Xie, C. Ma, W. Li, X. Su, S. Huang, R. Chen, Z. Zhu, Z. Mao, Y. Han, and M. Li. 2004. SP600125, a new JNK inhibitor, protects dopaminergic neurons in the MPTP model of Parkinson's disease. Neurosci Res 48:195-202.
Wu, R., H. Chen, J. Ma, Q. He, Q. Huang, Q. Liu, M. Li, and Z. Yuan. 2016. c-Abl-p38alpha signaling plays an important role in MPTP-induced neuronal death. Cell Death Differ 23:542-552.
Xia, X. G., T. Harding, M. Weller, A. Bieneman, J. B. Uney, and J. B. Schulz. 2001. Gene transfer of the JNK interacting protein-1 protects dopaminergic neurons in the MPTP model of Parkinson's disease. Proc Natl Acad Sci USA 98:10433-10438.
Yamasaki, T., H. Kawasaki, and H. Nishina. 2012. Diverse Roles of JNK and MKK Pathways in the Brain. J Signal Transduct 2012:459265.
Yoon, S. O., D. J. Park, J. C. Ryu, H. G. Ozer, C. Tep, Y. J. Shin, T. H. Lim, L. Pastorino, A. J. Kunwar, J. C. Walton, A. H. Nagahara, K. P. Lu, R. J. Nelson, M. H. Tuszynski, and K. Huang. 2012. JNK3 perpetuates metabolic stress induced by Abeta peptides. Neuron 75:824-837.
Yun, S. P., D. Kim, S. Kim, S. Kim, S. S. Karuppagounder, S. H. Kwon, S. Lee, T. I. Kam, S. Lee, S. Ham, J. H. Park, V. L. Dawson, T. M. Dawson, Y. Lee, and H. S. Ko. 2018. alpha-Synuclein accumulation and GBA deficiency due to L444P GBA mutation contributes to MPTP-induced parkinsonism. Mol Neurodegener 13:1.
Zarranz, J. J., J. Alegre, J. C. Gomez-Esteban, E. Lezcano, R. Ros, I. Ampuero, L. Vidal, J. Hoenicka, O. Rodriguez, B. Atares, V. Llorens, E. Gomez Tortosa, T. del Ser, D. G. Munoz, and J. G. de Yebenes. 2004. The new mutation, E46K, of alpha-synuclein causes Parkinson and Lewy body dementia. Ann Neurol 55:164-173.
Zhao, F., L. Bi, W. Wang, X. Wu, Y. Li, F. Gong, S. Lu, F. Feng, Z. Qian, C. Hu, Y. Wu, and Y. Sun. 2016. Mutations of glucocerebrosidase gene and susceptibility to Parkinson's disease: An updated meta-analysis in a European population. Neuroscience 320:239-246.
Zinchuk, V., and O. Grossenbacher-Zinchuk. 2014. Quantitative colocalization analysis of fluorescence microscopy images. Curr Protoc Cell Biol 62:Unit 4 19 11-14.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

We claim:

1. A method of generating antibodies useful for treating Parkinson's disease (PD) and other synucleinopathies, comprising (a) immunizing a non-human animal with an immunogen composition comprising an alpha-synuclein (α-syn) derived polypeptide or a polymer exhibiting the same conformational epitope as the polypeptide, and (b) isolating one or more antibodies that specifically recognize the polypeptide; wherein the α-syn derived polypeptide comprises a conformationally distinct and nonfibrillar α-syn variant with mitotoxicity.

2. The method of claim 1, wherein the α-syn derived polypeptide comprises phosphorylated Ser¹²⁹.

3. The method of claim 1, wherein the α-syn derived polypeptide is immunoreactive with anti-phospho-Ser129 antibody GTX50222, lot 821505177.

4. The method of claim 1, wherein the α-syn derived polypeptide is not immunoreactive with fibrillar Pα-synF-recognizing 81A and/or antibody MJF-R13.

5. The method of claim 1, wherein the α-syn derived polypeptide is an α-syn variant with a deletion of about 0 to 25 N-terminal amino acid residues and/or a deletion of about 0 to 25 C-terminal amino acid residues relative to a full length α-syn protein.

6. The method of claim 1, wherein the α-syn derived polypeptide is extracted from Pα-syn* inclusions present in a cell culture, brains of animal models of PD and other synucleinopathies, or brains of patients with PD and other synucleinopathies.

7. The method of claim 1, wherein the immunogen composition further comprises an adjuvant.

8. The method of claim 1, wherein the antibody is isolated by phage display.

9. The method of claim 1, further comprising examining the isolated antibodies for a therapeutic activity.

10. The method of claim 9, wherein the therapeutic activity is inhibition of a toxic activity in a cellular or organism model of synucleinopathy.

11. The method of claim 14, wherein the therapeutic activity is a reduction in the generation and propagation of pathogenic phosphorylated α-syn.

12. The method of claim 1, wherein the polypeptide is derived from a human α-syn.

13. The method of claim 12, wherein the human α-syn comprises at least a 50% sequence identity to SEQ ID NO:1, variants or fragments thereof.

14. The method of claim 13, wherein the human α-syn comprises SEQ ID NO:1, varaints or fragments thereof.

15. The method of claim 1, wherein the mitotoxicity is inducing mitochondrial dysfunction and structural damage resulting in mitophagy

16. A method for identifying potential therapeutic agents for treating PD and other synucleinopathies, comprising (a) contacting with or administering to a cell or animal model of PD and other synucleinopathies a plurality of candidate agents, (b) detecting in a specific candidate agent-treated model a disruption or decreased formation of an alpha-synuclein (α-syn) derived polypeptide relative to untreated control model, thereby identifying the specific candidate agent as a potential therapeutic agent for treating PD and other synucleinopathies; wherein the α-syn derived polypeptide comprises a conformationally distinct and nonfibrillar α-syn variant with mitotoxicity.

17. The method of claim 16, wherein the α-syn derived polypeptide comprises phosphorylated Ser¹²⁹.

18. The method of claim 16, wherein the α-syn derived polypeptide is an α-syn variant with a deletion of about 0 to 25 N-terminal amino acid residues and/or a deletion of about 0 to 25 C-terminal amino acid residues relative to a full length α-syn protein.

19. The method of claim 16, wherein the mitotoxicity is inducing mitochondrial dysfunction and structural damage resulting in mitophagy.

20. The method of claim 16, wherein the potential therapeutic agent inhibits α-syn derived polypeptide mediated formation of phosphorylated acetyl-CoA carboxylase (ACC) aggregates.

21. The method of claim 16, wherein the potential therapeutic agent inhibits α-syn derived polypeptide mediated phosphorylation of glycogen synthase kinase 3 beta (GSK3β) and mitogen-activated protein kinases (MAPKs) comprising mitogen-activated protein kinase kinase 4 (MKK4), c-Jun N-terminal kinase (JNK), p38 or extracellular signal-regulated kinase 5 (ERK5).

22. The method of claim 16, wherein the potential therapeutic agent inhibits α-syn derived polypeptide mediated formation of phosphorylated tau aggregates.

23. The method of claim 16, wherein the potential therapeutic agent inhibits α-syn derived polypeptide mediated synaptic toxicity and loss of dendritic spines of neurons.

24. The method of claim 16, wherein the α-syn derived polypeptide is immunoreactive with anti-phospho-Ser129 antibody GTX50222, lot 821505177.

25. The method of claim 16, wherein the α-syn derived polypeptide is not immunoreactive with fibrillar Pα-synF-recognizing 81A and/or antibody MJF-R13.

26. The method of claim 16, further comprising examining the potential therapeutic agent for a therapeutic activity.

27. The method of claim 26, wherein the therapeutic activity is inhibition of a toxic activity in a cellular or organism model of synucleinopathy.

28. The method of claim 26, wherein the therapeutic activity is a reduction in the generation and propagation of pathogenic phosphorylated α-syn.

29. The method of claim 16, wherein the polypeptide is derived from a human α-syn.

30. The method of claim 29, wherein the human α-syn comprises at least a 50% sequence identity to SEQ ID NO:1, variants or fragments thereof.

31. The method of claim 30, wherein the human α-syn comprises SEQ ID NO:1, variants or fragments thereof.

32. A method for identifying potential therapeutic agents for treating PD and other synucleinopathies, comprising (a) contacting with or administering to a cell or animal model of PD and other synucleinopathies or a α-syn derived polypeptide extracted from Pα-syn* inclusions present in a cell culture, brains of animal models, or brains of patients with PD and other synucleinopathies. a plurality of candidate agents, (b) detecting binding of a candidate agent to a alpha-synuclein (α-syn) derived polypeptide specific to the candidate agent-treated relative to untreated control model, thereby identifying the specific candidate agent as a potential therapeutic agent for treating PD and other synucleinopathies; wherein the α-syn derived polypeptide comprises a conformationally distinct and nonfibrillar α-syn variant with mitotoxicity.

33. The method of claim 32, wherein the α-syn derived polypeptide comprises phosphorylated Ser¹²⁹.

34. The method of claim 32, wherein the α-syn derived polypeptide is an α-syn variant with a deletion of about 0 to 25 N-terminal amino acid residues and/or a deletion of about 0 to 25 C-terminal amino acid residues relative to a full length α-syn protein.

35. The method of claim 32, wherein the mitotoxicity is inducing mitochondrial dysfunction and structural damage resulting in mitophagy.

36. The method of claim 32, wherein the potential therapeutic agent inhibits α-syn derived polypeptide mediated formation of phosphorylated acetyl-CoA carboxylase (ACC) aggregates.

37. The method of claim 32, wherein the potential therapeutic agent inhibits α-syn derived polypeptide mediated phosphorylation of glycogen synthase kinase 3 beta (GSK3β) and mitogen-activated protein kinases (MAPKs) such as mitogen-activated protein kinase kinase 4 (MKK4), c-Jun N-terminal kinase (JNK), p38 and extracellular signal-regulated kinase 5 (ERK5).

38. The method of claim 32, wherein the potential therapeutic agent inhibits α-syn derived polypeptide mediated formation of phosphorylated tau aggregates.

39. The method of claim 32, wherein the potential therapeutic agent inhibits α-syn derived polypeptide mediated synaptic toxicity and loss of dendritic spines of neurons.

40. The method of claim 32, wherein the α-syn derived polypeptide is immunoreactive with anti-phospho-Ser129 antibody GTX50222, lot 821505177.

41. The method of claim 32, wherein the α-syn derived polypeptide is not immunoreactive with fibrillar Pα-synF-recognizing 81A and/or antibody MJF-R13.

42. The method of claim 32, further comprising examining the potential therapeutic agent for a therapeutic activity.

43. The method of claim 42, wherein the therapeutic activity is inhibition of a toxic activity in a cellular or organism model of synucleinopathy.

44. The method of claim 42, wherein the therapeutic activity is a reduction in the generation and propagation of pathogenic phosphorylated α-syn.

45. The method of claim 32, wherein the polypeptide is derived from a human α-syn.

46. The method of claim 32, wherein the human α-syn comprises at least a 50% sequence identity to SEQ ID NO:1, variants or fragments thereof.

47. The method of claim 34, wherein the human α-syn comprises SEQ ID NO:1, variants or fragments thereof.

48. A method of diagnosing or monitoring disease progression in patients affected by PD and other synucleinopathies, comprising detecting the presence and/or quantifying the amount of a conformationally distinct and nonfibrillar α-syn variant with mitotoxicity.

49. The method of claim 48, wherein the α-syn derived polypeptide comprises phosphorylated Ser¹²⁹.

50. The method of claim 48, wherein the α-syn variant comprises a deletion of about 0 to 25 N-terminal amino acid residues and/or a deletion of about 0 to 25 C-terminal amino acid residues relative to a full length α-syn protein.

51. The method of claim 48, wherein the mitotoxicity is inducing mitochondrial dysfunction and structural damage resulting in mitophagy.

52. The method of claim 48, wherein the method detects α-syn variant mediated formation of phosphorylated acetyl-CoA carboxylase (ACC) aggregates.

53. The method of claim 48, wherein the method detects α-syn variant mediated phosphorylation of glycogen synthase kinase 3 beta (GSK3β) and mitogen-activated protein kinases (MAPKs) such as mitogen-activated protein kinase kinase 4 (MKK4), c-Jun N-terminal kinase (JNK), p38 and extracellular signal-regulated kinase 5 (ERK5).

54. The method of claim 48, wherein the method detects α-syn variant mediated formation of phosphorylated tau aggregates.

55. The method of claim 48, wherein the method detects α-syn variant mediated synaptic toxicity and loss of dendritic spines of neurons.

56. The method of claim 48, wherein the diagnosis or disease monitoring is performed with a tissue or body fluid sample obtained from subjects affected by PD and other synucleinopathies.

57. An engineered cell or transgenic non-human animal comprising a transgene encoding an alpha-synuclein (α-syn) derived polypeptide, wherein the α-syn derived polypeptide consists of a deletion of about 0 to 25 N-terminal amino acid residues and a deletion of about 0 to 25 C-terminal amino acid residues of a full length α-syn protein.

58. The engineered cell of claim 57, which is a neuronal cell.

59. The transgenic non-human animal of claim 57, which is a rodent.

60. An engineered cell or transgenic non-human animal comprising a transgene encoding an alpha-synuclein (α-syn) derived polypeptide, wherein the α-syn derived polypeptide harbours a mutation in one or several amino acid residues of a full length or truncated α-syn protein rendering the α-syn variant prone to adopt a distinct and nonfibrillar α-syn conformation with mitotoxicity.

61. The engineered cell of claim 60, which is a neuronal cell.

62. The transgenic non-human animal of claim 60, which is a rodent.

63. A method of generating small molecules useful for treating Parkinson's disease (PD) and other synucleinopathies, comprising (a) performing structure-based drug design directed towards the conformational epitope of an immunogen composition comprising an alpha-synuclein (α-syn) derived polypeptide or a polymer exhibiting the same conformational epitope as the polypeptide, and (b) selecting a small molecule specifically recognizing the conformational epitope of an α-syn derived polypeptide; wherein the α-syn derived polypeptide comprises a conformationally distinct and nonfibrillar α-syn variant with mitotoxicity.

64. The method of claim 63, wherein the α-syn derived polypeptide comprises phosphorylated Ser¹²⁹.

65. The method of claim 63, wherein the α-syn derived polypeptide is immunoreactive with anti-phospho-Ser129 antibody GTX50222, lot 821505177.

66. The method of claim 63, wherein the α-syn derived polypeptide is not immunoreactive with fibrillar Pα-synF-recognizing 81A and/or antibody MJF-R13.

67. The method of claim 64, wherein the α-syn derived polypeptide is an α-syn variant with a deletion of about 0 to 25 N-terminal amino acid residues and/or a deletion of about 0 to 25 C-terminal amino acid residues relative to a full length α-syn protein.

68. The method of claim 63, wherein the mitotoxicity is inducing mitochondrial dysfunction and structural damage resulting in mitophagy.

69. The method of claim 63, wherein the small molecule inhibits α-syn derived polypeptide mediated formation of phosphorylated acetyl-CoA carboxylase (ACC) aggregates.

70. The method of claim 63, wherein the small molecule inhibits α-syn derived polypeptide mediated phosphorylation of glycogen synthase kinase 3 beta (GSK3β) and mitogen-activated protein kinases (MAPKs) such as mitogen-activated protein kinase kinase 4 (MKK4), c-Jun N-terminal kinase (JNK), p38 and extracellular signal-regulated kinase 5 (ERK5).

71. The method of claim 63, wherein the small molecule inhibits α-syn derived polypeptide mediated formation of phosphorylated tau aggregates.

72. The method of claim 63, wherein the small molecule inhibits α-syn derived polypeptide mediated synaptic toxicity and loss of dendritic spines of neurons.

73. The method of claim 63, wherein the α-syn derived polypeptide is extracted from Pα-syn* inclusions present in a cell culture, brains of animal models of PD and other synucleinopathies, or brains of patients with PD and other synucleinopathies.

74. The method of claim 63, further comprising examining the selected small molecule for a therapeutic activity.

75. The method of claim 74, wherein the therapeutic activity is inhibition of a toxic activity in a cellular model of synucleinopathy or a reduction in the generation and propagation of pathogenic phosphorylated α-syn.

76. The method of claim 63, wherein the polypeptide is derived from a human α-syn.

77. The method of claim 76, wherein the human α-syn comprises at least a 50% sequence identity to SEQ ID NO:1, variants or fragments thereof.

78. The method of claim 77, wherein the human α-syn consists of SEQ ID NO:1.


SEQUENCE LISTING (3 sequences)

SEQ ID NO: 1 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 2 - protein, 11 aa, Artificial Sequence
  Synthetic construct palpha-synF
AYEMPSEEGYQ

SEQ ID NO: 3 - protein, 13 aa, Artificial Sequence
  Synthetic construct palpha-syn*
EAYEMPSEEGYQD